(19) United States
(12) Patent Application Publication
(10) Pub. No.: US 2016/0324954 A1
(43) Pub. Date: Nov. 10, 2016

(54) IMMUNOGENIC COMPOSITIONS FOR INHIBITING HEPATITIS D VIRUS

(71) Applicant: CHRONTECH PHARMA AB, Huddinge (SE)

(72) Inventors: Matti Sällberg, Stockholm (SE); Lars Frelin, Älvsjö (SE); Gustaf Ahlen, Järfälla (SE)

(21) Appl. No.: 14/891,150

(22) PCT Filed: May 13, 2014
(86) PCT No.: PCT/US2014/037918

§ 371 Date: Nov. 13, 2015

Related U.S. Application Data
(60) Provisional application No. 61/823,760, filed on May 15, 2013.

(51) Int. Cl. A61K 39/29; C12N 7/00

(52) U.S. Cl. CPC A61K 39/29; C12N 7/00; A61K 39/292; A61K 2039/545

(57) ABSTRACT

Disclosed herein are isolated nucleic acids, compositions of isolated nucleic acids, and compositions of polypeptides that are useful for the generation, enhancement, or improvement of an immune response to a target antigen. Some embodiments of the compositions include nucleic acids encoding hepatitis B core antigen (HBcAg) protein in combination with one or more self-cleavage 2A polypeptides and a second antigenic polypeptide. In certain embodiments, the HBcAg is as a stork or heron hepatitis antigen. In certain embodiments the self-cleavage polypeptide is P2A. In certain embodiments the second antigenic polypeptide of interest is NS5A, HDAg, or birch allergen.

### REFERENCE TO SEQUENCE LISTING, TABLE, OR COMPUTER PROGRAM LISTING

The present application is being filed along with a Sequence Listing in electronic format. The Sequence Listing is provided as a file entitled TRIPEP134WO_SEQUENCE_LISTING.TXT. The information in the electronic format of the Sequence Listing is incorporated herein by reference in its entirety.

### FIELD OF THE INVENTION

The disclosure herein relates to the field of immunology and, more specifically, the development of isolated nucleic acids, nucleic acid immunogens, nucleic acid immunogenic compositions and DNA vaccines, which can be used to inhibit or treat hepatitis D virus infections.

### BACKGROUND OF THE INVENTION

Traditionally, vaccines have been based on live attenuated or inactivated pathogens. These strategies are inefficient, however, largely because of the antigenic variability of pathogens (e.g., viruses). Several peptide vaccines that comprise antigenic peptides or peptide fragments of pathogens have been developed. Conserved peptide fragments are less likely to exhibit antigenic variability and can overcome some of the problems associated with traditional peptides. Accordingly, subunit vaccines have been developed, which target conserved regions of pathogens. Synthetic peptide vaccines tend to be poorly immunogenic, however. The poor immunogenicity of synthetic peptide vaccines may be attributed to the fact that although these types of vaccines induce humoral antibody responses, they are less likely to induce cell-mediated responses.

Several investigators have sought to improve the antigenicity of synthetic peptide vaccines. For example, Klein et al. describe the engineering of chimeric proteins that comprise an immunogenic region of a protein from a first antigen linked to an immunogenic region from a second pathogen (See, U.S. Pat. Nos. 6,033,668; 6,017,539; 5,998,169; and 5,968,776). Others have sought to create chimeric proteins that couple B-cell epitopes to universal T-cell epitopes in order to improve the immune response. (See, e.g., U.S. Pat. No. 5,114,713). Russell-Jones et al. (U.S. Pat. No. 5,928,644) also disclose T-cell epitopes derived from the TraT protein of Escherichia coli, which are used to produce hybrid molecules so as to generate an immune response to parasites, soluble factors (e.g., LSH) and viruses. Further, Ruslan (U.S. Application Publication No. 2003/0232055) discloses the manufacture of vaccines based on PAMPs and immunogenic antigens.

Hepatitis is a disease resulting in swelling and inflammation of the liver. This disorder is commonly caused by viruses, five types of which are currently known (Hepatitis A, B, C, D and E). Hepatitis D virus (HDV), also referred to as Hepatitis delta virus, is a small, spherical single-stranded circular RNA virus. The entire virus was cloned and sequenced in 1986, and given the genus of Deltavirus. HDV is structurally unrelated to the other hepatitis viruses. Since HDV is an incomplete virus, it can only replicate in the presence of Hepatitis B (HBV) virus, which provides structural components for HDV. In particular, HDV has an outer coat that contains large, medium and small hepatitis B surface antigens, and host lipids surrounding an inner nucleocapsid, which contains about 200 molecules of hepatitis D antigen (HDAg) for each genome. The circular genome of HDV is unique to animal viruses because of its high GC content.

HDV produces a single protein, namely hepatitis D antigen (HDAg). HDAg exists in two isoforms: a 27 kDa large-HDAg (HDAg-L), and a 24 kDa small-HDAg (HDAg-S). The two sequences differ in that the C-terminus of the HDAg-L contains an additional 19 amino acids not found in HDAg-S, which are essential to virus assembly. Both isoforms are produced from the same open reading frame (ORF), which contains a UAG stop codon at codon 196, which normally produces only the HDAg-S. However, editing by the cellular enzyme adenosine deaminase-1 changes the stop codon to UCG, allowing HDAg-L to be produced. HDAg-S is produced in the early stages of infection, enters the nucleus and supports viral replication. In contrast, HDAg-L is produced during the later stages of infection, acts as an inhibitor of viral replication, and is required for assembly of viral particles. Both isoforms bind RNA, with a specificity for the rod-like folding of the HDV genome and antigenome (Chao et al., J. Virol. 65:4057-4062, 1991; Lee et al., J. Virol., 67:2221-2227, 1993). HDAg contains a coiled-coil dimerization domain, nuclear localization signal, RNA-binding domain, and a putative assembly domain. Various epitopes of HDAg were determined to be exposed by PEPSCAN, immunoprecipitation analysis and ELISA, including those within amino acids 12-60, 58-78, 82-102, 123-143, 156-184, 167-184 and 197-211 (Bichko et al., (1996) J. Virol. 70:5807-5811). Epitope mapping of HDAg in patients with chronic Hepatitis D infection exhibited the following potential cytotoxic T-ligand epitopes: amino acids 43 to 51, 50 to 58 and 114 to 122 (Wang et al., J. Virol., 81:4438-4444, 2007).

HDV is transmitted through percutaneous or mucosal contact with infected blood. HDV can be acquired by either simultaneous infection with HBV (coinfection), or by superinfection, in, which HDV is superimposed on chronic HBV infection or carrier state. Both types of infection result in more deleterious effects than infection solely with HBV, including enhanced possibility of liver failure and more rapid onset of cirrhosis and potentially liver cancer. The combination of HBV and HDV results in the highest mortality rate of all hepatitis infections at about 20%. There is no current vaccine for HDV, but it can be prevented in individuals who are not already infected with HBV by HBV vaccination.

DNA vaccines can be used as a model to study the endogenous immunogenicity of antigens. However, phase I/II clinical trials reveal that it is difficult to prime robust immune responses in humans with direct intramuscular injections of DNA vaccines. Different modes of DNA delivery have now become available, including transdermal delivery of DNA coated to gold beads using a gene gun or in vivo electroporation technologies.

With respect to DNA vaccines, studies have been rather disappointing (Kutzler M A, Weiner D B. (2008) DNA vaccines: ready for prime time? Nat Rev Genet. 9(10):776-88). However, new technologies can improve the immunogenicity of plasmid DNA, for example in vivo electroporation (EP), (see e.g., U.S. patent application Ser. No. 13/514,269 and U.S. Pat. App. No. PCT/IB2012/001321, the disclosures of which are hereby expressly incorporated by reference in their entireties). With EP, electrical pulses are administered, which cause permeabilization of cellular membranes that increase DNA uptake and vaccine expression, and, which also generates a local inflammatory response (Ahlen G, Soderholm J, Tjelle T E, Kjeken R, Frelin L, Hoglund U, et al. (2007) In vivo Electroporation Enhances the Immunogenicity of Hepatitis C Virus Nonstructural3/4A DNA by Increased Local DNA Uptake, Protein Expression, Inflammation, and Infiltration of CD3+ cells. J Immunol. 179(7):4741-53). This technique has been used in cancer patients (Rice J, Ottensmeier C H, Stevenson F K. DNA vaccines: precision tools for activating effective immunity against cancer. Nat Rev Cancer. 2008 February; 8(2):108-20), and has been found to raise T cell responses to HCV in chimpanzees (Folgori A, Capone S, Ruggeri L, Meola A, Sporeno E, Ercole B B, et al. A Tcell HCV vaccine eliciting effective immunity against heterologous virus challenge in chimpanzees. Nat Med. 2006 February; 12(2):190-7).

Substantial progress has been made in the field of DNA vaccination, however, the challenges of diseases such as HDV are persistent, and the need for approaches that enhance the immune response of a subject after vaccination, in particular DNA vaccination, is ongoing.

### SUMMARY OF THE INVENTION

Several embodiments described herein concern isolated nucleic acids, expression constructs, DNA immunogenic compositions, DNA vaccines or nucleic acid immunogens, preferably, which are codon-optimized for expression in humans, and that encode a peptide that comprises, consists of, or consists essentially of a first antigenic sequence, which is an HBcAg sequence, (e.g., a polypeptide of or encoded by SEQ ID NOs:1-10) preferably full-length, (e.g., an avian HBcAg, such as stork, heron, or duck), wherein said HBcAg sequence comprises a self-cleavage site or domain that exists within said HBcAg sequence or joined to said HBcAg sequence at the N or C terminus Exemplary self-cleavage domains, also referred to as ‘self-cleavage 2A peptide sequences’ that can be used in these embodiments include porcine teschovirus-1 2A (P2A, a polypeptide of or encoded by SEQ ID NOs:11-12), foot-and-mouth disease virus (FMDV) 2A (F2A, a polypeptide of or encoded by SEQ ID NOs:13-14), equine rhinitis A virus (ERAV) 2A (E2A, a polypeptide of or encoded by SEQ ID NOs:13-14), and Thosea asigna virus 2A (T2A, a polypeptide of or encoded by SEQ ID NOs:13-14), wherein each self-cleavage sequence can be modified to include a GSG (glycine-serine-glycine, a polypeptide of or encoded by SEQ ID NOs:19-20) motif at the N-terminus, which is contemplated to improve cleavage efficiency. See, e.g., SEQ ID NOs:21-22. The above-mentioned self-cleavage sequences and GSG enhancer may be joined to said HBcAg polypeptides, for example as seen in the polypeptides of or encoded by SEQ ID NOs:23-30). An additional nucleic acid encoding a second antigenic sequence, preferably also codon optimized for expression in humans, (e.g., a nucleic acid encoding a peptide comprising an HDV polypeptide (e.g., HDAg-L or HDAg-S, a nucleotide of or encoding SEQ ID NOs:33-40) or an antigenic fragment thereof, such as a fragment that is 30, 40, 50, 60, 70, 80, 90, or 100 amino acids in length or that is, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the full length sequence) can be joined to said first antigenic sequence (i.e., the HBcAg sequence having said self-cleavage sites or domains, described above, such as a nucleotide of or encoding SEQ ID NOs:23-30) so as to generate a fusion protein having said self-cleavage sites, such as a protein of or encoded by SEQ ID NOs:105-120, or alternate proteins of or encoded by combinations of SEQ ID NOs:1-10, 11-22, 23-30, ans 33-40. Preferably, the self-cleavage site exists or is introduced between said HBcAg sequence and said second antigenic sequence (e.g., the peptide comprising an HDV polypeptide (e.g., HDAg-L or HDAg-S) or an antigenic fragment thereof, such as a fragment that is 30, 40, 50, 60, 70, 80, 90, or 100 amino acids in length or that is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the full length sequence). These embodiments are useful for the generation, inducement, enhancement, or improvement of an immune response (e.g., a T cell-specific immune response and/or an antibody-specific immune response) to the target antigen encoded by the first and/or second antigenic sequence (e.g., a peptide comprising at least an antigenic fragment of HDV (e.g., HDAg-L or HDAg-S), as described above). These compositions are particularly useful for the treatment or inhibition of HDV infection. Preferably, one or more or all of these sequences are codon optimized for expression in humans. Methods of using the foregoing compositions to generate an immune response (e.g., a T cell and/or antibody specific immune response) or to treat an HDV infection in a subject, preferably a human and, optionally a chronically infected human, are contemplated embodiments. Optionally, a subject can be identified as one in need of an immune response to HDV prior to administration of the composition and/or said subject can be evaluated for the immune response or viral clearance after administration of said compositions and such identification and/or evaluation can be accomplished using readily available diagnostics and/or clinical approaches.

In this disclosure, it is revealed that HBcAg, in particular non-human HBcAgs, such as those derived from an avian hepatitis virus, preferably, the virus that infects stork, heron, or duck, demonstrate an improved ability to elicit an immune response to a co-administered antigen when the HBcAg (such as SEQ ID NOs:1-4) is modified to include one or more self-cleavage sites or domains (e.g., a 2A sequence such as a P2A sequence SEQ ID NOs:11-12, 21-22 or other 2A sequence such as SEQ ID NOs:13-18), which can be introduced internally in the HBcAg sequence or at the N or C termini or both (such as in SEQ ID NOs:23-30 as examples). It is contemplated that a nucleic acid immunogen, preferably also codon optimized for expression in humans, (e.g., a DNA immunogenic composition or vaccine) comprising an open reading frame encoding a target antigen polypeptide will be a stronger immunogen if the target antigenic polypeptide is encoded in a common open reading frame with a sequence encoding said HBcAg polypeptide and one or more self-cleavage sites or domains.

Preferably, the immunogen or construct comprises a nucleic acid that encodes an avian HBcAg sequence (e.g., a stork (SEQ ID NOs:1-2) or heron (SEQ ID NOs: 3-4) HBcAg sequence) comprising a self-cleavage site or domain (e.g., P2A, T2A, E2A, or F2A, with or without an N-terminal GSG motif, SEQ ID NOs:11-22) that separates the target antigenic polypeptide (e.g., a peptide comprising HDV or a fragment thereof, such as 30, 40, 50, 60, 70, 80, 90, or 100 amino acids in length, SEQ ID NOs:33-40, for example) from the HBcAg polypeptide. Preferably, this sequence is codon optimized for expression in humans. For example, the target antigen is joined to the avian HBcAg such that the self-cleavage site exists between said HBcAg and said target antigen (see, e.g., SEQ ID NOs:58-59, 62-63, 66-67, 70-71, or other HDAg bound to, for example, SEQ ID NOs:23-26). Alternatively, the self-cleavage sites or domains (e.g., P2A, T2A, E2A, or F2A, with or without an N-terminal GSG motif) are incorporated within said HBcAg, such that said HBcAg having the self-cleavage sites or domains generate fragments of HBcAg upon self-cleavage (see, e.g., SEQ ID NOs:27-30). Preferably, this peptide is encoded by a nucleic acid sequence that is codon optimized for expression in humans and this nucleic acid can be used as an immunogen to inhibit HDV infection or proliferation. In another embodiment, the self-cleavage site or domain (e.g., e.g., P2A, T2A, E2A, or F2A, with or without an N-terminal GSG motif) exists between said avian HBcAg (e.g., stork or heron HBcAg) and said target antigenic polypeptide (e.g., a peptide comprising a HDV polypeptide (e.g., HDAg-L or HDAg-S) or a fragment thereof, such as 30, 40, 50, 60, 70, 80, 90, or 100 amino acids in length), such that the self-cleavage site separates the target antigenic polypeptide from the HBcAg polypeptide and self-cleavage sites or domains (e.g., P2A, T2A, E2A, or F2A, with or without an N-terminal GSG motif) exist within the HBcAg polypeptide, such that said HBcAg having the self-cleavage sites or domains generate fragments of HBcAg upon self-cleavage (see, e.g., SEQ ID NOs:27-30, 60-61, 64-65, 68-69, 72-73). Preferably, one or more or all of these sequences are codon optimized for expression in humans. Methods of using the foregoing compositions to generate an immune response (e.g., a T cell and/or antibody specific immune response) or to treat an HDV infection in a subject, preferably a human and, optionally a chronically infected human, are contemplated embodiments. Optionally, a subject can be identified as one in need of an immune response to HDV prior to administration of the composition and/or said subject can be evaluated for the immune response or viral clearance after administration of said compositions and such identification and/or evaluation can be accomplished using readily available diagnostics and/or clinical approaches.

Ideally, an HBcAg derived from a hepatitis virus that does not infect a human (a “non-human HBcAg”) or a nucleic acid encoding said non-human HBcAg (e.g., an HBcAg derived from an avian hepatitis virus, such as the hepatitis virus that infects stork or heron (e.g., SEQ ID NOs:1-4) is encoded in an open reading frame with a target antigen (e.g., a nucleic acid encoding a peptide comprising a HDV polypeptide (e.g., HDAg-L or HDAg-S) or a fragment thereof, such as 30, 40, 50, 60, 70, 80, 90, or 100 amino acids in length, such as SEQ ID NOs:33-40). Preferably, this peptide is encoded by a nucleic acid sequence that is codon optimized for expression in humans and this nucleic acid can be used as an immunogen to inhibit HDV infection or proliferation. HBV now afflicts almost a third of the world's population. Accordingly, a significant amount of the population has antibodies that react to an HBcAg derived from a hepatitis virus that infects humans. By utilizing avian HBcAg sequences, the compositions described herein can be made suitable for introduction into subjects that are already infected with HBV or subjects that have already generated antibodies to HBV (e.g., a subject that had been previously inoculated with an HBV vaccine). Preferably, the nucleic acid sequences encoding the aforementioned fusion proteins (e.g., a nucleic acid encoding a fusion protein comprising an avian HBcAg joined to a self-cleavage site, such as P2A, which is also joined to a target antigen, such as a peptide comprising HDV (e.g., HDAg-L or HDAg-S) or a fragment thereof, such as 30, 40, 50, 60, 70, 80, 90, or 100 amino acids in length) are codon-optimized for expression in the subject (e.g., codon-optimized for expression in the particular animal or human (e.g., SEQ ID NOs:1, 3). Methods of using the foregoing compositions to generate an immune response (e.g., a T cell and/or antibody specific immune response) or to treat a HDV infection in a subject, preferably a human and, optionally a chronically infected human, are contemplated embodiments. Optionally, a subject can be identified as one in need of an immune response to HDV prior to administration of the composition and/or said subject can be evaluated for the immune response or viral clearance after administration of said compositions and such identification and/or evaluation can be accomplished using readily available diagnostics and/or clinical approaches.

Preferably, at least one ‘self-cleavage’ 2A polypeptide is encoded in an open reading frame with the target antigen and the HBcAg (e.g., a nucleotide of or encoding SEQ ID NOs:1-10), e.g., a non-human HBcAg (e.g., SEQ ID NOs:1-4), such as an HBcAg from an avian hepatitis virus, including a hepatitis virus that infects stork, heron, or duck, as seen in SEQ ID NOs:1-4. Again, exemplary 2A peptides include porcine teschovirus-1 2A (P2A), equine rhinitis A virus (ERAV) 2A (E2A), foot-and-mouth disease virus (FMDV) 2A (F2A), and Thosea asigna virus 2A (T2A), see, e.g., SEQ ID NOs: 11-18, although other sequences are contemplated. In some embodiments, the 2A self-cleavage polypeptide, such as P2A, T2A, E2A, or F2A, for example, is supplemented with a Glycine-Serine Glycine sequence (GSG sequence, SEQ ID NOs:19-20, for example) at its N-terminus to increase self-cleavage efficiency, as seen in SEQ ID NOs:21-22). Preferably, these peptides are encoded by a nucleic acid sequence that is codon optimized for expression in humans and this nucleic acid can be used as an immunogen to inhibit HDV infection or proliferation.

Accordingly, several aspects of the invention described herein concern compositions that comprise, consist essentially of, or that consist of nucleic acids that encode an HBcAg of an avian hepatitis virus (e.g., a hepatitis virus that infects stork or heron (e.g., SEQ ID NOs:1-4), which may be codon-optimized for expression in humans, and, which can be joined (e.g., in Cis) to a nucleic acid (preferably codon-optimized for expression in an animal or human) that encodes a target antigen (e.g., a HDV polypeptide such as, HDAg-L or HDAg-S or a fragment thereof, such as 30, 40, 50, 60, 70, 80, 90, or 100 amino acids in length SEQ ID NOs:33-40), and which further comprise nucleic acids that encode one or more self-cleavage sequences or domains (e.g., P2A, T2A, E2A, or F2A, SEQ ID NOs:11-18) that exist between the nucleic acid encoding the target antigen and the nucleic acid encoding the HBcAg, and, which may optionally, exist within the nucleic acid sequence encoding the HBcAg polypeptide such that the translated HBcAg is self-cleaved into polypeptide fragments, such as SEQ ID NOs:21-30, and/or optionally may include one or more nucleic acid sequences encoding self-cleavage 2A polypeptides (e.g., P2A, T2A, E2A, or F2A) that exist within the sequence encoding the target antigen such that the translated target antigen is self-cleaved into polypeptide fragments, resulting in, for example a nucleic acid sequence of or encoding polypeptide sequence, such as SEQ ID NOs:27-30. Preferably, one or more or all of these sequences are codon optimized for expression in humans. Methods of using the foregoing compositions to generate an immune response (e.g., a T cell and/or antibody specific immune response) or to treat HDV infection in a subject, preferably a human and, optionally a chronically infected human, are contemplated embodiments. Optionally, a subject can be identified as one in need of an immune response to HDV prior to administration of the composition and/or said subject can be evaluated for the immune response or viral clearance after administration of said compositions and such identification and/or evaluation can be accomplished using readily available diagnostics and/or clinical approaches.

Compositions or mixtures that further comprise, consist essentially of, or that consist of one or more of nucleic acids (e.g., in Trans) that encode polypeptide adjuvants, such as nucleic acids encoding IL-12, IL-15, or IL-21 (SEQ ID NOs:74, 76, 78), which may optionally be codon optimized for expression in humans, or that consist of polypeptide adjuvants IL-12, IL-15, or IL-21 (see, for example SEQ ID NOs:75, 77, 79) or that consist of small molecule adjuvants such as ribavirinn or CpG nucleic acids are also embodiments. Preferably, these nucleic acids are codon optimized for expression in humans and these nucleic acids can be used as an immunogen to inhibit HDV infection or proliferation. Methods of using the aforementioned compositions to improve, enhance, or generate an immune response in a subject or to treat diseases such as HDV, especially in chronically infected individuals, are also contemplated.

Accordingly, several embodiments disclosed herein include an isolated nucleic acid comprising a sequence that encodes a hepatitis B virus HBcAg polypeptide or an antigenic portion thereof (for example, sequences of or encoding SEQ ID NOs:1-10), which comprises one or more self-cleavage sequences (for example, sequences of or encoding SEQ ID NOs:11-18), that exist within said HBcAg polypeptide or antigenic portion thereof and/or said one or more self-cleavage sequences are joined to said HBcAg polypeptide or antigenic portion thereof (for example, sequences of or encoding SEQ ID NOs:23-30). Preferably, these nucleic acids are codon optimized for expression in humans and these nucleic acids can be used as an immunogen to inhibit HDV infection or proliferation.

In some aspects, the nucleic acid comprises a sequence having a contiguous open reading frame such that at least a portion of said contiguous open reading frame encodes the HBcAg polypeptide or antigenic portion thereof and at least a portion of said contiguous open reading frame encodes said one or more self-cleavage sequence(s), as seen in example SEQ ID NOs:58, 60, 62, 64, 66, 68, 70, 72) although additional examples are contemplated.

In some aspects, the self-cleavage sequence(s) are selected from the group consisting of P2A, E2A, F2A, and T2A (see, for example SEQ ID NOs:11-18). In some aspects, the self-cleavage sequence may exist within said HBcAg polypeptide or antigenic portion thereof, and in some aspects the self-cleavage sequence is joined to said HBcAg polypeptide or antigenic portion thereof at the N or C terminus Preferably, these nucleic acids are codon optimized for expression in humans and these nucleic acids can be used as an immunogen to inhibit HDV infection or proliferation.

In some aspects, the HBcAg polypeptide or antigenic portion thereof comprises a full length HBcAg polypeptide, a 150 amino acid fragment of an HBcAg polypeptide, a 100 amino acid fragment of an HBcAg polypeptide, or a 50 amino acid fragment of an HBcAg polypeptide. In some aspects, HBcAg polypeptide or antigenic portion thereof is an avian HBcAg polypeptide or antigenic portion thereof. In some aspects, HBcAg polypeptide or antigenic portion thereof is a stork, duck, or heron HBcAg polypeptide or antigenic portion thereof. In some aspects, the isolated nucleic acid encoding said HBcAg polypeptide or antigenic portion thereof and/or said self-cleavage site is codon optimized for expression in humans. In some embodiments, the nucleic acid sequence encodes greater than or equal to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the HBcAg polypeptide. Optionally, these sequences can be codon optimized for expression in humans. In some embodiments, the nucleic acid sequence encodes greater than or equal to or any number in between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 amino acid residues of the HBcAg polypeptide. In some embodiments, the nucleic acid encodes a full length HBcAg polypeptide. Optionally, these sequences can be codon optimized for expression in humans. Methods of using the foregoing compositions to generate an immune response (e.g., a T cell and/or antibody specific immune response) or to treat or inhibit a HDV infection in a subject, preferably a human and, optionally a chronically infected human, are contemplated embodiments. Optionally, a subject can be identified as one in need of an immune response to HDV prior to administration of the composition and/or said subject can be evaluated for the immune response or viral clearance after administration of said compositions and such identification and/or evaluation can be accomplished using readily available diagnostics and/or clinical approaches.

In some embodiments, the self-cleavage polypeptide exists after amino acid residue number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 of the HBcAg polypeptide. Optionally, these sequences can be codon optimized for expression in humans. In some embodiments, the self-cleavage polypeptide exists before amino acid residue number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 of the HBcAg polypeptide. Optionally, these sequences can be codon optimized for expression in humans. Methods of using the foregoing compositions to generate an immune response (e.g., a T cell and/or antibody specific immune response) or to treat HDV in a subject, preferably a human and, optionally a chronically infected human, are contemplated embodiments. Optionally, a subject can be identified as one in need of an immune response to HDV prior to administration of the composition and/or said subject can be evaluated for the immune response or viral clearance after administration of said compositions and such identification and/or evaluation can be accomplished using readily available diagnostics and/or clinical approaches.

In some embodiments, the isolated nucleic acid further comprises a nucleic acid encoding a second antigenic polypeptide from HDV, for example a target antigenic polypeptide from HDAg (e.g., HDAg-L or HDAg-S). In some aspects, the second antigenic polypeptide (e.g., HDAg-L or HDAg-S) is encoded in said nucleic acid in a contiguous open reading frame. In some aspects, the second antigenic polypeptide (e.g., HDAg-L or HDAg-S) is joined to said HBcAg polypeptide or antigenic portion thereof. In some aspects, the second antigenic polypeptide (e.g., HDAg-L or HDAg-S) is joined to said self-cleavage sequence. In some aspects, the self-cleavage sequence exists within said second antigenic polypeptide (e.g., HDAg-L or HDAg-S). In some aspects, the nucleic acid encoding said HBcAg polypeptide or antigenic portion thereof and/or said second antigenic polypeptide (e.g., HDAg-L or HDAg-S) is codon optimized for expression in humans.

Some embodiments of the immunogenic composition include a nucleic acid encoding a heterologous protein or polypeptide (e.g., HDAg-L or HDAg-S), which may comprise a self-cleavage sequence. The heterologous protein or polypeptide encoded by the nucleic acid preferably comprises a hepatitis D virus (HDV) antigen, such as HDAg (e.g., nucleic acids encoding (e.g., HDAg-L or HDAg-S) (SEQ ID NOs:33, 35, 37, 39). The HDV antigens in the construct (e.g., HDAg-L or HDAg-S) can be from any isotype of HDV, which can infect humans or animals of any species. Similarly, the HBcAg sequence used in the construct, as described below, can be from any isotype of HBV, which can infect humans or animals of any species, including, but not limited to, amphibians, reptiles, birds (e.g., stork and heron), and heron, mice, hamsters, rodents, pigs, micro-pigs, goats, dogs, cats, humans and non-human primates (e.g., baboons, monkeys, and chimpanzees). In certain embodiments, the construct encoding the HBcAg with self cleavage site(s) further comprises a nucleic acid encoding an HDV antigen (e.g., HDAg-L or HDAg-S) or a fragment of a HDV polypeptide (e.g., HDAg-L or HDAg-S), wherein said fragment is at least or any number in between 30, 40, 50, 60, 70, 80, 90, or 100 amino acids in length, or wherein said fragment is at least or any number in between 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the respective full length protein. Preferably, these nucleic acids are codon optimized for expression in humans and these nucleic acids can be used as an immunogen to inhibit HDV infection or proliferation.

In certain embodiments, an expression construct comprises a nucleic acid sequence encoding a full-length HBcAg from any isotype (e.g., human, avian, or rodent), which is joined to a nucleic acid encoding a HDV polypeptide (e.g., HDAg-L or HDAg-S) and in other embodiments, the nucleic acid of the expression construct encdes an antigenic fragment of HBcAg (e.g., human, avian, or rodent) joined to a nucleic acid encoding a HDV polypeptide (e.g., HDAg-L or HDAg-S), wherein said fragments are at least or any number in between 30, 40, 50, 60, 70, 80, 90, or 100 amino acids in length, or 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the full length protein. Preferably, one or more of these nucleic acids are codon optimized for expression in humans and these nucleic acids can be used as an immunogen to inhibit HDV infection or proliferation. Methods of using the foregoing compositions to generate an immune response (e.g., a T cell and/or antibody specific immune response) or to treat HDV in a subject, preferably a human and, optionally a chronically infected human, are contemplated embodiments. Optionally, a subject can be identified as one in need of an immune response to a HDV prior to administration of the composition and/or said subject can be evaluated for the immune response or viral clearance after administration of said compositions and such identification and/or evaluation can be accomplished using readily available diagnostics and/or clinical approaches.

A number of HDV target antigenic second polypeptides are contemplated for joining to an HBcAg sequence comprising a self-cleavage site (e.g., P2A, T2A, E2A, or F2A, of or encoded by SEQ ID NOs:11-18, with or without an N-terminal GSG motif of or encoded by, for example, SEQ ID NOs:19-20). In some embodiments, the nucleic acids that are joined to the nucleic acids encoding the HBcAg (e.g., a stork or heron HBcAg) comprising a self-cleavage site (e.g., P2A, T2A, E2A, or F2A, nucleic acids of or encoding SEQ ID NOs:11-18, with or without an N-terminal GSG motif of or encoded by, for example, SEQ ID NOs:19-20) are nucleic acids encoding a hepatitis D virus antigen (HDAg) polypeptide (e.g., HDAg-L or HDAg-S), such as that of or encoded by SEQ ID NOs:33-40. In some embodiments, the second nucleic acid sequence that encodes said HDV second antigenic polypeptide (e.g., HDAg-L or HDAg-S), encodes greater than or equal to or any number in between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, or 214 amino acid residues of the HDAg. Preferably, these nucleic acid sequences are codon-optimized for expression in humans. In some embodiments, the second nucleic acid sequence that encodes said second HDV antigenic polypeptide (e.g., HDAg-L or HDAg-S), encodes greater than or equal to or any number in between 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the HDAg. Preferably, these nucleic acid sequences are codon-optimized for expression in humans. In some embodiments, the nucleic acids encode one or more self-cleavage polypeptides (e.g., P2A, T2A, E2A, or F2A, for example those of or encoded by SEQ ID NOs:11-18 with or without an N-terminal GSG motif of or encoded by, for example, SEQ ID NOs:19-20, forming, for example SEQ ID NOs:21-22) after 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the HDAg polypeptide (e.g., HDAg-L or HDAg-S). Preferably, these nucleic acid sequences are codon-optimized for expression in humans. Methods of using the foregoing compositions to generate an immune response (e.g., a T cell, such as CTL or T helper, and/or antibody specific immune response) or to treat a hepatitis D infection in a subject, preferably a human and, optionally a chronically infected human, are contemplated embodiments. Optionally, a subject can be identified as one in need of an immune response to HDV prior to administration of the composition and/or said subject can be evaluated for the immune response or viral clearance after administration of said compositions and such identification and/or evaluation can be accomplished using readily available diagnostics and/or clinical approaches.

In some embodiments, the immunogenic composition further comprises an adjuvant or an adjuvant is co-administered with said immunogenic composition or one or more of the nucleic acids described herein. In some embodiments, the adjuvant comprises a nucleic acid encoding a polypeptide adjuvant. In some embodiments, the adjuvant comprises a nucleic acid encoding a polypeptide adjuvant such as IL-12 or an adjuvant promoting polypeptide fragment thereof, IL-15 or an adjuvant promoting polypeptide fragment thereof, or IL-21 or an adjuvant promoting polypeptide fragment thereof. In some embodiments, the nucleic acid encoding the polypeptide adjuvant is codon optimized for expression in humans.

In some embodiments, the adjuvant comprises a polypeptide. In some embodiments, the adjuvant comprises a polypeptide, such as IL-12 or an adjuvant promoting polypeptide fragment thereof, IL-15 or an adjuvant promoting polypeptide fragment thereof, or IL-21 or an adjuvant promoting polypeptide fragment thereof.

In some embodiments, the adjuvant comprises a small molecule adjuvant. In some embodiments, the small molecule is ribavirin or a nucleic acid comprising one or more CpG motifs.

Some embodiments comprise an immunogenic composition comprising at least one of the isolated nucleic acid sequences discussed above, preferably codon optimized for expression in humans, for use in generating an immune response in a subject (e.g., a human) or for DNA vaccination, inoculation, or introduction into a subject so as to inhibit or treat HDV (preferably a chronic infection) or to ameliorate HDV infection or a symptom thereof. Some embodiments comprise an immunogenic composition comprising at least one of the isolated nucleic acid sequences discussed above, preferably codon optimized for expression in humans, in combination with or co-administered with an adjuvant (e.g., IL-12, IL-15, IL-21, or a nucleic acid encoding one or more of these molecules or ribavirin or a nucleic acid having one or more CpG motifs) for use in generating an immune response in a subject (e.g., a human) or for DNA vaccination, inoculation, or introduction into a subject so as to inhibit or treat HDV infection in a subject (preferably a chronic infection) or to ameliorate HDV infection or a symptom thereof. In some aspects, the HDV polypeptide (e.g., HDAg-L or HDAg-S) encoded by the nucleic acid that also comprises a sequence encoding an HBcAg sequence (e.g., an avian HBcAg such as stork or heron) that comprises a self-cleavage sequence (e.g., P2A, T2A, E2A, or F2A, with or without an N-terminal GSG motif) is a HDAg polypeptide (e.g., HDAg-L or HDAg-S) or antigenic fragment thereof (e.g., a fragment thereof, such as 30, 40, 50, 60, 70, 80, 90, or 100 amino acids in length). Preferably, these nucleic acid sequences are codon-optimized for expression in humans Optionally, steps of identifying a subject in need of an immune response to HDV, and/or evaluating the subject's response to said nucleic acid (e.g., antigen-specific antibody titer, T cell, T helper response, CTL response, or improved condition) after administration can be taken. Clinical evaluation and diagnostic tests are readily available to identify whether a subject is in need of such a therapy and/or to evaluate the subject's response to said immunogens and/or DNA immunogenic compositions or vaccines.

There is also provided a method of eliciting an immune response including administering any of the immunogenic compositions described above at a first time. In some methods, the nucleic acid or expression construct comprising one or more of the aforementioned nucleic acids is injected into a patient. In another method, the nucleic acid or said construct is administered by electroporation. In other embodiments, the method further includes administering a second immunogenic composition. In one method, the second immunogenic composition comprises an adjuvant. In another method, the second immunogenic composition is administered at a second time. In another method, the second composition includes a nucleic acid encoding an antigen of a target disease vector e.g., hepatitis D virus antigen (HDAg) polypeptide (e.g., HDAg-L or HDAg-S). In another method, the second composition also includes a nucleic acid encoding a polypeptide adjuvant.

In some methods, an adjuvant is provided in addition to said nucleic acid or construct comprising said nucleic acid and the adjuvant comprises a nucleic acid encoding a polypeptide adjuvant. In some embodiments, the adjuvant comprises a nucleic acid encoding a polypeptide adjuvant such as IL-12 or an adjuvant promoting polypeptide fragment thereof, IL-15 or an adjuvant promoting polypeptide fragment thereof, or IL-21 or an adjuvant promoting polypeptide fragment thereof. In some embodiments, the nucleic acid encoding the polypeptide adjuvant is codon optimized for expression in humans.

In other methods, the adjuvant comprises a polypeptide. In some embodiments, the adjuvant comprises a polypeptide such as IL-12 or an adjuvant promoting polypeptide fragment thereof, IL-15 or an adjuvant promoting polypeptide fragment thereof, or IL-21 or an adjuvant promoting polypeptide fragment thereof. In some aspects, the adjuvant comprises a small molecule adjuvant. In some embodiments, the small molecule is ribavirin or a nucleic acid comprising one or more CpG motifs.

In some methods, the second immunogenic composition includes a polypeptide. In one aspect, the polypeptide is derived from the same source as is the polypeptide encoded by the second antigen nucleic acid sequence. In some embodiments, a polypeptide of the second immunogenic composition includes an antigen of a hepatitis D HDAg polypeptide (e.g., HDAg-L or HDAg-S).

In some embodiments, the second time is after the first time. In another embodiment, the first time is after the second time. In other embodiments, the first time and the second time are separated by at least one, two, three, four, five, six, seven or eight weeks. In some embodiments, the number of immunizations is varied in various administration points, so that 1, 2, 3, 4, 5, or more than 5 immunogenic composition doses are administered in a first and/or a second time.

In some embodiments, the dose administered is varied from a first time to a second time. In some embodiments, the delivery strategy is varied between a first and a second introduction in the patient, for example such that a regular needle-syringe intramuscular injection is used at one time while in vivo electroporation is used at a second time, or alternatively an IVIN injection is used instead of the regular injection and/or in vivo electroporation is used. Exemplary approaches for IVIN and/or electroporation can be found in PCT Publication WO 2012/172424, designating the United States and published Dec. 20, 2012 in the English language, the contents of which are hereby expressly incorporated by reference in its entirety.

Some embodiments concern methods of ameliorating a hepatitis D infection, reducing sensitivity to HDV, or reducing HDV viral titer. By one approach, an immunogenic composition comprising a nucleic acid encoding an HBcAg (e.g., a human codon-optimized nucleic acid encoding a HBcAg derived from an avian hepatitis, such as a hepatitis that infects stork (e.g., nucleotide sequence of or encoding SEQ ID NOs:1-2) or heron (e.g., nucleotide sequence of or encoding SEQ ID NOs:3-4), an HDV polypeptide (preferably full-length, L or S HDAg polypeptide, such as that seen in or encoded by SEQ ID NOs:33-40), and a self-cleavage polypeptide (e.g., P2A, E2A, F2A, or T2A, such as that seen in or encoded by SEQ ID NOs:11-18, or 21-22, in combination with HBcAg in SEQ ID NOs:23-30) is used for ameliorating an hepatitis D infection, reducing sensitivity to HDV, or reducing HDV viral titer in an infected subject. That is, preferred compositions comprise, consist essentially of, or consist of a nucleic acid encoding an HBcAg derived from an avian hepatitis, an HDV polypeptide (e.g., HDAg-L or HDAg-S) and a self-cleavage polypeptide (e.g., P2A, E2A, F2A, or T2A). The nucleic acids present in said compositions can be in Cis (e.g., operably joined in frame) or in Trans (e.g., on separate expression constructs altogether, wherein the HBcAg and/or the HDV antigen comprises the self-cleavage sequence). By one approach, an individual in need of a medicament that ameliorates a hepatitis D infection, reduces sensitivity to HDV, or reduces HDV viral titer in an infected subject is identified (e.g., by clinical laboratory test) and said individual is provided a medicament comprising a nucleic acid encoding an avian HBcAg (e.g., SEQ ID NOs:1-4) or an HDV polypeptide (preferably full-length), and a self-cleavage polypeptide (e.g., P2A, E2A, F2A, or T2A). Preferably, these nucleic acid sequences are codon-optimized for expression in humans.

Preferred vectors or expression constructs, comprising, as illustrative examples the nucleic acids of SEQ ID NOs:31-32 and one or more of the immunogenic nucleic acid sequences disclosed herein), which can be used to inhibit or treat HDV comprise a nucleic acid encoding an HDV polypeptide (e.g., full-length HDV or HDAg-L or HDAg-S)) joined to an avian HBcAg (e.g., a stork, heron, or duck HBcAg), wherein said HBcAg comprises a self-cleavage site (e.g., P2A, E2A, F2A, or T2A with or without a GSG N-terminal motif) that separates said HBcAg from said HDV polypeptide (e.g., HDAg-L or HDAg-S) and, optionally one or more self-cleavage sites (e.g., P2A, E2A, F2A, or T2A with or without a GSG N-terminal motif) within said HBcAg. Preferably, these nucleic acid sequences encoding said fusion proteins comprising a HDV polypeptide (e.g., HDAg-L or HDAg-S), HBcAg and said self-cleavage site are codon-optimized for expression in humans. These vectors or expression constructs can be administered to subjects that have HBV and/or HDV infection so as to treat or inhibit said infections and these methods can include steps of identifying a subject having a need for an immune response to HBV, and/or HDV and/or evaluating said subject after administration of said vectors or expression constructs for T cell response (e.g., a CTL or T helper-specific response) and/or antibodies that are specific for HDV and/or HBcAg. It is preferred that these vectors or expression constructs are administered to the muscle of said subjects by IVIN injection (High pressure injection using the needles described in WO 2012/172424) and/or electroporation (e.g., IVIN with electroporation). Adjuvants including nucleic acids encoding IL12, IL15, or IL21 can be co-administered or part of the administered composition or IL12, IL15, or IL21 polypeptides can be provided.

### BRIEF DESCRIPTION OF THE DRAWINGS

FIG. 1 illustrates a variety of constructs (nucleic acids) encoding HDAg of HDV genotypes gt1 and gt2 (e.g., HDAg-L or HDAg-S of gt1 and HDAg-L or HDAg-S of gt2), which can be codon-optimized for expression in humans (constructs L1.0, L2.0, S1.0, and S2.0, for example nucleic acids of or encoding SEQ ID NOs:85-92); and, which can be joined to a nucleic acid sequence encoding an HBcAg, which can also be codon optimized for expression in humans (preferably an avian HBcAg, such as stork or heron) vis a vis a self-cleavage site, such as P2A site (constructs L1.1, L2.1, S1.1, S2.1, for example SEQ ID NOs:93-100). A third set of constructs shows a nucleic acid encoding an HBcAg, which can be codon-optimized for expression in humans, joined to a nucleic acid encoding a HDAg sequence (e.g., HDAg-L or HDAg-S), which can also be codon-optimized for expression in humans, vis a vis a self-cleavage site (e.g., a P2A site), wherein said HBcAg also comprises a plurality of self-cleavage sites (e.g., P2A) encoded by said nucleic acid within the coding sequence of HBcAg (constructs L1.2, L2.2, S1.2, and S2.2, for example nucleic acids of or encoding SEQ ID NOs:101-108). A fourth set of constructs shows a nucleic acid encoding an HDAg of HDV genotypes gt1 and gt2 (e.g., HDAg-L of genotypes gt1 or gt2 or HDAg-S of genotypes gt1 or gt2), which can be codon-optimized for expression in humans without a self-cleavage site joined to a nucleic acid encoding an HBcAg, which can be codon-optimized for expression in humans (constructs L1.3, L2.3, S1.3, and S2.3, for example nucleic acids of or encoding SEQ ID NOs:109-116). A fifth set of constructs shows HDAg coding regions of HDV genotypes gt1 and gt2 (e.g., HDAg-L or HDAg-S), which can be codon-optimized for expression in humans, into which a self-cleavage sequence has been inserted (constructs L1.4, L2.4, S1.4 and S2.4, for example nucleic acids of or encoding SEQ ID NOs:117-124). A sixth set of constructs shows HDAg coding regions of HDV genotypes gt1 and gt2 (e.g., HDAg-L or HDAg-S), which can be codon-optimized for expression in humans, into which a self-cleavage sequence has been inserted, joined without an intervening self-cleavage site such as a P2A site to a nucleic acid encoding an HBcAg, which can be codon-optimized for expression in humans (constructs L1.5, L2.5, S1.5, and S2.5, for example nucleic acids of or encoding SEQ ID NOs:125-132). A seventh set of constructs shows HDAg coding regions of HDV genotypes gt1 and gt2 (e.g., HDAg-L or HDAg-S), which can be codon-optimized for expression in humans, into which a self-cleavage sequence has been inserted, joined to a nucleic acid encoding an HBcAg sequence, which can also be codon-optimized for expression in humans, vis a vis a self-cleavage site (e.g., a P2A site) (constructs L1.6, L2.6, S1.6, and S2.6, for example nucleic acids of or encoding SEQ ID NOs:133-140). An eighth set of constructs shows HDAg coding regions of HDV genotypes gt1 and gt2 (e.g., HDAg-L of HDV genotypes gt1 and gt2 or HDAg-S of HDV genotypes gt1 and gt2), which can be codon-optimized for expression in humans, into which a self-cleavage sequence has been inserted, joined to a nucleic acid encoding an HBcAg sequence, which can also be codon-optimized for expression in humans, vis a vis a self-cleavage site (e.g., a P2A site), wherein said HBcAg also comprises a plurality of self-cleavage sites (e.g., P2A) encoded by said nucleic acid within the coding sequence of HBcAg (constructs L1.7, L2.7, S1.7, and S2.6, for example nucleic acids of or encoding SEQ ID NOs:141-148). Also contemplated herein are constructs having an HBcAg coding region interrupted by one or more self-cleavage sites such as P2A sites, wherein the order of the HBcAg fragments is shuffled as compared to the wild-type order, so that the HBcAg sequence between P2A insertions is not consecutive as to the order in an uninserted HBcAg sequence (for example, 4-3-2-1, 1-3-2-4, 2-4-1-3, and all other possible permutations of numbers as compared to the wild-type order of 1-2-3-4. Similarly, the HDAg fragments on either side of the one or more self-cleaving sites such as P2A sites inserted into the nucleic acid sequence may be shuffled in order as to the wild-type order (for example, 2-1 rather than 1-2 in the instance of a single insertion, or 1-3-2, 2-1-3, 2-3-1, 3-1-2, 3-2-1, as compared to 1-2-3 for the example where the HDAg sequence is interrupted by two P2A sequences. Some guidance as to the selection of insertion sites to preserve HDAg antigenicity is provided by Huang et al. (2004) J. Gen Vir. 85:3089-3098; Wang et al. (2007) 81(9):4438-4444; and Huang et al. (2009) J. Hepatology 50:779-788, although additional insertion sites are contemplated.

### DETAILED DESCRIPTION

It has been discovered that hepatitis B core antigen (HBcAg) is a potent adjuvant that improves the immune response of a subject to a co-administered antigen (See, e.g., PCT Publication No. WO 2010/086743 A2, published Aug. 5, 2010, which is hereby incorporated by reference in its entirety). In the present disclosure, it is contemplated that a nucleic acid encoding HBcAg interrupted by sequence encoding one or more self-cleavage polypeptide sequences fused in frame to a nucleic acid sequence encoding a HDV antigen (e.g., HDAg-L or HDAg-S) improves the immune response of a mammal to the second polypeptide antigen.

Accordingly, some embodiments include methods of enhancing or improving an immune response of a subject, wherein a nucleic acid encoding an HBcAg, one or more self-cleavage polypeptides and a HDV polypeptide (e.g., HDAg-L or HDAg-S) is provided to a subject. In some embodiments, a nucleic acid encoding a HDV polypeptide (e.g., HDAg-L or HDAg-S) is provided in Cis with a nucleic acid encoding one or more self-cleavage polypeptide sequences and a nucleic acid encoding the HBcAg (e.g., a single open reading frame encoding HBcAg joined to and in some embodiments interrupted by one or more self-cleavage sequences, see for example nucleic acids of or encoded by SEQ ID NOs:58-73). In other embodiments, the HDV peptide immunogen (e.g., HDAg-L or HDAg-S) or nucleic acid encoding a HDV peptide immunogen (e.g., HDAg-L or HDAg-S) is provided in Trans with the nucleic acid encoding the HBcAg/self-cleavage polypeptide (e.g., HBcAg or a nucleic acid encoding HBcAg, see for example SEQ ID NOs:1-10) in a mixture. Preferably, the compositions described herein comprise, consist essentially of, or consist of nucleic acid encoding “avian HBcAg,” (that is an HBcAg derived from a hepatitis virus that infects a bird, such as stork or heron) fused to or fused to and interrupted by one or more self-cleavage P2A polypeptides, optionally also including a Glycine Serine Glycine “GSG” self-cleavage enhancer polypeptide triplet. It is contemplated that the use of avian HBcAg fused to or interrupted by one or more self-cleavage P2A polypeptides in the compositions described herein will allow the formulation of immunogenic compositions that are suitable for eliciting an enhanced immune response to a second HDV polypeptide (e.g., HDAg-L or HDAg-S) also encoded by a nucleic acid. Preferably, the HDV antigen (e.g., HDAg-L or HDAg-S) and the HBcAg/P2A chimeric sequence are encoded in a single nucleic acid open reading frame and said nucleic acids are codon optimized for expression in humans.

Accordingly, one or more of the compositions described herein can be used to improve, enhance or generate an immune response to a HDV antigen (e.g., HDAg-L or HDAg-S) in a subject. By some approaches, a subject in need of an immune response to an HDV antigen (e.g., HDAg-L or HDAg-S) is identified. The identification step can be accomplished by diagnostic approaches or clinical evaluation (e.g., a subject in need of an immune response to HDV can be identified by diagnostic test or clinical evaluation). Next, one or more of the HBcAg encoding compositions described herein is provided to the identified subject. In some embodiments, the composition comprises nucleic acid encoding an HBcAg protein or fragment thereof that is at least, equal to or any number in between about 20, 30, 40, 50, 60, 70, 80, 90, 100, 150, or more amino acids (e.g., HBcAg from a hepatitis that infects birds or humans), fused to and, optionally, interrupted by at least one self-cleavage polypeptide sequences (preferably P2A self-cleavage sequences) and an antigen to which an immune response is desired (e.g., an HDV protein, such as HDAg-L or HDAg-S). Preferably, the compositions described above utilize a nucleic acid encoding an HBcAg protein that is derived from an avian hepatitis virus, such as stork or heron (e.g., SEQ ID NOs:1-4) and a P2A self-cleavage sequence. Preferably, the peptide antigens or nucleic acids encoding said peptide antigens are hepatitis D virus antigens (e.g., HDAg-L or HDAg-S). Exemplary constructs and nucleic acids encoding preferred antigens, which can be used in one or more of the compositions and methods described herein are provided in SEQ ID NOs:33-40. Optionally, any of the aforementioned approaches can further include the step of measuring the immune response of the subject before, during, and after administration of the immunogenic composition. Such measurements can be made, for example, by diagnostic evaluation of viral titer in the case of viral disease, clinical evaluation, and scratch tests as are used when evaluating the response to allergens.

Generally, the generation, enhancement, or improvement of an immune response refers to an induction of a humoral (antibody) response and/or a cellular response (T cell response). Most simply, an increase in the amount of antigen-specific antibodies (e.g., total IgG) can be seen by utilizing one or more of the embodiments described herein. Enhancement of an immune response also refers to any statistically significant change in the level of one or more immune cells (T cells, B cells, antigen-presenting cells, dendritic cells and the like) or in the activity of one or more of these immune cells (cytotoxic T lymphocyte (CTL) activity, helper T lymphocyte (Th) activity, cytokine secretion, change in profile of cytokine secretion). The skilled artisan will readily appreciate that several methods for measuring or establishing whether an immune response is generated, enhanced, or improved are available. A variety of methods for detecting the presence and levels of an immune response are available, for example. (See, e.g., Current Protocols in Immunology, Ed: John E. Coligan, et al. (2001) John Wiley & Sons, NY, N.Y.; Current Protocols in Molecular Biology, (2001), Greene Publ. Assoc. Inc. & John Wiley & Sons, NY, N.Y.; Ausubel et al. (2001) Current Protocols in Molecular Biology, Greene Publ. Assoc. Inc. & John Wiley & Sons, Inc., NY, N.Y.; Sambrook et al. (1989) Molecular Cloning, Second Ed., Cold Spring Harbor Laboratory, Plainview, N.Y.); Maniatis et al. (1982) Molecular Cloning, Cold Spring Harbor Laboratory, Plainview, N.Y.; and elsewhere). Illustrative methods useful in this context include intracellular cytokine staining (ICS), ELISPOT, proliferation assays, cytotoxic T cell assays including chromium release or equivalent assays, and gene expression analysis using any number of polymerase chain reaction (PCR) or RT-PCR based assays. For example, the number of CD8⁺ T-cells specific for a particular antigen or T-cell epitope (TCE) can be measured by flow cytometry. (See, e.g., Frelin et al. (2004) Gene Therapy 11:522-533; Holmstrom et al. (2013) J. Immunol. 190:1113-1124). CTL priming and effector function can also be measured in vivo by, for example, a tumor inhibition model, in, which the ability of an animal (e.g., mouse) to inhibit growth of tumors derived from tumor cells engineered to express the antigen of interest. Id.

In some embodiments, generation or enhancement of an immune response comprises an increase in target-specific CTL activity of between 1.5 and 5 fold in a subject that is provided a composition that comprises the nucleic acids disclosed herein (e.g., in the context of a HBcAg nucleic acid fused to and optionally interrupted by nucleic acid sequence encoding at least one self-cleavage polypeptide), wherein the TCE is derived from the target, as compared to the same TCE that is not provided in the context of the compositions disclosed herein. In some embodiments, an enhancement of an immune response comprises an increase in target-specific CTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject that is provided a composition that comprises a nucleic acid disclosed herein (e.g., in the context of a HBcAg nucleic acid or polypeptide immune response), wherein the TCE is derived from the target, as compared to administration of the same TCE that is not provided in the context of the compositions disclosed herein.

In other embodiments, an alteration of an immune response comprises an increase in target-specific Th cell activity, such as proliferation of helper T cells, of between 1.5 and 5 fold in a subject that is provided a composition that comprises a nucleic acid or polypeptide disclosed herein (e.g., in the context of a HBcAg nucleic acid or polypeptide), wherein the TCE is derived from the target, as compared to the same TCE that is not provided in the context of the compositions disclosed herein. In some embodiments, alteration of an immune response comprises an increase in target-specific Th cell activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject that is provided a composition that comprises a nucleic acid disclosed herein (e.g., in the context of a HBcAg nucleic acid), wherein the TCE is derived from the target, as compared to administration of the same TCE that is not provided in the context of the compositions disclosed herein. In this context, an enhancement in Th cell activity may comprise an increase as described above, or decrease, in production of a particular cytokine, such as interferon-gamma (IFNγ), interleukin-1 (IL-1), IL-2, IL-3, IL-6, IL-7, IL-12, IL-15, IL-21, tumor necrosis factor-alpha (TNFα), granulocyte macrophage colony-stimulating factor (GM-CSF), granulocyte-colony stimulating factor (G-CSF), or other cytokine. In this regard, generation or enhancement of an immune response may comprise a shift from a Th2 type response to a Th1 type response or in certain embodiments a shift from a Th1 type response, to a Th2 type response. In other embodiments, the generation or enhancement of an immune response may comprise the stimulation of a predominantly Th1 or a Th2 type response.

In still more embodiments, an increase in the amount of antibody specific for the antigen (e.g., total IgG) is increased. Some embodiments, for example, generate an increase in second antigenic target-specific antibody production of between 1.5, 2, 3, 4, or 5 fold in a subject that is provided a composition comprising the nucleic acids or polypeptides disclosed herein, (e.g., in the context of a HBcAg nucleic acid or polypeptide), wherein the TCE is derived from the target, as compared to the same TCE that is not present in the context of the compositions disclosed herein. In some embodiments, the increase in second antigenic target-specific antibody production is about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject that is provided a composition that comprises a nucleic acid or polypeptide disclosed herein, (e.g., in the context of a HBcAg nucleic acid or polypeptide), wherein the TCE is derived from the target, as compared to as compared to administration of the same TCE that is not present in the context of the compositions disclosed herein.

Generation or enhancement of a cellular immune response can also refer to the frequency of cytotoxic T lymphocytes (CTLs) specific for a desired antigen that are primed, or the rapidity of priming of cytotoxic T lymphocytes (CTLs) specific for a desired antigen, compared to the priming of CTLs specific for the desired epitope when the epitope is not presented in the context of the nucleic acids or peptides disclosed herein. The section below describes several of the HBcAg and HDV antigenic protein sequences that can be used in the compositions and methods described herein.

Isolated Nucleic Acids and Proteins. Disclosed herein are compositions that comprise isolated nucleic acids encoding HBcAg, or a fragment thereof, joined to (e.g., flanking or juxtaposed to) an isolated nucleic acid encoding a HDV antigenic polypeptide (e.g., HDAg-L or HDAg-S). Accordingly, the isolated nucleic acid may, in some embodiments, encode a fusion protein that includes at least a fragment of HBcAg, and a second HDV antigenic protein (e.g., HDAg-L or HDAg-S). Polypeptides encoded by said isolated nucleic acids are also embodiments of the present invention.

The nucleocapsid or core antigen HBcAg of HBV is an immunogenic particle composed of 180 subunits of a single protein chain. HBcAg has been disclosed as an immunogenic moiety that stimulates the T cell response of an immunized host animal. See, e.g, U.S. Pat. No. 4,818,527, U.S. Pat. No. 4,882,145 and U.S. Pat. No. 5,143,726, each of which is hereby incorporated by reference in their entirety. It can be used as a carrier for several peptidic epitopes covalently linked by genetic engineering as well as for chemically coupled protein antigens. (See Sallberg et al. (1998) Human Gene Therapy 9:1719-29). In addition, HBcAg is non-cytotoxic in humans. Accordingly, it was contemplated that HBcAg is useful in genetic constructs for generating or enhancing an immune response to an accompanied target antigen (e.g., in constructs that encode a TCE derived from a pathogen).

Current listings of exemplary HBcAg sequences are publicly available at the National Center for Biotechnology Information (NCBI) world-wide web site. HBcAg nucleic acid sequences (including novel HBcAg regions) can also be isolated from subjects (e.g., humans) infected with HBV. DNA obtained from a patient infected with HBV can be amplified using PCR or another amplification technique.

For a review of PCR technology, see Molecular Cloning to Genetic Engineering White, B. A. Ed. in Methods in Molecular Biology 67: Humana Press, Totowa (1997) and the publication entitled “PCR Methods and Applications” (1991, Cold Spring Harbor Laboratory Press). For amplification of mRNAs, it is within the scope of the invention to reverse transcribe mRNA into cDNA followed by PCR (RT-PCR); or, to use a single enzyme for both steps as described in U.S. Pat. No. 5,322,770. Another technique involves the use of Reverse Transcriptase Asymmetric Gap Ligase Chain Reaction (RT-AGLCR), as described by Marshall R. L. et al. (PCR Methods and Applications 4:80-84, 1994).

The source of the HBcAg sequences that are included in the isolated nucleic acids described herein is not particularly limited. Accordingly, embodiments described herein may utilize an isolated nucleic acid that encodes an HBcAg derived from a hepatitis virus capable of infecting animals of any species, including but limited to, humans, non-human primates (e.g., baboons, monkeys, and chimpanzees), rodents, mice, reptiles, birds (e.g., stork and heron), pigs, micro-pigs, goats, dogs and cats. In some embodiments, the HBcAg is selected from a human hepatitis antigen or an avian hepatitis antigen. Particularly preferred are the stork hepatitis antigen and a heron hepatitis antigen.

In certain embodiments, the HBcAg sequences described herein have variations in nucleotide and/or amino acid sequences, compared to native HBcAg sequences and are referred to as HBcAg variants or mutants. As used herein, the term “native” refers to naturally occurring HBV sequences (e.g., available HBV isotypes). Variants may include a substitution, deletion, mutation or insertion of one or more nucleotides, amino acids, or codons encoding the HBcAg sequence, which may result in a change in the amino acid sequence of the HBcAg polypeptide, as compared with the native sequence. Variants or mutants can be engineered, for example, using any of the techniques and guidelines for conservative and non-conservative mutations set forth, for instance, in U.S. Pat. No. 5,364,934, which is hereby incorporated by reference in its entirety.

Accordingly, when the term “consisting essentially of” is used, in some contexts, variants or mutants of an HBcAg sequence or of a particular antigen sequence are intended to be encompassed. That is, in some contexts and in some embodiments, the variants or mutants of the sequences disclosed herein are equivalents because the variation or mutation in sequence does not change or materially affect the basic and novel characteristics of the claimed invention.

A codon-optimized HBcAg can, in some embodiments, be encoded within the isolated nucleic acid. A codon-optimized sequence may, in some embodiments, be obtained by substituting codons in an existing sequence with codons more frequently used in the intended host subject (e.g., a human). Some examples include, but are not limited to, codon-optimized nucleic acids encoding human HBcAg (e.g., SEQ ID NOs: 5, 7, 9), codon-optimized nucleic acids encoding stork HBcAg (e.g., SEQ ID NO:1), and codon-optimized nucleic acids encoding heron HBcAg (e.g., SEQ ID NO:3).

The isolated nucleic acids can encode the full-length HBcAg in certain embodiments (e.g., SEQ ID NOs:1-10), such as, for example, those sequences that correspond to residues 1-183 of a human HBcAg. However, fragments of the HBcAg may also be encoded with the nucleic acid in certain embodiments. A fragment of the HBcAg sequence that can be used in the embodiments described herein can comprise at least, equal to, greater than, or less than, or any number in between 3, 5, 10, 20, 50, 75, 100, 125, 150, or 175 consecutive amino acids of a natural or synthetic HBcAg polypeptide (e.g., a naturally occurring isotype or a codon-optimized or otherwise modified HBcAg polypeptide).

Some embodiments include, for example, one or more of the HBcAg nucleic acid or protein sequences disclosed in International Patent Application Publication Number WO 20091130588, published Dec. 7, 2011, which designated the United States and was published in English, the disclosure of which is hereby expressly incorporated by reference in its entirety.

The isolated nucleic acids further encode at least one self-cleavage polypeptide sequence. Self-cleaving 2A polypeptide sequences, also referred to herein as self-cleavage sequences, sites or domains were first identified in the food-and-mouth disease virus (Ryan, M D et al. (1991) “Cleavage of foot and mouth disease virus protein is mediated by residues located within a 19 amino acid sequence.” J. Gen. Virol. 72(Pt 11):2727-2732). The ‘cleavage’ of a 2A peptide from its immediate downstream peptide is in fact affected by ribosomal skipping of the synthesis of the glycyl-prolyl peptide bond at the C-terminus of the 2A polypeptide (Lyan Lab Webpage; de Felipe P, Luke G A, Brown J D, Ryan M D (2010) Inhibition of 2A-mediated ‘cleavage’ of certain artificial polyprotein bearing N-terminal signal sequences. Biotechnol J 5: 213-223; Donnelly M L, Luke G, Mehrotra A, Li X, Hughes L E, et al. (2001) Analysis of the aphthovirus 2A/2B polyprotein ‘cleavage’ mechanism indicates not a proteolytic reaction, but a novel translational effect: a putative ribosomal ‘skip’. J Gen Virol 82: 1013-1025). Several 2A self-cleavage polypeptides have been isolated (see, e.g., Szymczak A L, Vignali D A (2005) Development of 2A peptide-based strategies in the design of multicistronic vectors. Expert Opin Biol Ther 5: 627-638, the disclosure of which is hereby incorporated by reference in its entirety). Four of the 2A polypeptide sequences identified to date have seen substantial use in biomedical research: picornavirus 2A sequences FMDV 2A (abbreviated herein as F2A); equine rhinitis A virus (ERAV) 2A (E2A); porcine teschovirus-1 2A (P2A), and insect virus Thosea asigna virus 2A (T2A), (de Felipe P, Luke G A, Hughes L E, Gani D, Halpin C, et al. (2006) E unum pluribus: multiple proteins from a self-processing polyprotein. Trends Biotechnol 24: 68-75).

Self-cleaving 2A sequences are preferred over alternative methods of expressing multiple proteins from a single construct, such as Internal Ribosomal Entry Sequences (IRES), because of their short length and stoichiometric expression of multiple proteins flanking the 2A polypeptide (de Felipe P, Luke G A, Hughes L E, Gani D, Halpin C, et al. (2006) E unum pluribus: multiple proteins from a self-processing polyprotein. Trends Biotechnol 24: 68-75).

Recent results indicate that P2A self-cleavage polypeptides demonstrate the highest cleavage efficiency of the self-cleavage polypeptides in regular use (See, e.g., Kim J H et al., (2011) High Cleavage Efficiency of a 2A Peptide Derived from Porcine Teschovirus-1 in Human Cell Lines, Zebrafish and Mice, PLoS ONE 6(4):1-8, e18556). Accordingly, P2A, such as the P2A sequence of SEQ ID NO:11-12 is a preferred self-cleavage sequence of some embodiments disclosed herein. However, other self-cleavage or self-cleavage polypeptides are contemplated, and the disclosure herein is not limited to a single self-cleavage sequence (See, e.g., Gao et al., (2012) Towards Optimising the Production of and Expression from Polycistronic Vectors in Embryonic Stem Cells, PLoS ONE 7(11):1-13., e48668.

Results indicate that the addition of a Glycine-Serine-Glycine (“GSG”) leader immediately preceding a self-cleavage polypeptide such as P2A may increase the ‘cleavage’ efficiency in some embodiments (See, e.g., Lorens J, Pearsall D M, Swift S E, Peelle B, Armstrong R, et al. (2004) Stable, stoichiometric delivery of diverse protein functions. J Biochem Biophys Method 58: 101-110). Accordingly, in some embodiments a GSG leader precedes a self-cleavage polypeptide such as P2A, as seen for example in SEQ ID NO:21-22.

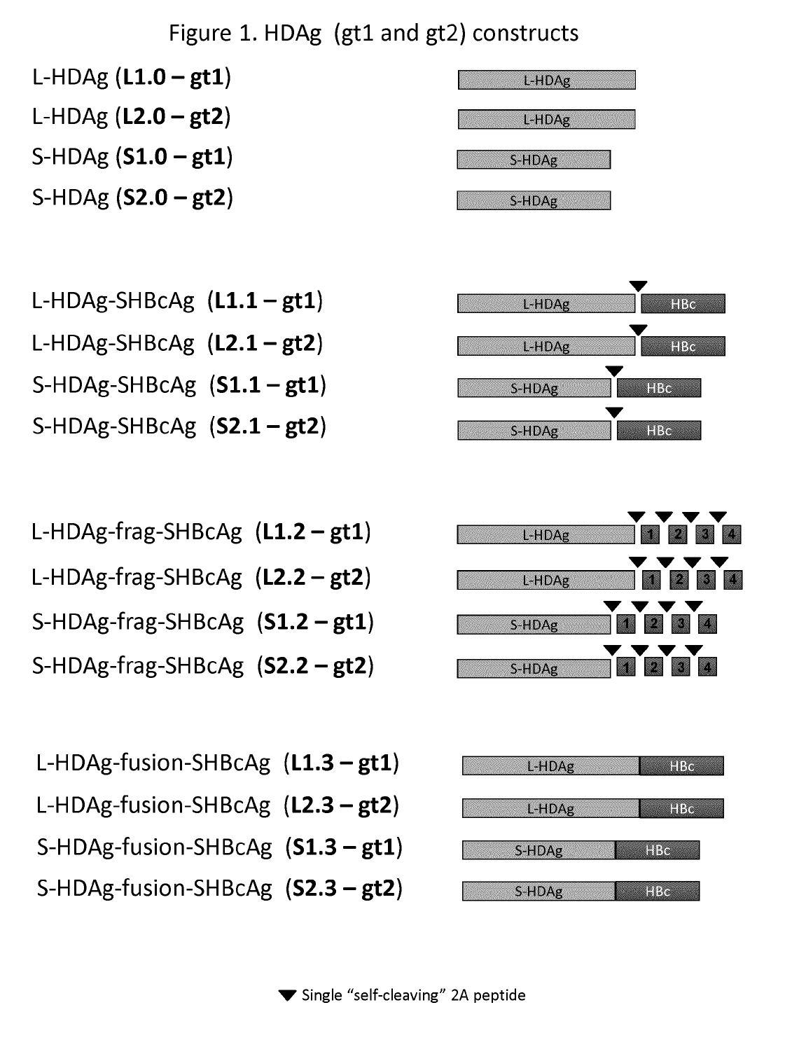

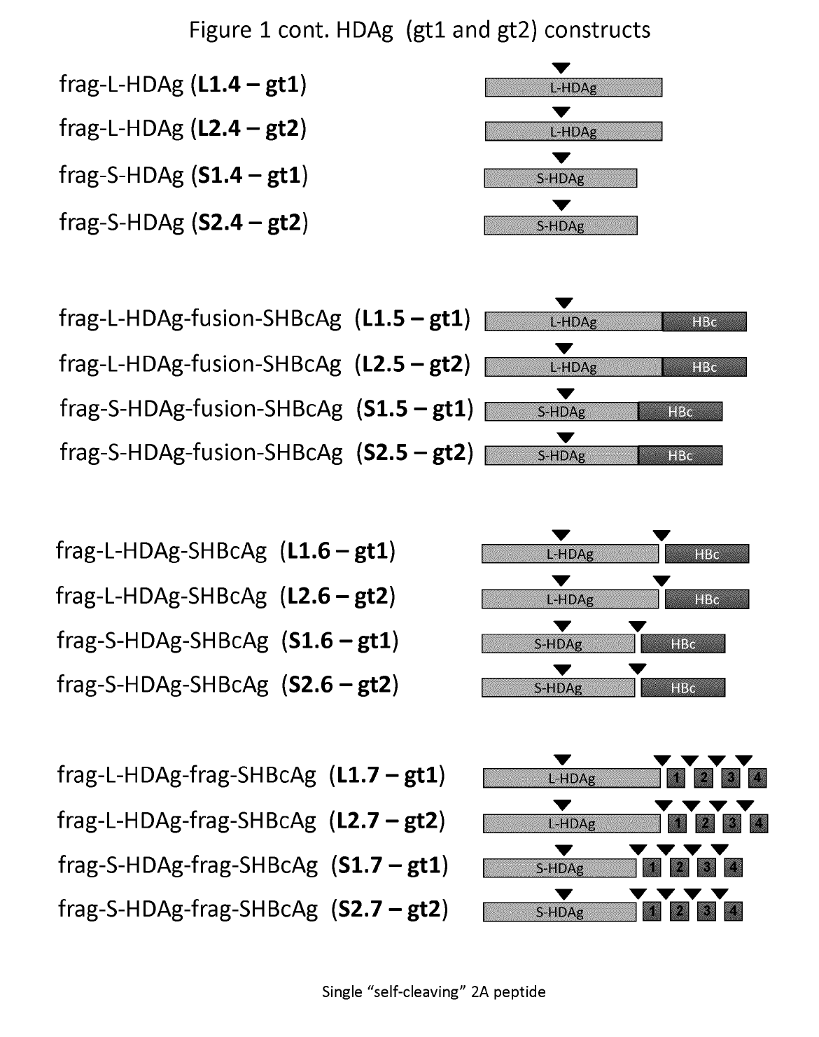

Disclosed herein are constructs comprising a nucleic acid encoding HBcAg interrupted by a sequence encoding one or more self-cleavage polypeptide sequences, fused in frame to a nucleic acid sequence encoding a HDV polypeptide antigen (e.g., HDAg-L or HDAg-S), which will improve the immune response of a mammal to the HDV polypeptide antigen (e.g., HDAg-L or HDAg-S), as compared to a nucleic acid construct encoding the HBcAg fused to the HDV polypeptide antigen (e.g., HDAg-L or HDAg-S) but lacking said self-cleavage sequences. See, for example FIG. 1, which illustrates several contemplated constructs that can be used in the methods described herein (e.g., to treat and/or inhibit HDV infection). The embodiments contemplated herein are not however limited to a specific self cleavage site existing at a specific location within an encoded HBcAg polypeptide sequence. For example, a first, or a first and a second, or a first and a second and a third, or a first and a second and a third and a fourth, or a first and a second and a third and a fourth and a fifth, or more than five self-cleavage sites may exist, each located at a position before or after amino acid residue number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 of the HBcAg polypeptide. In some embodiments, see FIG. 1, for example, the nucleic acid encoding the HDV polypeptide antigen (e.g., HDAg-L or HDAg-S) also encodes a plurality of self-cleavage sites within said HDV polypeptide antigen (e.g., HDAg-L or HDAg-S) and the resulting fragments of the HDV polypeptide antigen (e.g., HDAg-L or HDAg-S) are shuffled such that the fragments do not exist in a naturally occurring sequence (e.g., the epitopes present in the resultant polypeptide are presented in a non-native or non-naturally occurring order).

Meanwhile, the isolated nucleic acid encoding HBcAg (such as nucleic acids of or encoding SEQ ID NOs:1-10) may also be joined, for example via nucleic acid sequence encoding a P2A polypeptide, to an isolated nucleic acid encoding an HDV antigen (e.g., HDAg-L or HDAg-S). The HDV antigen (e.g., HDAg-L or HDAg-S) may generally vary in the same manner discussed above with respect to the HBcAg. Thus, in some embodiments, the isolated nucleic acid sequences may encode native, variants or mutants of a HDV polypeptide (e.g., HDAg-L or HDAg-S), and these nucleic acids may also be codon-optimized (e.g., a codon-optimized nucleic acid encoding HDAg from the human hepatitis virus in SEQ ID NOs:33, 35, 37, or 39). In some embodiments, the isolated nucleic acid encodes a fragment of the HDV protein (e.g., HDAg-S). In some embodiments, the HDV polypeptide is HDAg polypeptide (e.g., HDAg-L or HDAg-S) joined in Cis to a nucleic acid sequence encoding a self-cleavage sequence, which is also joined in Cis to a nucleic acid sequence encoding HBcAg. In some embodiments, the nucleic acid encoding the HDV polypeptide (e.g., HDAg-L or HDAg-S) is interrupted by one, two three, four five or more than five nucleic acid sequences encoding self-cleavage polypeptides before, at or after embodiments the nucleic acid encoding the HDV polypeptide encodes 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the full length HDV polypeptide or of the total fraction of the second polypeptide comprising an antigen (e.g., HDAg-L or HDAg-S) that is encoded in the isolated nucleic acid as disclosed herein. In some embodiments, all of the sequences include a Kozak sequence (e.g., SEQ ID NO:41) at the 5′ end of the open reading frame, optionally, comprising additional nucleotides inserted to preserve the reading frame of the encoded polypeptide, as seen in SEQ ID NOs: 42-57). In some embodiments, the sequences are cloned into an expression vector such as that of SEQ ID NOs:31-32, though other expression vectors are compatible with the compositions and methods herein. In some embodiments, said cloning may comprise additional restriction endonuclease sequence, such as that of SEQ ID NOs:80-84, for example.

The HDV antigenic protein, in some embodiments, is HDAg polypeptide antigen (e.g., HDAg-L or HDAg-S). Additional polypeptide antigens are contemplated and the disclosure is not limited as to the HDV polypeptide antigen to be encoded by a nucleic acid in Cis or transformed in Trans with an nucleic acid encoding HBcAg and one or more self-cleavage polypeptides, as disclosed herein. Some embodiments include, for example, one or more second antigenic proteins, or isolated nucleic acids encoding the same, in International Patent Application Publication Number WO 20091130588, which designated the United States and was published in English, the disclosure of which is hereby expressly incorporated by reference in its entirety.

Non-limiting examples of isolated nucleic acids encoding HBcAg (such as nucleic acids of or encoding SEQ ID NOs:1-10), or a fragment thereof, joined to an isolated nucleic acid encoding a second antigenic protein (e.g., HDAg-L or HDAg-S) by a nucleic acid encoding a self-cleavage polypeptide and, optionally, further comprising nucleic acid encoding one or more additional self-cleavage polypeptides existing within the HBcAg coding region and, optionally, further comprising a nucleic acid encoding one or more additional self-cleavage polypeptides existing within the coding region of the second polypeptide (e.g., HDAg-L or HDAg-S) include, but are not limited to: stork HBcAg (SEQ ID NOs:1-2) joined to HDAg (e.g., HDAg-L or HDAg-S); and heron HBcAg (nucleic acids of or encoding SEQ ID NOs:3-4) joined to HDAg (e.g., HDAg-L or HDAg-S). Embodiments of the isolated nucleic acids and expression constructs comprise a nucleic acid encoding HBcAg (such as, nucleic acids of or encoding SEQ ID NOs:1-10) and a plurality of isolated nucleic acids encoding HDAg polypeptides (e.g., HDAg-L or HDAg-S and fragments thereof greater than or equal to or any number in between 30, 40, 50, 60, 70, 80, 90, or 100 amino acids in length), wherein each of the isolated nucleic acids being joined together and having self-cleavage site in between.

As would be appreciated by a person of ordinary skill, the proteins encoded in the isolated nucleic acids disclosed herein may be obtained using known methods. As an example, the nucleic acids may be inserted into an appropriate plasmid, which is subsequently inserted into to cells that express the protein. Other methods for obtaining the encoded proteins are also known. Accordingly, the scope of the present application includes the proteins that can be obtained from the isolated nucleic acids disclosed herein. For example, SEQ ID NO:2 can be derived from SEQ ID NO:1. Similarly, additional nucleic acid sequence scan be derived that encode SEQ ID NO:2. Thus, embodiments of the present invention also include, but are not limited to, proteins having the sequences of the nucleic acids disclosed herein and alternate nucleic acid sequences encoding the disclosed protein sequences.

Immunogenic Compositions Comprising Nucleic Acids.

Disclosed herein are also immunogenic compositions relating to genetic constructs that include nucleic acids encoding HBcAg (such as nucleic acids of or encoding SEQ ID NOs:1-10), or a fragment thereof, and one or more self-cleavage polypeptides, and nucleic acids encoding a second antigenic protein. In some embodiments, all of the polypeptide sequences are encoded in a single open reading frame in the same nucleic acid construct (e.g., the same plasmid). In certain embodiments, the nucleic acid encoding the HDV antigenic polypeptide (e.g., HDAg-L or HDAg-S) is in a separate open reading frame or in a second nucleic acid construct. Some embodiments of the immunogenic compositions disclosed herein include one or more proteins encoded by a nucleic acid described herein.

The source of the HBcAg (such as nucleic acids of or encoding SEQ ID NOs:1-10) that is encoded in the nucleic acid is not particularly limited. Accordingly, the nucleic acid contemplated for the immunogenic compositions described herein can be nucleic acids from viruses known to infect animals of any species, including but limited to, humans, mice, reptiles, birds (e.g., stork and heron), rodents, pigs, micro-pigs, goats, dogs, cats, and non-human primates (e.g., baboons, monkeys, and chimpanzees), as mentioned above. In some embodiments, the HBcAg is selected from a human hepatitis antigen, an avian hepatitis antigen, a stork hepatitis antigen, and a heron hepatitis antigen.

The sequences encoding HBcAg (such as nucleic acids of or encoding SEQ ID NOs:1-10) and the self-cleavage 2A polypeptide or self-cleavage site can generally be the same as those discussed above with respect to the isolated nucleic acids. Thus, in some embodiments, any of the nucleic acid sequences described above that include HBcAg and/or sequence encoding the self-cleavage polypeptide may be used in the immunogenic composition. As an example, the isolated nucleic acid may include native or variant HBcAg or mutant HBcAg, and self-cleavage 2A polypeptides P2A, E2A, F2A, or T2A, or other 2A polypeptides, and the nucleic acid may also be codon-optimized for expression in humans. In some embodiments, the isolated nucleic acid encodes a fragment of HBcAg, as described above with respect to the isolated nucleic acids. For example, fragment of the HBcAg sequence can comprise at least, equal to, greater than, or less than, or any number in between 3, 5, 10, 20, 50, 75, 100, 125, 150, or 175 consecutive amino acids of a natural or synthetic HBcAg polypeptide. A full-length HBcAg can also be encoded in an isolated nucleic acid included within the immunogenic composition.

Some embodiments include nucleic acids that have homology or sequence identity to any one of the nucleic acid or polypeptide sequences encoding the fusion proteins disclosed herein (e.g., nucleic acids encoding an HBcAg, preferably an avian HBcAg, joined to an HDAg such as HDAg-L or HDAg-S having one or more self-cleavage sites, preferably P2A or polypeptides encoded by said nucleic acids) (See e.g., SEQ ID NOs:1-10, 11-18, 21-30, 33-40). In some embodiments, said homologous nucleic acids generate, enhance, or improve an immune response, as defined above. Several techniques exist to determine nucleic acid or protein sequence homology. Thus, embodiments of the nucleic acids can have from 70% homology or sequence identity to 100% homology or sequence identity to any one of the nucleic acid sequences or protein sequences disclosed herein. That is, embodiments can have at least, equal to or any number between about 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96.0%, 97.0%, 98.0%, 99.0%, and 100.0% homology or sequence identity to any one of the polypeptide or nucleic acid sequences disclosed herein.

Several homology or sequence identity searching programs based on nucleic acid sequences are known in the art and can be used to identify molecules that are homologous. In one approach, a percent sequence identity can be determined by standard methods that are commonly used to compare the similarity and position of the base pairs of two nucleic acids. Using a computer program such as BLAST or FASTA, two sequences can be aligned for optimal matching of their respective base pairs (either along the full length of one or both sequences, or along a predetermined portion of one or both sequences). Such programs provide “default” opening penalty and a “default” gap penalty, and a scoring matrix such as PAM 250 (a standard scoring matrix; see Dayhoff et al., in: Atlas of Protein Sequence and Structure, Vol. 5, Supp. 3 (1978)) can be used in conjunction with the computer program.

Some embodiments include isolated nucleic acids having sufficient homology or sequence identity to any one of the nucleic acid sequences disclosed herein (e.g., nucleic acids encoding an HBcAg, preferably an avian HBcAg, joined to an HDAg such as HDAg-L or HDAg-S having one or more self-cleavage sites, preferably P2A) such that hybridization will occur between the isolated nucleic acid and any one of the nucleic acids sequences disclosed herein. In some aspects, hybridization occurs under usual washing conditions in Southern hybridization, that is, at a salt concentration corresponding to 0.1 times saline sodium citrate (SSC) and 0.1% SDS at 37° C. (low stringency), preferably 0.1 times SSC and 0.1% SDS at 60° C. (medium stringency), and more preferably 0.1 times SSC and 0.1% SDS at 65° C. (high stringency). In certain aspects, the nucleic acid embodiments have a percentage of consecutive bases that hybridize under stringent conditions with any one of the nucleic acids sequences disclosed herein (e.g., nucleic acids encoding an HBcAg, preferably an avian HBcAg, joined to an HDAg such as HDAg-L or HDAg-S having one or more self-cleavage sites, preferably P2A or polypeptides encoded by said nucleic acids), wherein the sequence identity is greater than or equal to or any number in between 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96.0%, 97.0%, 98.0%, 99.0%, and 100.0% of the total number of bases in the nucleic acid sequence.

Some embodiments include a nucleic acid encoding a HDV protein (e.g., HDAg-L or HDAg-S) or an antigenic fragment thereof. The HDV protein (e.g., HDAg-L or HDAg-S) encoded by the nucleic acid, in some embodiments, can be from any known hepatitis D virus and can comprise full length or antigenic fragments of HDAg or the nucleic acid encoding said HDV polypeptide, preferably codon optimized for expression in humans, can have a sequence identity to said HDV protein (e.g., HDAg-L or HDAg-S) that is greater than or equal to or any number in between 40.0%, 41.0%, 42.0%, 43.0%, 44.0%, 45.0%, 46.0%, 47.0%, 48.0%, 49.0%, 50.0%, 51.0%, 52.0%, 53.0%, 54.0%, 55.0%, 56.0%, 57.0%, 58.0%, 59.0%, 60.0%, 61.0%, 62.0%, 63.0%, 64.0%, 65.0%, 66.0%, 67.0%, 68.0%, 69.0%, 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96.0%, 97.0%, 98.0%, 99.0%, and 100.0% of the total number of bases in the nucleic acid sequence.

Non-limiting examples of mixtures of nucleic acid sequences encoding HBcAg (such as nucleic acids of or encoding SEQ ID NOs:1-10), or a fragment thereof, and nucleic acid sequences encoding a HDV antigenic protein (e.g., HDAg-L or HDAg-S), that may be included in the immunogenic compositions, include, but are not limited to, nucleic acid sequences encoding: stork HBcAg (e.g., nucleic acids of or encoding SEQ ID NOs:1-2) joined to HDAg (e.g., HDAg-L or HDAg-S); and heron HBcAg (nucleic acids of or encoding SEQ ID NOs:3-4) joined to HDAg (e.g., HDAg-L or HDAg-S).

Some embodiments of the immunogenic composition include the isolated nucleic acids described above, wherein the nucleic acid encoding HBcAg (such as nucleic acids of or encoding SEQ ID NOs:1-10), or a fragment thereof, is joined to nucleic acid sequences encoding a HDV protein (e.g., HDAg-L or HDAg-S). Accordingly, further exemplary compositions may include a nucleic acid encoding: stork HBcAg (e.g., nucleic acids of or encoding SEQ ID NOs:1-2) joined to HDAg (e.g., HDAg-L or HDAg-S); and heron HBcAg (nucleic acids of or encoding SEQ ID NOs:3-4) joined to HDAg (e.g., HDAg-L or HDAg-S).

It is contemplated that various other compounds may be included in one or more of the compositions. Some embodiments of the composition may further include an additional adjuvant. Non-limiting example of adjuvants that can be included are: interleukin-2 (IL2), interleukin-12 (IL12), interleukin-15 (IL15), interleukin-21 (IL21), interleukin-28b (IL28b), galactosyl transferase, a toll-like receptor (TLR), ribavirin, alum, CpGs, or an oil. In some embodiments, the composition includes an isolated nucleic acid, or constructs comprising said nucleic acids, (preferably codon optimized for expression in humans) encoding a protein that is an adjuvant, such as IL2, IL12, IL15, IL21, IL28b, galactose transferase, a TLR, and the like. In certain aspects, the isolated nucleic acid encoding the protein, which is an adjuvant, may be in the same construct encoding HBcAg and/or the second antigenic protein. In other aspects, the isolated nucleic acid encoding the protein, which is an adjuvant, may be in a different construct than the construct encoding HBcAg and/or the second antigenic protein. In some aspects the full length protein or nucleic acid encoding the full length protein is included. In some embodiments, an adjuvant fragment polypeptide or nucleic acid encoding an adjuvant fragment polypeptide comprising 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the full length protein or nucleic acid encoding the full length protein is included.

The compositions described herein may also contain other ingredients or compounds in addition to nucleic acids and/or polypeptides, including, but not limited to, various other peptides, adjuvants, binding agents, excipients such as stabilizers (to promote long term storage), emulsifiers, thickening agents, salts, preservatives, solvents, dispersion media, coatings, antibacterial and antifungal agents, isotonic and absorption delaying agents and the like. See e.g., U.S. application Ser. No. 09/929,955 and U.S. application Ser. No. 09/930,591. These compositions are suitable for treatment of animals, particularly mammals, either as a preventive measure to avoid a disease or condition or as a therapeutic to treat animals already afflicted with a disease or condition.

Many other ingredients may also be present in the compositions provided herein. For example, the adjuvant and antigen can be employed in admixture with conventional excipients (e.g., pharmaceutically acceptable organic or inorganic carrier substances suitable for parenteral, enteral (e.g., oral) or topical application that do not deleteriously react with the therapeutic ingredients (e.g., construct encoding HBcAg (such as nucleic acids of or encoding SEQ ID NOs:1-10). Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable carriers are described in Remmington's Pharmaceutical Sciences, 15th Edition, Easton:Mack Publishing Company, pages 1405-1412 and 1461-1487(1975) and The National Formulary XIV, 14th Edition, Washington, American Pharmaceutical Association (1975).

Immunogenic Compositions Comprising Polypeptides.

Some of the embodiments described herein concern compositions that comprise, consist essentially of, or consist of polypeptides encoded by any of the nucleic acids disclosed herein. In some embodiments, the composition includes an admixture of HBcAg (such as polypeptides of or encoded by SEQ ID NOs:1-10), or a fragment thereof, and a HDV protein (e.g., HDAg-L or HDAg-S). In certain aspects, the composition includes a protein having HBcAg joined to an HDV polypeptide (e.g., HDAg-L or HDAg-S) further comprising at least one self cleavage site, such as between said HBcAg and said HDV polypeptide and/or within said HBcAg sequence, wherein said self cleavage site can be any one or more of E2A, F2A, T2A, or P2A.

The HBcAg polypeptides that may be included in the immunogenic compositions can be any HBcAg polypeptide that can be encoded in the nucleic acids within the immunogenic composition of nucleic acids discussed above, or those encoded in the isolated nucleic acids discussed above. Thus, in some embodiments, the HBcAg is derived from a codon-optimized nucleic acid. The HBcAg may also be a native or variant form of the protein. Also, the composition may include a fragment of HBcAg. A fragment of HBcAg can comprise at least, equal to, greater than, or less than, or any number in between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 consecutive amino acid residues of a natural or synthetic HBcAg polypeptide (e.g., a naturally occurring isotype or a codon-optimized or otherwise modified HBcAg polypeptide).

Some embodiments include polypeptides that have homology or sequence identity to any one of the polypeptide sequences disclosed herein (e.g., an HBcAg, preferably an avian HBcAg, joined to an HDAg such as HDAg-L or HDAg-S having one or more self-cleavage sites, preferably P2A). In some embodiments, said polypeptides generate, enhance, or improve an immune response, as defined above. Several techniques exist to determine protein sequence homology or sequence identity. Thus, embodiments of the polypeptides can have from 70% homology to 100% homology or sequence identity to any one of the polypeptides disclosed herein. That is, embodiments can have at least, equal to, or any number in between about 70.0%, 71.0%, 72.0%, 73.0%, 74.0%, 75.0%, 76.0%, 77.0%, 78.0%, 79.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 96.0%, 97.0%, 98.0%, 99.0%, and 100.0% homology or sequence identity to any one of the polypeptide or nucleic acid sequences disclosed herein (e.g., nucleic acids encoding an HBcAg, preferably an avian HBcAg, joined to an HDAg such as HDAg-L or HDAg-S having one or more self-cleavage sites, preferably P2A or polypeptides encoded by said nucleic acids).

Several homology or sequence identity searching programs based on polypeptide sequences are known in the art and can be used to identify molecules that are homologous. In one approach, a percent sequence identity can be determined by standard methods that are commonly used to compare the similarity and position of the amino acids of two polypeptides. Using a computer program such as BLAST or FASTA, two sequences can be aligned for optimal matching of their respective amino acids (either along the full length of one or both sequences, or along a predetermined portion of one or both sequences). Such programs provide “default” opening penalty and a “default” gap penalty, and a scoring matrix such as PAM 250 (a standard scoring matrix; see Dayhoff et al., in: Atlas of Protein Sequence and Structure, Vol. 5, Supp. 3 (1978)) can be used in conjunction with the computer program.

Non-limiting examples of admixtures of HBcAg, or a fragment thereof, and a second antigenic protein (e.g., HDAg-L or HDAg-S), which may be included in the immunogenic compositions, include, but are not limited to: stork HBcAg (e.g., nucleic acids of or encoding SEQ ID NOs:1-2) joined to HDAg (e.g., HDAg-L or HDAg-S); and heron HBcAg (e.g., nucleic acids of or encoding SEQ ID NOs:3-4) joined to HDAg (e.g., HDAg-L or HDAg-S).

It is also contemplated that some immunogenic compositions can comprise both a protein as described herein and a nucleic acid, as described herein. For example, some embodiments may include a nucleic acid encoding an HBcAg (e.g., a nucleic acid encoding a stork or heron HBcAg (polypeptides of or encoded by SEQ ID NOs:1-4) and a protein that is an antigen (e.g., an HDV polypeptide, such as HDAg-L or HDAg-S). Alternatively, some embodiments are immunogenic compositions that comprise an HBcAg protein (e.g., stork or heron HBcAg) and a nucleic acid encoding an antigen (e.g., an HDV polypeptide, such as HDAg-L or HDAg-S).

It is also contemplated that various other ingredients may be included to improve the immunogenic composition by, for example, increasing the immune response caused by the composition. Some embodiments of the composition may further include an adjuvant. Non-limiting example of adjuvants include interleukin-2 (IL2), interleukin-12 (IL12), interleukin-15 (IL15), interleukin-21 (IL21), interleukin-28b (IL28b), galactosyl transferase, a toll-like receptor (TLR), ribavirin, alum, CpGs, and an oil.

Various ingredients, such as excipients, adjuvants, binding agents, etc., may be included in the immunogenic compositions including a polypeptide. The same ingredients as those disclose above with respect to immunogenic compositions of isolated nucleic acids may be utilized.

Methods of Enhancing or Promoting an Immune Response.

Methods of enhancing or promoting an immune response in an animal, including humans, to an antigen are also provided. Such methods can be practiced, for example, by identifying an animal in need of an immune response and administering said animal with any of the immunogenic compositions described above that is effective to enhance or facilitate an immune response to the second antigenic protein. In some embodiments, compositions including one or more isolated nucleic acids encoding the HBcAg antigen, or a fragment thereof, and a nucleic acid encoding a HDV protein (e.g., HDAg-L or HDAg-S) are administered to an animal in need thereof at the same time in the same mixture. In certain embodiments, compositions of HBcAg antigen, or a fragment thereof, and a HDV protein (e.g., HDAg-L or HDAg-S) are administered to the animal at the same time in the same mixture. Alternatively, the nucleic acid encoding the HBcAg and the nucleic acid encoding the HDV antigenic protein (e.g., HDAg-L or HDAg-S) are coadministered. Similarly, the HBcAg protein and the HDV polypeptide (e.g., HDAg-L or HDAg-S) can be coadministered. By coadministered, it is meant that the two or more nucleic acids, or two or more proteins, or one or more nucleic acid in combination with one or more protein are provided at the same time in the same mixture or within at least, equal to, or about any number in between 1, 5, 10, 15, 20, 30, 40, 50, or 60 minutes each separate administration. However, the present invention is not limited to any particular order of administration.

Accordingly, some methods include administering a composition comprising an isolated nucleic acid encoding HBcAg, or a fragment thereof, joined to an isolated nucleic acid encoding a HDV protein (e.g., HDAg-L or HDAg-S). Non-limiting examples of compositions that may be administered according to the methods disclosed herein include, but are not limited to nucleic acids encoding: stork HBcAg (nucleic acids of or encoding SEQ ID NOs:1-2) joined to HDAg (e.g., HDAg-L or HDAg-S); and heron HBcAg (nucleic acids of or encoding SEQ ID NOs:3-4) joined to HDAg (e.g., HDAg-L or HDAg-S).

Furthermore, compositions including nucleic acid sequences encoding HBcAg (such as nucleic acids of or encoding SEQ ID NOs:1-10), or a fragment thereof, and nucleic acid sequences encoding a HDV protein (e.g., HDAg-L or HDAg-S) in Trans, may be administered according to the methods disclosed herein. Non-limiting examples of compositions for administering according to the methods disclosed herein, include, but are not limited to nucleic acids encoding: stork HBcAg (nucleic acids of or encoding SEQ ID NOs:1-2) joined to HDAg (e.g., HDAg-L or HDAg-S); and heron HBcAg (nucleic acids of or encoding SEQ ID NOs:3-4) joined to HDAg (e.g., HDAg-L or HDAg-S).

In addition, compositions including HBcAg, or a fragment thereof, and a HDV protein (e.g., HDAg-L or HDAg-S), may be administered according to the methods disclosed herein. Non-limiting examples of the compositions for administering according to the methods disclosed herein, include, but are not limited to: stork HBcAg (SEQ ID NOs:1-2) joined to HDAg (e.g., HDAg-L or HDAg-S); and heron HBcAg (SEQ ID NOs:3-4) joined to HDAg (e.g., HDAg-L or HDAg-S).

The effective dose and method of administration of a particular formulation can vary based on the individual patient and the type and stage of the disease, as well as other factors known to those of skill in the art. Therapeutic efficacy and toxicity can be determined by standard pharmaceutical procedures in cell cultures or experimental animals, e.g., ED50 (the dose therapeutically effective in 50% of the population). The data obtained from cell culture assays and animal studies can be used to formulate a range of dosage for human use. The dosage lies preferably within a range of circulating concentrations that include the ED50 with no toxicity. The dosage varies within this range depending upon the type of adjuvant derivative and antigen, the dosage form employed, the sensitivity of the patient, and the route of administration.

In certain embodiments an adjuvant is included within the administered composition. For instance, a pharmacologic agent can be added to a composition described herein as needed to increase or aid its effect. In another example, an immunological agent that increases the antigenic response can be utilized with a device described herein. For instance, U.S. Pat. No. 6,680,059, published Jan. 20, 2004 (which is hereby incorporated in its entirety by reference) describes the use of vaccines containing ribavirin as an adjuvant to the vaccine. However, an adjuvant may refer to any material that has the ability to enhance or facilitate an immune response or to increase or aid the effect of a therapeutic agent. Non-limiting example of adjuvants include interleukin-2 (IL2), interleukin-12 (IL12), interleukin-15 (IL15), interleukin-21 (IL21), interleukin-28b (IL28b), galactosyl transferase, a toll-like receptor (TLR), ribavirin, alum, CpGs, and an oil. Also, as described above, in some embodiments, the composition includes an isolated nucleic acid, or constructs comprising said nucleic acids, encoding a protein that is an adjuvant, such as IL2, IL12, IL15, IL21, IL28b, galactosyl transferase, a TLR, and the like. In certain aspects, the isolated nucleic acid encoding the protein, which is an adjuvant may be in the same construct encoding HBcAg and/or the second antigenic protein. In some aspects, methods of administering the immunogenic composition comprise administering an adjuvant before administering the immunogenic composition.

In some embodiments, the method includes administering an immunogenic composition that comprises an isolated nucleic that encodes HBcAg (such as nucleic acids of or encoding SEQ ID NOs:1-10), or a fragment thereof, and separately administering an isolated nucleic acid that encodes a HDV protein (e.g., HDAg-L or HDAg-S). When the isolated nucleic acid encoding HBcAg and the isolated nucleic acid encoding the HDV protein (e.g., HDAg-L or HDAg-S) are administered separately, the isolated nucleic acid encoding HBcAg may, in some embodiments, may be administered before the isolated nucleic acid encoding second antigenic protein. Alternatively, the isolated nucleic acid encoding the HDV protein (e.g., HDAg-L or HDAg-S) may, in some embodiments, be administered before the isolated nucleic acid encoding HBcAg.

Other embodiments of the methods disclosed herein include administering a composition including both HBcAg and the HDV protein (e.g., HDAg-L or HDAg-S). In some embodiments, the method includes administering an immunogenic composition that includes an admixture of an isolated nucleic acid encoding HBcAg (such as nucleic acids of or encoding SEQ ID NOs:1-10) and an isolated nucleic acid encoding the HDV protein (e.g., HDAg-L or HDAg-S), preferably both being codon optimized for expression in humans. In certain embodiments, the method includes administering an immunogenic composition that includes an admixture of an isolated nucleic acid encoding the HBcAg and an isolated nucleic acid encoding the HDV protein (e.g., HDAg-L or HDAg-S).

Various routes of administration may be used for the methods described herein. In some embodiments, the immunogenic composition is administered parenterally (e.g., intramuscularly, intraperitoneally, subcutaneously, or intravenously to a mammal subject). In a preferred embodiment, the immunogenic compositions are administered intramuscularly, dermally, or subcutaneously. The methods may also include applying electrical stimulation, which can enhance the administration of the immunogenic compositions. As an example, electroporation may be included in the present methods disclosed herein. Electroporation includes applying electrical stimulation to improve the permeability of cells to the administered composition. Examples of electroporation techniques are disclosed in U.S. Pat. Nos. 6,610,044 and 5,273,525, the disclosures of both of these references are hereby incorporated by reference in their entireties.

The concentration of the nucleic acid or protein in the immunogenic composition to be administered can vary from about 0.1 ng/ml to about 50 mg/ml. In some aspects, the concentration of the immunogenic composition administered (e.g., a suitable dose of nucleic acid or protein for administration) is between about 10 ng/ml to 25 mg/ml. In still other aspects, the concentration is between 100 ng/ml to 10 mg/ml. In some aspects, the suitable dose of nucleic acid or protein for administration is greater than or equal to or less than about 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 μg/ml, 40 μg/ml, 41 μg/ml, 42 μg/ml, 43 μg/ml, 44 μg/ml, 45 μg/ml, 46 μg/ml, 47 μg/ml, 48 μg/ml, 49 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, 150 μg/ml, 200 μg/ml, 250 μg/ml, 300 μg/ml, 350 μg/ml, 400 μg/ml, 450 μg/ml, 500 μg/ml, 550 μg/ml, 600 μg/ml, 650 μg/ml, 700 μg/ml, 750 μg/ml, 800 μg/ml, 850 μg/ml, 900 μg/ml, 950 μg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3.0 mg/ml, 3.1 mg/ml, 3.2 mg/ml, 3.3 mg/ml, 3.4 mg/ml, 3.5 mg/ml, 3.6 mg/ml, 3.7 mg/ml, 3.8 mg/ml, 3.9 mg/ml, 4.0 mg/ml, 4.1 mg/ml, 4.2 mg/ml, 4.3 mg/ml, 4.4 mg/ml, 4.5 mg/ml, 4.6 mg/ml, 4.7 mg/ml, 4.8 mg/ml, 4.9 mg/ml, 5.0 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml, 6.0 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7.0 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8.0 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9.0 mg/ml, 9.1 mg/ml, 9.2 mg/ml, 9.3 mg/ml, 9.4 mg/ml, 9.5 mg/ml, 9.6 mg/ml, 9.7 mg/ml, 9.8 mg/ml, 9.9 mg/ml, 10.0 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, 40 mg/ml, 41 mg/ml, 42 mg/ml, 43 mg/ml, 44 mg/ml, 45 mg/ml, 46 mg/ml, 47 mg/ml, 48 mg/ml, 49 mg/ml, 50 mg/ml, or within a range defined by, and including, any two of these values.

The amount of nucleic acid or protein administered using the methods described herein can vary from about 1 ng to 10 g. In some aspects, the amount of nucleic acid or protein contained administered is less than greater than or equal to about 1 ng, 5 ng, 10 ng, 20 ng, 30 ng, 40 ng, 50 ng, 60 ng, 70 ng, 80 ng, 90 ng, 100 ng, 150 ng, 200 ng, 250 ng, 300 ng, 350 ng, 400 ng, 500 ng, 600 ng, 700 ng, 800 ng, 900 ng, 1 μg 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg 150 μg, 155 μg, 160 μg, 165 μg, 170 μg, 175 μg, 180 μg, 185 μg, 190 μg, 195 μg 200 μg, 205 μg, 210 μg, 215 μg, 220 μg, 225 μg, 230 μg, 235 μg, 240 μg, 245 μg 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg 350 μg, 355 μg, 360 μg, 365 μg, 370 μg, 375 μg, 380 μg, 385 μg, 390 μg, 395 μg 400 μg, 405 μg, 410 μg, 415 μg, 420 μg, 425 μg, 430 μg, 435 μg, 440 μg, 445 μg 450 μg, 455 μg, 460 μg, 465 μg, 470 μg, 475 μg, 480 μg, 485 μg, 490 μg, 495 μg 500 μg, 505 μg, 510 μg, 515 μg, 520 μg, 525 μg, 530 μg, 535 μg, 540 μg, 545 μg 550 μg, 555 μg, 560 μg, 565 μg, 570 μg, 575 μg, 580 μg, 585 μg, 590 μg, 595 μg 600 μg, 605 μg, 610 μg, 615 μg, 620 μg, 625 μg, 630 μg, 635 μg, 640 μg, 645 μg 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg 850 μg, 855 μg, 860 μg, 865 μg, 870 μg, 875 μg, 880 μg, 885 μg, 890 μg, 895 μg 900 μg, 905 μg, 910 μg, 915 μg, 920 μg, 925 μg, 930 μg, 935 μg, 940 μg, 945 μg 950 μg, 955 μg, 960 μg, 965 μg, 970 μg, 975 μg, 980 μg, 985 μg, 990 μg, 995 μg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, 10.0 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g, 2 g, 3 g, 4 g, 5 g, 6 g, 7 g, 8 g, 9 g, 10 g or within a range defined by, and including, any two of these values.

Materials and Methods.

In some embodiments, compositions are employed and methods performed according to the descriptions below. Other materials and methods are contemplated and consistent with the disclosure herein. Accordingly, the disclosure below should be read as enabling but not limiting to the claimed subject matter.

Materials and methods are drawn from Holmstrom et al., (2013) “A Synthetic Codon-Optimized Hepatitis C Polyfunctional CD8+ T Cell Responses in Virus Nonstructural 5A DNA Vaccine Primes Wild-Type and NS5A-Transgenic Mice” J Immunol 190:1113-1124, prepublished online Jan. 2, 2013, which is hereby incorporated by reference in its entirety for all content from pages 1113-1124.

The following sections are provided to illustrate various embodiments of the present invention. It is to be understood that the following discussion is not comprehensive or exhaustive of the many types of embodiments, which can be prepared in accordance with the present invention.

### Preferred Constructs and Evaluation for Immunogenicity

Preferred expression constructs comprising one or more of the nucleic acids described herein (see e.g., FIG. 1, and nucleic acids of or encoding SEQ ID NOs:85-148) are tested in animals to confirm that the introduction of self-cleavage sites into the fusion proteins encoded by the administered nucleic acids improve the immunogenicity (e.g., T cell and/or antibody response of the subject) of the immunogenic compositions. The immunogenicity of several constructs are evaluated after introducing the constructs into animals using the IVIN injector with electroporation (see PCT/IB2012/001321, WO 2012/172424 A1), which was published in English on Dec. 20, 2012 and designated the United States, hereby expressly incorporated by reference in its entirety). In a first set of experiments, the following constructs are evaluated:

(1) expression constructs comprising a nucleic acid encoding a wild-type HDAg-L or HDAg-S sequence or both;

(2) expression constructs comprising a nucleic acid encoding a HDAg-L or HDAg-S sequence or both, wherein said nucleic acid is codon optimized for expression in humans;

(3) expression constructs comprising a nucleic acid encoding a HDAg-L or HDAg-S sequence or both, wherein said nucleic acid is codon optimized for expression in humans and wherein said nucleic acid additionally encodes a self cleavage sequence, which may also be codon optimized for expression in humans (e.g., P2A, E2A, F2A, or T2A with or without GSG modification) within said HDAg-L or said HDAg-S sequence or both or at the N or C terminus of said HDAg-L or said HDAg-S sequence or both;

(4) expression constructs comprising a nucleic acid encoding a HDAg-L or HDAg-S sequence or both, wherein said nucleic acid is codon optimized for expression in humans and wherein said nucleic acid, optionally encodes a self cleavage sequence, which may also be codon optimized for expression in humans (e.g., P2A, E2A, F2A, or T2A with or without GSG modification) within said HDAg-L or said HDAg-S sequence or both or at the N or C terminus of said HDAg-L or said HDAg-S sequence or both, and, wherein said expression construct additionally comprises a nucleic acid sequence encoding an HBcAg, which may also be codon optimized for expression in humans (e.g., a codon optimized stork or heron HBcAg) and said nucleic acid encoding said HBcAg is joined to said nucleic acid encoding HDAg-L or HDAg-S sequence either directly, through a linker, or vis a vis a self cleavage site (e.g., P2A, E2A, F2A, or T2A with or without GSG modification) that is joined at the N or C terminus of said HDAg-L or said HDAg-S sequence (e.g., a P2A site existing between said HDAg-L or HDAg-S sequence and said HBcAg sequence);

(5) expression constructs comprising a nucleic acid encoding a HDAg-L or HDAg-S sequence or both, wherein said nucleic acid is codon optimized for expression in humans and wherein said nucleic acid, optionally encodes a self cleavage sequence, which may also be codon optimized for expression in humans (e.g., P2A, E2A, F2A, or T2A with or without GSG modification) within said HDAg-L or said HDAg-S sequence or both or at the N or C terminus of said HDAg-L or said HDAg-S sequence or both, and, wherein said expression construct additionally comprises a nucleic acid sequence encoding an HBcAg, which may also be codon optimized for expression in humans (e.g., a codon optimized stork or heron HBcAg) and said nucleic acid encoding said HBcAg is joined to said nucleic acid encoding HDAg-L or HDAg-S sequence either directly, through a linker, or vis a vis a self cleavage site (e.g., P2A, E2A, F2A, or T2A with or without GSG modification) that is joined at the N or C terminus of said HDAg-L or said HDAg-S sequence (e.g., a P2A site existing between said HDAg-L or HDAg-S sequence and said HBcAg sequence) and further, wherein said expression construct comprises a nucleic acid encoding one or more self-cleavage sites (e.g., P2A, E2A, F2A, or T2A with or without GSG modification) within said HBcAg sequence; and

(6) expression constructs comprising a nucleic acid encoding one or more of the fusion polypeptides illustrated in FIG. 1.

Assays are then performed to determine the relative impact of having self-cleavage polypeptide sequences in the constructs encoding the HBcAg and/or HDV polypeptides. Methods are performed largely as described in Antony Chen, Gustaf Ahlen, Erwin D. Brenndörfer, Anette Brass, Fredrik Holmstrom, Margaret Chen, Jonas Soderholm, David R. Milich, Lars Frelin and Matti Sallberg (2011) Heterologous T Cells Can Help Restore Function in Dysfunctional Hepatitis C Virus Nonstructural 3/4A-Specific T Cells during Therapeutic Vaccination. J Immunol 186:5107-5118, the contents of which are hereby incorporated by reference in their entirety as to the entire disclosure of pages 5107 through 5118 inclusive. In sum, the immunogenicity of the constructs tested are evaluated after introducing the constructs into animals using the IVIN injector with electroporation (see PCT/IB2012/001321 (WO 2012/172424 A1, published Dec. 20, 2012), hereby expressly incorporated by reference in its entirety. After administration of the various constructs to the animals, with or without additional boosts, the immunogenicity of the constructs are evaluated (e.g., T helper and CTL-specific immune responses, cytokine responses, and/or antibody responses are evaluated and the efficacy of the various constructs tested are compared). It will be determined that the construct comprising the codon-optimized sequence encoding HDAg-L or HDAg-S sequence or both will be more immunogenic (e.g., stronger T helper and CTL-specific immune responses, cytokine responses, and/or antibody responses) than the construct encoding wild-type HDAg-L or HDAg-S sequence or both. It will also be determined that the construct encoding a fusion of HBcAg (e.g., a nucleic acid encoding an avian HBcAg that has been codon optimized for expression in humans) with HDAg-L or HDAg-S sequence or both will be more immunogenic (e.g., stronger T helper and CTL-specific immune responses, cytokine responses, and/or antibody responses) than the construct encoding a HDAg-L or HDAg-S sequence or both lacking HBcAg. It will also be determined that the construct encoding a fusion of HBcAg (e.g., a nucleic acid encoding an avian HBcAg that has been codon optimized for expression in humans) with HDAg-L or HDAg-S sequence or both, wherein said HBcAg is joined to a self-cleavage site (e.g., a P2A site) at either the N or C terminus will be more immunogenic (e.g., stronger T helper and CTL-specific immune responses, cytokine responses, and/or antibody responses) than the construct encoding a HDAg-L or HDAg-S sequence or both joined to HBcAg but lacking a self cleavage site. It will also be demonstrated that constructs having a nucleic acid encoding a fusion of HBcAg (e.g., a nucleic acid encoding an avian HBcAg that has been codon optimized for expression in humans) with HDAg-L or HDAg-S sequence or both, wherein a self-cleavage site (e.g., a P2A site) exists between said HBcAg and said HDAg-L or HDAg-S sequence or both will be more immunogenic (e.g., stronger T helper and CTL-specific immune responses, cytokine responses, and/or antibody responses) than the construct encoding a HDAg-L or HDAg-S sequence or both joined to HBcAg but lacking a self cleavage site. It will also be demonstrated that the construct encoding a fusion of HBcAg (e.g., a nucleic acid encoding an avian HBcAg that has been codon optimized for expression in humans) with HDAg-L or HDAg-S sequence or both, wherein said HBcAg comprises a plurality of self-cleavage sites (e.g., a P2A site) at the N and/or C terminus and/or internally positioned will be more immunogenic (e.g., stronger T helper and CTL-specific immune responses, cytokine responses, and/or antibody responses) than the construct encoding a HDAg-L or HDAg-S sequence or both joined to HBcAg but lacking a self cleavage site, as well as, constructs having only a single self cleavage site.

### Administration Regimen

Truncated Therapeutic administration of a therapy for HDV infection is performed in patients with HDV infection. Some patients who receive a booster dose start treatment within 1-2 months after the booster dose. Treatment begins after a mean interval of 15 months (range 1-30) from last administration.

Patients are preferably HDV treatment naïve. Patients receive administrations of an HDV-containing immunogenic composition (e.g., one or more of the constructs depicted in FIG. 1 in the deltoid muscles (e.g., four monthly administrations with 167 μg, 500 μg, or 1.500 μg codon-optimized HDV HDAg-P2A-HBcAg immunogen formulations delivered by in vivo electroporation (EP) in some approaches). Enrollment may be done with two weeks interval between patients for safety reasons. It is expected that the administration will significantly improve IFNγ producing responses to HDAg during the first six weeks of therapy. Patients are expected to experience 0.6 log 10-2.4 log 10 reduction in serum HDV RNA and some are expected to be effectively treated (e.g., HDV viral titer is reduced) or cured (e.g., HDV viral titer is reduced to undetectable amounts by a clinical assay).

Patients are administered the therapy and in one minute or less electroporation is performed, for example as described in PCT Publication No. WO 2012/172424 A1, published Dec. 20, 2012, which is hereby incorporated by reference in its entirety not only as it relates to electroporation but for all content disclosed therein.

By some approaches, a volume of 0.5 mL 0.9% sodium chloride containing the DNA is injected in the deltoid muscle (alternating left and right) using an IVIN needle at a depth of 1.2 cm. The injection site is marked prior to injection with a surgical pen and then sterilized by swiping with an alcohol pad Immediately after the injection or along with the injection an IVIN-based electroporator is used at the site of injection and electroporation is administered, as described, for example, in PCT Publication No. WO 2012/172424 A1, published Dec. 20, 2012, incorporated by reference in its entirety here and above. The administration is expected to be safe and well tolerated by recipients.

Patients will demonstrate an increase in relative antibody levels detected by a paired comparison of the samples obtained at week 0 and 2, an effect, which is most pronounced in the two lowest dose groups. Some patients will demonstrate de novo T cell activation. The presence of HDV specific T cell responses before, during and after the therapeutic administration is determined as the number of IFNγ-producing T cells, or spot forming cells (SFCs) by ELISpot, and the level of proliferation as determined by the level of [3]H-thymidine incorporation. In the ELISpot assay, only the responses to nine peptide pools spanning the whole HDAg region are used for the statistical comparison to avoid repeated use of the same epitope and to overcome HLA-restriction.

The number of the IFNγ-producing spots are expected to increase after the two first vaccinations when comparing the number of SFCs at week 0, and the same at weeks 2 and 6. Proliferative T cell responses to HDAg are detected in a substantial number of subjects prior to or after vaccination. de novo ELISpot responses are observed in a fraction of all groups observed. In some patients the activation, or reactivation, of HDV HDAg IFNγ-producing T cells coincides with the suppression of the HDV RNA levels in blood.

A rapid viral response, and complete early viral response and sustained viral response will be seen in a substantial number of patients.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

Aspects of the Invention May Include One or More of the Following, Alone or in Combination

```
SEQ ID NO: 1
coSHBcAg - stork
GTGAACGCCAGCAGAGCCCTGGCCAACGTGTACGACCTGCCCGACGACTTCTTCCCCCAGAT
CGACGACCTGGTGAGAGACGCCAAGGACGCCCTGGAGCCCTACTGGAAGGCCGAGACCATCA
AGAAGCACGTGCTGATCGCCACCCACTTCGTGGACTTGATCGAGGACTTCTGGCAGACCACC
CAGGGCATGAGCCAGATCGCCGACGCCCTGAGAGCCGTGATCCCCCCCACCACCACCCCCGT
GCCCGACGGCTACCTGATCAGCCACAACGAGGCCCAGGAGCTCCCCCTGAACGACCTGTTCG
TGCTGCAGGAGGAGAGAATCGTGAACTTCCAGCCCGACTACCCCATCACCGCCAGAATTCAC
ACCCACCTGAGAGTGTACACCAAGCTGAACGAGCAGGCCCTGGACAAGGCCAGAAGACTGCT
GTGGTGGCACTACAACTGCCTGCTGTGGGGCGAGAGCAACGTGACCAACTACATCAGCAGAC
TGAGAACCTGGCTGAGCACCCCCGAGAAGTACAGAGGCAAGGACGCCCCCACCATCGAGGCC
ATCACCAGACCCATCCAGGTGGCCCAGGGCAGCAGAAACCAGACCAAGGGCGTGAGAAAGCC
CAGAGGCCTGGAGCCCAGAAGAAGAAAGGTGAAGACCACCGTGGTGTACGGCAGAAGAAGAA
GCAAGAGCAGAGGCAGAAGAAGCAGCCCCAGCCAGAGAGCCGGCAGCCCCATCCCCAGAAAC
AGAGAGAACCAGAGCAGAAGCAGCAGCCCCAGAGAG
SEQ ID NO: 2
SHBcAg polypeptide - stork
ValAsnAlaSerArgAlaLeuAlaAsnValTyrAspLeuProAspAspPhePhe
ProGlnIleAspAspLeuValArgAspAlaLysAspAlaLeuGluProTyrTrpLysAla
GluThrIleLysLysHisValLeuIleAlaThrHisPheValAspLeuIleGluAspPhe
TrpGlnThrThrGlnGlyMetSerGlnIleAlaAspAlaLeuArgAlaValIleProPro
ThrThrThrProValProAspGlyTyrLeuIleSerHisAsnGluAlaGlnGluLeuPro
LeuAsnAspLeuPheValLeuGlnGluGluArgIleValAsnPheGlnProAspTyrPro
IleThrAlaArgIleHisThrHisLeuArgValTyrThrLysLeuAsnGluGlnAlaLeu
AspLysAlaArgArgLeuLeuTrpTrpHisTyrAsnCysLeuLeuTrpGlyGluSerAsn
ValThrAsnTyrIleSerArgLeuArgThrTrpLeuSerThrProGluLysTyrArgGly
LysAspAlaProThrIleGluAlaIleThrArgProIleGlnValAlaGlnGlySerArg
AsnGlnThrLysGlyValArgLysProArgGlyLeuGluProArgArgArgLysValLys
ThrThrValValTyrGlyArgArgArgSerLysSerArgGlyArgArgSerSerProSer
GlnArgAlaGlySerProIleProArgAsnArgGluAsnGlnSerArgSerSerSerPro
ArgGlu
SEQ ID NO: 3
coHHBcAg - heron
GTGAACGCCAGCAGAGCCCTGGCCAACGTGTACGACCTGCCCGACGACTTCTTCCCCCAGAT
CGACGACCTGGTGAGAGACGCCAAGGACGCCCTGGAGCCCTACTGGAAGGCCGAGACCATCA
AGAAGCACGTGCTGATCGCCACCCACTTCGTGGACTTGATCGAGGACTTCTGGCAGACCACC
CAGGGCATGAGCCAGATCGCCGACGCCCTGAGAGCCGTGATCCCCCCCACCACCGTGCCCGT
GCCCGAGGGCTTCCTGATCACCCACAGCGAGGCCGAGGAGCTCCCCCTGAACGACCTGTTCA
GTCTGCAGGAGGAGAGAATCGTGAACTTCCAGCCCGACTACCCCATCACCGCCAGAATTCAC
ACCCACCTGAGAGTGTACACCAAGCTGAACGAGCAGGCCCTGGACAAGGCCAGAAGACTGCT
GTGGTGGCACTACAACTGCCTGCTGTGGGGCGAGGCCACCGTGACCAACTACATCAGCAGAC
TGAGAACCTGGCTGAGCACCCCCGAGAAGTACAGAGGCAAGGACGCCCCCACCATCGAGGCC
ATCACCAGACCCATCCAGGTGGCCCAGGGCGGCAGAAACCAGACCAAGGGCACCAGAAAGCC
CAGAGGCCTGGAGCCCAGAAGAAGAAAGGTGAAGACCACCGTGGTGTACGGCAGAAGAAGAA
GCAAGAGCAGAGGCAGAAGAAGCAGCCCCAGCCAGAGAGCCGGCAGCCCCCTGCCCAGAAAC
AGAGGCAACCAGACCAGAAGCCCCAGCCCCAGAGAG
SEQ ID NO: 4
HHBcAg - heron 262 residues
ValAsnAlaSerArgAlaLeuAlaAsnValTyrAspLeuProAspAspPhePhe
ProGlnIleAspAspLeuValArgAspAlaLysAspAlaLeuGluProTyrTrpLysAla
GluThrIleLysLysHisValLeuIleAlaThrHisPheValAspLeuIleGluAspPhe
TrpGlnThrThrGlnGlyMetSerGlnIleAlaAspAlaLeuArgAlaValIleProPro
ThrThrValProValProGluGlyPheLeuIleThrHisSerGluAlaGluGluLeuPro
LeuAsnAspLeuPheSerLeuGlnGluGluArgIleValAsnPheGlnProAspTyrPro
IleThrAlaArgIleHisThrHisLeuArgValTyrThrLysLeuAsnGluGlnAlaLeu
AspLysAlaArgArgLeuLeuTrpTrpHisTyrAsnCysLeuLeuTrpGlyGluAlaThr
ValThrAsnTyrIleSerArgLeuArgThrTrpLeuSerThrProGluLysTyrArgGly
LysAspAlaProThrIleGluAlaIleThrArgProIleGlnValAlaGlnGlyGlyArg
AsnGlnThrLysGlyThrArgLysProArgGlyLeuGluProArgArgArgLysValLys
ThrThrValValTyrGlyArgArgArgSerLysSerArgGlyArgArgSerSerProSer
GlnArgAlaGlySerProLeuProArgAsnArgGlyAsnGlnThrArgSerProSerPro
ArgGlu
SEQ ID NO: 5
coHBcAg subtype ayw
ATCGACCCCTACAAGGAGTTCGGCGCCACCGTGGAGCTGCTGAGCTTCCTGCCCAGCGACTT
CTTCCCCAGCGTGAGAGACCTGCTGGACACCGCCAGCGCCCTGTACAGAGAGGCCCTGGAGA
GCCCCGAGCACTGCAGCCCCCACCACACCGCCCTGAGACAGGCCATCCTGTGCTGGGGCGAG
CTGATGACCCTGGCCACCTGGGTGGGCGTGAACCTGGAGGACCCCGCCAGCAGAGACCTGGT
GGTGAGCTACGTGAACACCAACATGGGCCTGAAGTTCAGACAGCTGCTGTGGTTCCACATCA
GCTGCCTGACCTTCGGCAGAGAGACCGTGATCGAGTACCTGGTGAGCTTCGGCGTGTGGATC
AGAACCCCCCCCGCCTACAGACCCCCCAACGCCCCCATCCTGAGCACCCTGCCCGAGACCAC
CGTGGTGAGAAGAAGAGGCAGAAGCCCCAGAAGAAGAACCCCCAGCCCCAGAAGAAGAAGAA
GCCAGAGCCCCAGAAGAAGAAGAAGCCAGAGCAGAGAGAGCCAGTGC
SEQ ID NO: 6
HBcAg subtype ayw protein - 183
IleAspProTyrLysGluPheGlyAlaThrValGluLeuLeuSerPheLeuPro
SerAspPhePheProSerValArgAspLeuLeuAspThrAlaSerAlaLeuTyrArgGlu
AlaLeuGluSerProGluHisCysSerProHisHisThrAlaLeuArgGlnAlaIleLeu
CysTrpGlyGluLeuMetThrLeuAlaThrTrpValGlyValAsnLeuGluAspProAla
SerArgAspLeuValValSerTyrValAsnThrAsnMetGlyLeuLysPheArgGlnLeu
LeuTrpPheHisIleSerCysLeuThrPheGlyArgGluThrValIleGluTyrLeuVal
SerPheGlyValTrpIleArgThrProProAlaTyrArgProProAsnAlaProIleLeu
SerThrLeuProGluThrThrValValArgArgArgGlyArgSerProArgArgArgThr
ProSerProArgArgArgArgSerGlnSerProArgArgArgArgSerGlnSerArgGlu
SerGlnCys
SEQ ID NO: 7
coHBeAg subtype ayw
CTGTTCCACCTGTGCCTGATCATCAGCTGCAGCTGCCCCACCGTGCAGGCCAGCAAGCTGTG
CCTGGGCTGGCTGTGGGGCATGGACATCGACCCCTACAAGGAGTTCGGCGCCACCGCCGAGC
TGCTGAGCTTCCTGCCCAGCGACTTCTTCCCCAGCGTGAGAGACCTGCTGGACACCGCCAGC
GCCCTGTACAGAGAGGCCCTGGAGAGCCCCGAGCACTGCAGCCCCCACCACACCGCCCTGAG
ACAGGCCATCCTGTGCTGGGGCGAGCTGATGACCCTGGCCACCTGGGTGGGCGTGAACCTGG
AGGACCCCGCCAGCAGAGACCTGGTGGTGAGCTACGTGAACACCAACATGGGCCTGAAGTTC
AGACAGCTGCTGTGGTTCCACATCAGCTGCCTGACCTTCGGCAGAGAGACCGTGATCGAGTA
CCTGGTGAGCTTCGGCGTGTGGATCAGAACCCCCCCCGCCTACAGACCCCCCAACGCCCCCA
TCCTGAGCACCCTGCCCGAGACCACCGTGGTGAGAAGAAGAGGCAGAAGCCCCAGAAGAAGA
ACCCCCAGCCCCAGAAGAAGAAGAAGCCAGAGCCCCAGAAGAAGAAGAAGCCAGAGCAGAGA
GAGCCAGTGC
SEQ ID NO: 8
HBeAg subtype ayw protein - 212
LeuPheHisLeuCysLeuIleIleSerCysSerCysProThrValGlnAlaSer
LysLeuCysLeuGlyTrpLeuTrpGlyMetAspIleAspProTyrLysGluPheGlyAla
ThrAlaGluLeuLeuSerPheLeuProSerAspPhePheProSerValArgAspLeuLeu
AspThrAlaSerAlaLeuTyrArgGluAlaLeuGluSerProGluHisCysSerProHis
HisThrAlaLeuArgGlnAlaIleLeuCysTrpGlyGluLeuMetThrLeuAlaThrTrp
ValGlyValAsnLeuGluAspProAlaSerArgAspLeuValValSerTyrValAsnThr
AsnMetGlyLeuLysPheArgGlnLeuLeuTrpPheHisIleSerCysLeuThrPheGly
ArgGluThrValIleGluTyrLeuValSerPheGlyValTrpIleArgThrProProAla
TyrArgProProAsnAlaProIleLeuSerThrLeuProGluThrThrValValArgArg
ArgGlyArgSerProArgArgArgThrProSerProArgArgArgArgSerGlnSerPro
ArgArgArgArgSerGlnSerArgGluSerGlnCys
SEQ ID NO: 9
coHBcAg ayw (no internal start)
CTGTTCCACCTGTGCCTGATCATCAGCTGCAGCTGCCCCACCGTGCAGGCCAGCAAGCTGTG
CCTGGGCTGGCTGTGGGGCCTGGACATCGACCCCTACAAGGAGTTCGGCGCCACCGCCGAGC
TGCTGAGCTTCCTGCCCAGCGACTTCTTCCCCAGCGTGAGAGACCTGCTGGACACCGCCAGC
GCCCTGTACAGAGAGGCCCTGGAGAGCCCCGAGCACTGCAGCCCCCACCACACCGCCCTGAG
ACAGGCCATCCTGTGCTGGGGCGAGCTGATGACCCTGGCCACCTGGGTGGGCGTGAACCTGG
AGGACCCCGCCAGCAGAGACCTGGTGGTGAGCTACGTGAACACCAACATGGGCCTGAAGTTC
AGACAGCTGCTGTGGTTCCACATCAGCTGCCTGACCTTCGGCAGAGAGACCGTGATCGAGTA
CCTGGTGAGCTTCGGCGTGTGGATCAGAACCCCCCCCGCCTACAGACCCCCCAACGCCCCCA
TCCTGAGCACCCTGCCCGAGACCACCGTGGTGAGAAGAAGAGGCAGAAGCCCCAGAAGAAGA
ACCCCCAGCCCCAGAAGAAGAAGAAGCCAGAGCCCCAGAAGAAGAAGAAGCCAGAGCAGAGA
GAGCCAGTGC
SEQ ID NO: 10
HBcAg ayw (no internal start) protein
LeuPheHisLeuCysLeuIleIleSerCysSerCysProThrValGlnAlaSer
LysLeuCysLeuGlyTrpLeuTrpGlyLeuAspIleAspProTyrLysGluPheGlyAla
ThrAlaGluLeuLeuSerPheLeuProSerAspPhePheProSerValArgAspLeuLeu
AspThrAlaSerAlaLeuTyrArgGluAlaLeuGluSerProGluHisCysSerProHis
HisThrAlaLeuArgGlnAlaIleLeuCysTrpGlyGluLeuMetThrLeuAlaThrTrp
ValGlyValAsnLeuGluAspProAlaSerArgAspLeuValValSerTyrValAsnThr
AsnMetGlyLeuLysPheArgGlnLeuLeuTrpPheHisIleSerCysLeuThrPheGly
ArgGluThrValIleGluTyrLeuValSerPheGlyValTrpIleArgThrProProAla
TyrArgProProAsnAlaProIleLeuSerThrLeuProGluThrThrValValArgArg
ArgGlyArgSerProArgArgArgThrProSerProArgArgArgArgSerGlnSerPro
ArgArgArgArgSerGlnSerArgGluSerGlnCys
SEQ ID NO: 11
P2A
GCTACTAACTTCAGCCTGCTGAAGCAGGCTGGAGACGTGGAGGAGAACCCTGGACCT
SEQ ID NO: 12
P2A polypeptide sequence
AlaThrAsnPheSerLeuLeuLysGlnAlaGlyAspValGluGluAsnPro
GlyPro
SEQ ID NO: 13
E2A
CAGTGTACTAATTATGCTCTCTTGAAATTGGCTGGAGATGTTGAGAGCAACCCTGGACCT
SEQ ID NO: 14
E2A polypeptide sequence
GlnCysThrAsnTyrAlaLeuLeuLysLeuAlaGlyAspValGluSerAsnProGlyPro
SEQ ID NO: 15
F2A
GTGAAACAGACTTTGAATTTTGACCTTCTCAAGTTGGCGGGAGACGTGGAGTCCAACCCTGG
ACCT
SEQ ID NO: 16
F2A polypeptide sequence
ValLysGlnThrLeuAsnPheAspLeuLeuLysLeuAlaGlyAspValGluSerAsnPro
GlyPro
SEQ ID NO: 17
T2A
GAGGGCAGAGGAAGTCTGCTAACATGCGGTGACGTCGAGGAGAATCCTGGACCT
SEQ ID NO: 18
T2A polypeptide sequence
GluGlyArgGlySerLeuLeuThrCysGlyAspValGluGluAsnProGlyPro
SEQ ID NO: 19
GSG Sequence
GGAAGCGGA
SEQ ID NO: 20
GSG polypeptide sequence
GlySerGly
SEQ ID NO: 21
GSG-P2A
GGAAGCGGAGCTACTAACTTCAGCCTGCTGAAGCAGGCTGGAGACGTGGAGGAGAACCCTGG
ACCT
SEQ ID NO: 22
GSG-P2A polypeptide sequence
GlySerGlyAlaThrAsnPheSerLeuLeuLysGlnAlaGlyAspValGluGluAsnPro
GlyPro
SEQ ID NO: 23
P2A-coSHBcAg
GCTACTAACTTCAGCCTGCTGAAGCAGGCTGGAGACGTGGAGGAGAACCCTGGACCTATGGA
CGTGAACGCCAGCAGAGCCCTGGCCAACGTGTACGACCTGCCCGACGACTTCTTCCCCCAGA
TCGACGACCTGGTGAGAGACGCCAAGGACGCCCTGGAGCCCTACTGGAAGGCCGAGACCATC
AAGAAGCACGTGCTGATCGCCACCCACTTCGTGGACTTGATCGAGGACTTCTGGCAGACCAC
CCAGGGCATGAGCCAGATCGCCGACGCCCTGAGAGCCGTGATCCCCCCCACCACCACCCCCG
TGCCCGACGGCTACCTGATCAGCCACAACGAGGCCCAGGAGCTCCCCCTGAACGACCTGTTC
GTGCTGCAGGAGGAGAGAATCGTGAACTTCCAGCCCGACTACCCCATCACCGCCAGAATTCA
CACCCACCTGAGAGTGTACACCAAGCTGAACGAGCAGGCCCTGGACAAGGCCAGAAGACTGC
TGTGGTGGCACTACAACTGCCTGCTGTGGGGCGAGAGCAACGTGACCAACTACATCAGCAGA
CTGAGAACCTGGCTGAGCACCCCCGAGAAGTACAGAGGCAAGGACGCCCCCACCATCGAGGC
CATCACCAGACCCATCCAGGTGGCCCAGGGCAGCAGAAACCAGACCAAGGGCGTGAGAAAGC
CCAGAGGCCTGGAGCCCAGAAGAAGAAAGGTGAAGACCACCGTGGTGTACGGCAGAAGAAGA
AGCAAGAGCAGAGGCAGAAGAAGCAGCCCCAGCCAGAGAGCCGGCAGCCCCATCCCCAGAAA
CAGAGAGAACCAGAGCAGAAGCAGCAGCCCCAGAGAG
SEQ ID NO: 24
P2A-coSHBcAg polypeptide
AlaThrAsnPheSerLeuLeuLysGlnAlaGlyAspValGluGluAsnProGlyProMet
AspValAsnAlaSerArgAlaLeuAlaAsnValTyrAspLeuProAspAspPhePhePro
GlnIleAspAspLeuValArgAspAlaLysAspAlaLeuGluProTyrTrpLysAlaGlu
ThrIleLysLysHisValLeuIleAlaThrHisPheValAspLeuIleGluAspPheTrp
GlnThrThrGlnGlyMetSerGlnIleAlaAspAlaLeuArgAlaValIleProProThr
ThrThrProValProAspGlyTyrLeuIleSerHisAsnGluAlaGlnGluLeuProLeu
AsnAspLeuPheValLeuGlnGluGluArgIleValAsnPheGlnProAspTyrProIle
ThrAlaArgIleHisThrHisLeuArgValTyrThrLysLeuAsnGluGlnAlaLeuAsp
LysAlaArgArgLeuLeuTrpTrpHisTyrAsnCysLeuLeuTrpGlyGluSerAsnVal
ThrAsnTyrIleSerArgLeuArgThrTrpLeuSerThrProGluLysTyrArgGlyLys
AspAlaProThrIleGluAlaIleThrArgProIleGlnValAlaGlnGlySerArgAsn
GlnThrLysGlyValArgLysProArgGlyLeuGluProArgArgArgLysValLysThr
ThrValValTyrGlyArgArgArgSerLysSerArgGlyArgArgSerSerProSerGln
ArgAlaGlySerProIleProArgAsnArgGluAsnGlnSerArgSerSerSerProArg
Glu
SEQ ID NO: 25
GSG-P2A-coSHBcAg
GGAAGCGGA
GCTACTAACTTCAGCCTGCTGAAGCAGGCTGGAGACGTGGAGGAGAACCCTGGACCTATGGA
CGTGAACGCCAGCAGAGCCCTGGCCAACGTGTACGACCTGCCCGACGACTTCTTCCCCCAGA
TCGACGACCTGGTGAGAGACGCCAAGGACGCCCTGGAGCCCTACTGGAAGGCCGAGACCATC
AAGAAGCACGTGCTGATCGCCACCCACTTCGTGGACTTGATCGAGGACTTCTGGCAGACCAC
CCAGGGCATGAGCCAGATCGCCGACGCCCTGAGAGCCGTGATCCCCCCCACCACCACCCCCG
TGCCCGACGGCTACCTGATCAGCCACAACGAGGCCCAGGAGCTCCCCCTGAACGACCTGTTC
GTGCTGCAGGAGGAGAGAATCGTGAACTTCCAGCCCGACTACCCCATCACCGCCAGAATTCA
CACCCACCTGAGAGTGTACACCAAGCTGAACGAGCAGGCCCTGGACAAGGCCAGAAGACTGC
TGTGGTGGCACTACAACTGCCTGCTGTGGGGCGAGAGCAACGTGACCAACTACATCAGCAGA
CTGAGAACCTGGCTGAGCACCCCCGAGAAGTACAGAGGCAAGGACGCCCCCACCATCGAGGC
CATCACCAGACCCATCCAGGTGGCCCAGGGCAGCAGAAACCAGACCAAGGGCGTGAGAAAGC
CCAGAGGCCTGGAGCCCAGAAGAAGAAAGGTGAAGACCACCGTGGTGTACGGCAGAAGAAGA
AGCAAGAGCAGAGGCAGAAGAAGCAGCCCCAGCCAGAGAGCCGGCAGCCCCATCCCCAGAAA
CAGAGAGAACCAGAGCAGAAGCAGCAGCCCCAGAGAG
SEQ ID NO: 26
GSG-P2A-e oSHBeAg polypeptide
GlySerGly
AlaThrAsnPheSerLeuLeuLysGlnAlaGlyAspValGluGluAsnProGlyProMet
AspValAsnAlaSerArgAlaLeuAlaAsnValTyrAspLeuProAspAspPhePhePro
GlnIleAspAspLeuValArgAspAlaLysAspAlaLeuGluProTyrTrpLysAlaGlu
ThrIleLysLysHisValLeuIleAlaThrHisPheValAspLeuIleGluAspPheTrp
GlnThrThrGlnGlyMetSerGlnIleAlaAspAlaLeuArgAlaValIleProProThr
ThrThrProValProAspGlyTyrLeuIleSerHisAsnGluAlaGlnGluLeuProLeu
AsnAspLeuPheValLeuGlnGluGluArgIleValAsnPheGlnProAspTyrProIle
ThrAlaArgIleHisThrHisLeuArgValTyrThrLysLeuAsnGluGlnAlaLeuAsp
LysAlaArgArgLeuLeuTrpTrpHisTyrAsnCysLeuLeuTrpGlyGluSerAsnVal
ThrAsnTyrIleSerArgLeuArgThrTrpLeuSerThrProGluLysTyrArgGlyLys
AspAlaProThrIleGluAlaIleThrArgProIleGlnValAlaGlnGlySerArgAsn
GlnThrLysGlyValArgLysProArgGlyLeuGluProArgArgArgLysValLysThr
ThrValValTyrGlyArgArgArgSerLysSerArgGlyArgArgSerSerProSerGln
ArgAlaGlySerProIleProArgAsnArgGluAsnGlnSerArgSerSerSerProArg
Glu
SEQ ID NO: 27
P2A-coSHBcAg multiple sites
GCTACTAACTTCAGCCTGCTGAAGCAGGCTGGAGACGTGGAGGAGAACCCTGGACCTATGGA
CGTGAACGCCAGCAGAGCCCTGGCCAACGTGTACGACCTGCCCGACGACTTCTTCCCCCAGA
TCGACGACCTGGTGAGAGACGCCAAGGACGCCCTGGAGCCCTACTGGAAGGCCGAGACCATC
AAGAAGCACGTGCTGATCGCCACCCACTTCGTGGACTTGATCGAGGACTTCTGGCAGACCAC
CCAGGGCGGAAGCGGAGCTACTAACTTCAGCCTGCTGAAGCAGGCTGGAGACGTGGAGGAGA
ACCCTGGACCTATGAGCCAGATCGCCGACGCCCTGAGAGCCGTGATCCCCCCCACCACCACC
CCCGTGCCCGACGGCTACCTGATCAGCCACAACGAGGCCCAGGAGCTCCCCCTGAACGACCT
GTTCGTGCTGCAGGAGGAGAGAATCGTGAACTTCCAGCCCGACTACCCCATCACCGCCAGAA
TTCACACCCACCTGAGAGTGTACGGAAGCGGAGCTACTAACTTCAGCCTGCTGAAGCAGGCT
GGAGACGTGGAGGAGAACCCTGGACCTACCAAGCTGAACGAGCAGGCCCTGGACAAGGCCAG
AAGACTGCTGTGGTGGCACTACAACTGCCTGCTGTGGGGCGAGAGCAACGTGACCAACTACA
TCAGCAGACTGAGAACCTGGCTGAGCACCCCCGAGAAGTACAGAGGCAAGGACGCCCCCACC
ATCGAGGCCATCACCAGACCCATCCAGGTGGCCCAGGGCGGAAGCGGAGCTACTAACTTCAG
CCTGCTGAAGCAGGCTGGAGACGTGGAGGAGAACCCTGGACCTAGCAGAAACCAGACCAAGG
GCGTGAGAAAGCCCAGAGGCCTGGAGCCCAGAAGAAGAAAGGTGAAGACCACCGTGGTGTAC
GGCAGAAGAAGAAGCAAGAGCAGAGGCAGAAGAAGCAGCCCCAGCCAGAGAGCCGGCAGCCC
CATCCCCAGAAACAGAGAGAACCAGAGCAGAAGCAGCAGCCCCAGAGAG
SEQ ID NO: 28
P2A-coSHBcAg multiple sites- polypeptide
AlaThrAsnPheSerLeuLeuLysGlnAlaGlyAspValGluGluAsnProGlyProMet
AspValAsnAlaSerArgAlaLeuAlaAsnValTyrAspLeuProAspAspPhePhePro
GlnIleAspAspLeuValArgAspAlaLysAspAlaLeuGluProTyrTrpLysAlaGlu
ThrIleLysLysHisValLeuIleAlaThrHisPheValAspLeuIleGluAspPheTrp
GlnThrThrGlnGlyGlySerGlyAlaThrAsnPheSerLeuLeuLysGlnAlaGlyAsp
ValGluGluAsnProGlyProMetSerGlnIleAlaAspAlaLeuArgAlaValIlePro
ProThrThrThrProValProAspGlyTyrLeuIleSerHisAsnGluAlaGlnGluLeu
ProLeuAsnAspLeuPheValLeuGlnGluGluArgIleValAsnPheGlnProAspTyr
ProIleThrAlaArgIleHisThrHisLeuArgValTyrGlySerGlyAlaThrAsnPhe
SerLeuLeuLysGlnAlaGlyAspValGluGluAsnProGlyProThrLysLeuAsnGlu
GlnAlaLeuAspLysAlaArgArgLeuLeuTrpTrpHisTyrAsnCysLeuLeuTrpGly
GluSerAsnValThrAsnTyrIleSerArgLeuArgThrTrpLeuSerThrProGluLys
TyrArgGlyLysAspAlaProThrIleGluAlaIleThrArgProIleGlnValAlaGln
GlyGlySerGlyAlaThrAsnPheSerLeuLeuLysGlnAlaGlyAspValGluGluAsn
ProGlyProSerArgAsnGlnThrLysGlyValArgLysProArgGlyLeuGluProArg
ArgArgLysValLysThrThrValValTyrGlyArgArgArgSerLysSerArgGlyArg
ArgSerSerProSerGlnArgAlaGlySerProIleProArgAsnArgGluAsnGlnSer
ArgSerSerSerProArgGlu
SEQ ID NO: 29
GSG-P2A-coSHBcAg multiple sites
GGAAGCGGAGCTACTAACTTCAGCCTGCTGAAGCAGGCTGGAGACGTGGAGGAGAACCCTGG
ACCTATGGACGTGAACGCCAGCAGAGCCCTGGCCAACGTGTACGACCTGCCCGACGACTTCT
TCCCCCAGATCGACGACCTGGTGAGAGACGCCAAGGACGCCCTGGAGCCCTACTGGAAGGCC
GAGACCATCAAGAAGCACGTGCTGATCGCCACCCACTTCGTGGACTTGATCGAGGACTTCTG
GCAGACCACCCAGGGCGGAAGCGGAGCTACTAACTTCAGCCTGCTGAAGCAGGCTGGAGACG
TGGAGGAGAACCCTGGACCTATGAGCCAGATCGCCGACGCCCTGAGAGCCGTGATCCCCCCC
ACCACCACCCCCGTGCCCGACGGCTACCTGATCAGCCACAACGAGGCCCAGGAGCTCCCCCT
GAACGACCTGTTCGTGCTGCAGGAGGAGAGAATCGTGAACTTCCAGCCCGACTACCCCATCA
CCGCCAGAATTCACACCCACCTGAGAGTGTACGGAAGCGGAGCTACTAACTTCAGCCTGCTG
AAGCAGGCTGGAGACGTGGAGGAGAACCCTGGACCTACCAAGCTGAACGAGCAGGCCCTGGA
CAAGGCCAGAAGACTGCTGTGGTGGCACTACAACTGCCTGCTGTGGGGCGAGAGCAACGTGA
CCAACTACATCAGCAGACTGAGAACCTGGCTGAGCACCCCCGAGAAGTACAGAGGCAAGGAC
GCCCCCACCATCGAGGCCATCACCAGACCCATCCAGGTGGCCCAGGGCGGAAGCGGAGCTAC
TAACTTCAGCCTGCTGAAGCAGGCTGGAGACGTGGAGGAGAACCCTGGACCTAGCAGAAACC
AGACCAAGGGCGTGAGAAAGCCCAGAGGCCTGGAGCCCAGAAGAAGAAAGGTGAAGACCACC
GTGGTGTACGGCAGAAGAAGAAGCAAGAGCAGAGGCAGAAGAAGCAGCCCCAGCCAGAGAGC
CGGCAGCCCCATCCCCAGAAACAGAGAGAACCAGAGCAGAAGCAGCAGCCCCAGAGAG
SEQ ID NO: 30
GSG-P2A-coSHBcAg multiple sites- polypeptide
GlySerGly
AlaThrAsnPheSerLeuLeuLysGlnAlaGlyAspValGluGluAsnProGlyProMet
AspValAsnAlaSerArgAlaLeuAlaAsnValTyrAspLeuProAspAspPhePhePro
GlnIleAspAspLeuValArgAspAlaLysAspAlaLeuGluProTyrTrpLysAlaGlu
ThrIleLysLysHisValLeuIleAlaThrHisPheValAspLeuIleGluAspPheTrp
GlnThrThrGlnGlyGlySerGlyAlaThrAsnPheSerLeuLeuLysGlnAlaGlyAsp
ValGluGluAsnProGlyProMetSerGlnIleAlaAspAlaLeuArgAlaValIlePro
ProThrThrThrProValProAspGlyTyrLeuIleSerHisAsnGluAlaGlnGluLeu
ProLeuAsnAspLeuPheValLeuGlnGluGluArgIleValAsnPheGlnProAspTyr
ProIleThrAlaArgIleHisThrHisLeuArgValTyrGlySerGlyAlaThrAsnPhe
SerLeuLeuLysGlnAlaGlyAspValGluGluAsnProGlyProThrLysLeuAsnGlu
GlnAlaLeuAspLysAlaArgArgLeuLeuTrpTrpHisTyrAsnCysLeuLeuTrpGly
GluSerAsnValThrAsnTyrIleSerArgLeuArgThrTrpLeuSerThrProGluLys
TyrArgGlyLysAspAlaProThrIleGluAlaIleThrArgProIleGlnValAlaGln
GlyGlySerGlyAlaThrAsnPheSerLeuLeuLysGlnAlaGlyAspValGluGluAsn
ProGlyProSerArgAsnGlnThrLysGlyValArgLysProArgGlyLeuGluProArg
ArgArgLysValLysThrThrValValTyrGlyArgArgArgSerLysSerArgGlyArg
ArgSerSerProSerGlnArgAlaGlySerProIleProArgAsnArgGluAsnGlnSer
ArgSerSerSerProArgGlu
SEQ ID NO: 31
pcDNA3.1 expression vector
GACGGATCGGGAGATCTCCCGATCCCCTATGGTGCACTCTCAGTACAATCTGCTCTGATGCC
GCATAGTTAAGCCAGTATCTGCTCCCTGCTTGTGTGTTGGAGGTCGCTGAGTAGTGCGCGAG
CAAAATTTAAGCTACAACAAGGCAAGGCTTGACCGACAATTGCATGAAGAATCTGCTTAGGG
TTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCG
CGTTACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTAT
ATAAGCAGAGCTCTCTGGCTAACTAGAGAACCCACTGCTTACTGGCTTATCGAAATTAATAC
GACTCACTATAGGGAGACCCAAGCTGGCTAGCGTTTAAACTTAAGCTTGGTACCGAGCTCGG
ATCCACTAGTCCAGTGTGGTGGAATTCTGCAGATATCCAGCACAGTGGCGGCCGCTCGAGTC
TAGAGGGCCCGTTTAAACCCGCTGATCAGCCTCGACTGTGCCTTCTAGTTGCCAGCCATCTG
TTGTTTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGGGGTGG
GGTGGGGCAGGACAGCAAGGGGGAGGATTGGGAAGACAATAGCAGGCATGCTGGGGATGCGG
TGGGCTCTATGGCTTCTGAGGCGGAAAGAACCAGCTGGGGCTCTAGGGGGTATCCCCACGCG
CCCTGTAGCGGCGCATTAAGCGCGGCGGGTGTGGTGGTTACGCGCAGCGTGACCGCTACACT
TGCCAGCGCCCTAGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTTAGTGCTTTACGG
CACCTCGACCCCAAAAAACTTGATTAGGGTGATGGTTCACGTAGTGGGCCATCGCCCTGATA
GACGGTTTTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTTGCCGATT
TCGGCCTATTGGTTAAAAAATGAGCTGATTTAACAAAAATTTAACGCGAATTAATTCTGTGG
AATGTGTGTCAGTTAGGGTGTGGAAAGTCCCCAGGCTCCCCAGCAGGCAGAAGTATGCAAAG
CATGCATCTCAATTAGTCAGCAACCAGGTGTGGAAAGTCCCCAGGCTCCCCAGCAGGCAGAA
GTATGCAAAGCATGCATCTCAATTAGTCAGCAACCATAGTCCCGCCCCTAACTCCGCCCATC
CCGCCCCTAACTCCGCCCAGTTCCGCCCATTCTCCGCCCCATGGCTGACTAATTTTTTTTAT
TTATGCAGAGGCCGAGGCCGCCTCTGCCTCTGAGCTATTCCAGAAGTAGTGAGGAGGCTTTT
TTGGAGGCCTAGGCTTTTGCAAAAAGCTCCCGGGAGCTTGTATATCCATTTTCGGATCTGAT
CAAGAGACAGGATGAGGATCGTTTCGCATGATTGAACAAGATGGATTGCACGCAGGTTCTCC
GGCCGCTTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCGACCTG
TCCGGTGCCCTGAATGAACTGCAGGACGAGGCAGCGCGGCTATCGTGGCTGGCCACGACGGG
CGTTCCTTGCGCAGCTGTGCTCGACGTTGTCACTGAAGCGGGAAGGGACTGGCTGCTATTGG
GCGAAGTGCCGGGGCAGGATCTCCTGTCATCTCACCTTGCTCCTGCCGAGAAAGTATCCATC
ATGGCTGATGCAATGCGGCGGCTGCATACGCTTGATCCGGCTACCTGCCCATTCGACCACCA
AGCGAAACATCGCATCGAGCGAGCACGTACTCGGATGGAAGCCGGTCTTGTCGATCAGGATG
ATCTGGACGAAGAGCATCAGGGGCTCGCGCCAGCCGAACTGTTCGCCAGGCTCAAGGCGCGC
ATGCCCGACGGCGAGGATCTCGTCGTGACCCATGGCGATGCCTGCTTGCCGAATATCATGGT
GGAAAATGGCCGCTTTTCTGGATTCATCGACTGTGGCCGGCTGGGTGTGGCGGACCGCTATC
AGGACATAGCGTTGGCTACCCGTGATATTGCTGAAGAGCTTGGCGGCGAATGGGCTGACCGC
TTCCTCGTGCTTTACGGTATCGCCGCTCCCGATTCGCAGCGCATCGCCTTCTATCGCCTTCT
TGACGAGTTCTTCTGAGCGGGACTCTGGGGTTCGAAATGACCGACCAAGCGACGCCCAACCT
GCCATCACGAGATTTCGATTCCACCGCCGCCTTCTATGAAAGGTTGGGCTTCGGAATCGTTT
TCCGGGACGCCGGCTGGATGATCCTCCAGCGCGGGGATCTCATGCTGGAGTTCTTCGCCCAC
CCCAACTTGTTTATTGCAGCTTATAATGGTTACAAATAAAGCAATAGCATCACAAATTTCAC
AAATAAAGCATTTTTTTCACTGCATTCTAGTTGTGGTTTGTCCAAACTCATCAATGTATCTT
ATCATGTCTGTATACCGTCGACCTCTAGCTAGAGCTTGGCGTAATCATGGTCATAGCTGTTT
CCTGTGTGAAATTGTTATCCGCTCACAATTCCACACAACATACGAGCCGGAAGCATAAAGTG
TAAAGCCTGGGGTGCCTAATGAGTGAGCTAACTCACATTAATTGCGTTGCGCTCACTGCCCG
CTTTCCAGTCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTCGGTCGT
TCGGCTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCACAAAAATCGACG
CTCAAGTCAGAGGTGGCGAAACCCGACAGGACTATAAAGATACCAGGCGTTTCCCCCTGGAA
GCTCCCTCGTGCGCTCTCCTGTTCCGACCCTGCCGCTTACCGGATACCTGTCCGCCTTTCTC
CCTTCGGGAAGCGTGGCGCTTTCTCATAGCTCACGCTGTAGGTATCTCAGTTCGGTGTAGGT
CGTTCGCTCCAAGCTGGGCTGTGTGCACGAACCCCCCGTTCAGCCCGACCGCTGCGCCTTAT
CCGGTAACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAAGTGGTG
GCCTAACTACGGCTACACTAGAAGAACAGTATTTGGTATCTGCGCTCTGCTGAAGCCAGTTA
CCTTCGGAAAAAGAGTTGGTAGCTCTTGATCCGGCAAACAAACCACCGCTGGTAGCGGTTTT
TTTGTTTGCAAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGTCATGAGAT
TATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGA
TACGGGAGGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGC
CAGTTAATAGTTTGCGCAACGTTGTTGCCATTGCTACAGGCATCGTGGTGTCACGCTCGTCG
TTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCG
CAGTGTTATCACTCATGGTTATGGCAGCACTGCATAATTCTCTTACTGTCATGCCATCCGTA
AGATGCTTTTCTGTGACTGGTGAGTACTCAACCAAGTCATTCTGAGAATAGTGTATGCGGCG
ACCGAGTTGCTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTACCGCTGTTG
AGATCCAGTTCGATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGGGAATAAGGGCGA
CACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCC
GCGCACATTTCCCCGAAAAGTGCCACCTGACGTC
SEQ ID NO: 32
pVAX1 expression vector
GACTCTTCGCGATGTACGGGCCAGATATACGCGTTGACATTGATTATTGACTAGTTATTAATAGTAATCA
ATTACGGGGTCATTAGTTCATAGCCCATATATGGAGTTCCGCGTTACATAACTTACGGTAAATGGCCCGC
CTGGCTGACCGCCCAACGACCCCCGCCCATTGACGTCAATAATGACGTATGTTCCCATAGTAACGCCAAT
AGGGACTTTCCATTGACGTCAATGGGTGGACTATTTACGGTAAACTGCCCACTTGGCAGTACATCAAGTG
TATCATATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTCTCCACCCC
ATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAAC
TAGAGAACCCACTGCTTACTGGCTTATCGAAATTAATACGACTCACTATAGGGAGACCCAAGCTGGCTAG
CGTTTAAACTTAAGCTTGGTACCGAGCTCGGATCCACTAGTCCAGTGTGGTGGAATTCTGCAGATATCCA
GCACAGTGGCGGCCGCTCGAGTCTAGAGGGCCCGTTTAAACCCGCTGATCAGCCTCGACTGTGCCTTCTA
GTTGCCAGCCATCTGTTGTTTGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTCATTCTATTCTGGGGGGTGGG
GTGGGGCAGGACAGCAAGGGGGAGGATTGGGAAGACAATAGCAGGCATGCTGGGGATGCGGTGGGCTCTA
TGGCTTCTACTGGGCGGTTTTATGGACAGCAAGCGAACCGGAATTGCCAGCTGGGGCGCCCTCTGGTAAG
GTTGGGAAGCCCTGCAAAGTAAACTGGATGGCTTTCTCGCCGCCAAGGATCTGATGGCGCAGGGGATCAA
GCTCTGATCAAGAGACAGGATGAGGATCGTTTCGCATGATTGAACAAGATGGATTGCACGCAGGTTCTCC
GGCCGCTTGGGTGGAGAGGCTATTCGGCTATGACTGGGCACAACAGACAATCGGCTGCTCTGATGCCGCC
GTGTTCCGGCTGTCAGCGCAGGGGCGCCCGGTTCTTTTTGTCAAGACCGACCTGTCCGGTGCCCTGAATG
AACTGCAAGACGAGGCAGCGCGGCTATCGTGGCTGGCCACGACGGGCGTTCCTTGCGCAGCTGTGCTCGA
CGTTGTCACTGAAGCGGGAAGGGACTGGCTGCTATTGGGCGAAGTGCCGGGGCAGGATCTCCTGTCATCT
CACCTTGCTCCTGCCGAGAAAGTATCCATCATGGCTGATGCAATGCGGCGGCTGCATACGCTTGATCCGG
CTACCTGCCCATTCGACCACCAAGCGAAACATCGCATCGAGCGAGCACGTACTCGGATGGAAGCCGGTCT
TGTCGATCAGGATGATCTGGACGAAGAGCATCAGGGGCTCGCGCCAGCCGAACTGTTCGCCAGGCTCAAG
GCGAGCATGCCCGACGGCGAGGATCTCGTCGTGACCCATGGCGATGCCTGCTTGCCGAATATCATGGTGG
AAAATGGCCGCTTTTCTGGATTCATCGACTGTGGCCGGCTGGGTGTGGCGGACCGCTATCAGGACATAGC
GTTGGCTACCCGTGATATTGCTGAAGAGCTTGGCGGCGAATGGGCTGACCGCTTCCTCGTGCTTTACGGT
ATCGCCGCTCCCGATTCGCAGCGCATCGCCTTCTATCGCCTTCTTGACGAGTTCTTCTGAATTATTAACG
CTTACAATTTCCTGATGCGGTATTTTCTCCTTACGCATCTGTGCGGTATTTCACACCGCATACAGGTGGC
ACTTTTCGGGGAAATGTGCGCGGAACCCCTATTTGTTTATTTTTCTAAATACATTCAAATATGTATCCGC
TCATGAGACAATAACCCTGATAAATGCTTCAATAATAGCACGTGCTAAAACTTCATTTTTAATTTAAAAG
GATCTAGGTGAAGATCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCTGTCGGGTT
TCGCCACCTCTGACTTGAGCGTCGATTTTTGTGATGCTCGTCAGGGGGGCGGAGCCTATGGAAAAACGCC
AGCAACGCGGCCTTTTTACGGTTCCTGGGCTTTTGCTGGCCTTTTGCTCACATGTTCTT
SEQ ID NO: 33
coHDAg-S (gt1)
CGGAGCGAGTCAAAGAGAAATAGGGACGGGAGAGAAGGCATCCTGGAGCAGTGGGTCAATGG
AAGAAAGAAACTGGAAGATCTGGAAAGAGAGGCAAGGAAGATCAAGAAGAAAATCAAAAAAT
TAGAGGATGAAAATCCATGGTTAGGAAACATCAAAGGAATCCTCGGAAAAAGAGACAAGGAT
GGAGAAGGCGCCCCCCCTGCTAAAAGGGCACGCACAGACCAGATGGAGATCGATAGTGGACC
AGGAAAGCGACCTCTGCGAGGAGGGTTCTCAGACAAAGAACGCCAGGATCACCGGAGAAGGA
AGGCCCTGGAGAACAAGCGGAAGCAGCTGGCCGCTGGAGGCAAACATCTGAGCAAGGAGGAA
GAGGAAGAGCTGAAGCGCCTGACCGAAGAGGATGAACGCCGAGAGCGGAGAACAGCTGGACC
ATCCGTGGGAGGAGTCAATCCCCTGGAGGGAGGATCTCGGGGAGCACCAGGAGGAGGGTTCG
TGCCTAACATGCTGAGCGTCCCAGAATCACCCTTTAGCCGAACTGGAGAGGGGCTGGACGTG
AGGGGCAATCAGGGGTTCCCC
SEQ ID NO: 34
HDAg-S (gt1) polypeptide
ArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPhePro
SEQ ID NO: 35
coHDAg-L (gt1)
AGGAGCGAGTCAAAAAGGAACAGGGATGGGAGGGAAGGCATTCTGGAACAGTGGGTCAACGG
ACGGAAAAAACTGGAGGATCTGGAAAGAGAGGCTAGGAAGATCAAGAAGAAAATCAAGAAGT
TAGAGGATGAAAATCCTTGGTTAGGAAACATCAAAGGAATCCTCGGAAAACGCGACAAGGAT
GGAGAAGGCGCCCCCCCTGCTAAAAGGGCACGCACAGACCAGATGGAGATCGATAGCGGACC
AGGAAAGCGACCACTGCGAGGAGGGTTTTCCGACAAAGAACGCCAGGATCACCGGAGAAGGA
AGGCCCTGGAGAACAAGCGGAAGCAGCTGGCCGCTGGAGGCAAACATCTGTCCAAGGAGGAA
GAGGAAGAGCTGAAGCGCCTGACCGAAGAGGACGAACGCCGAGAGCGGAGAACAGCTGGACC
AAGTGTGGGAGGAGTCAATCCCCTGGAGGGAGGATCACGGGGAGCACCTGGAGGAGGATTTG
TGCCAAACATGCTGTCTGTCCCTGAATCACCATTCAGCCGAACAGGCGAGGGGCTGGATGTG
AGGGGCAATCAGGGGTTCCCCTGGGACATCCTGTTTCCAGCAGATCCACCCTTCTCCCCTCA
GTCTTGCAGGCCACAG
SEQ ID NO: 36
HDAg-L (gt1) polypeptide
ArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProTrpAspIleLeuPhe
ProAlaAspProProPheSerProGlnSerCysArgProGln
SEQ ID NO: 37
coHDAg-L (gt2)
CAGCCCGATAGCAGAAGACCTAGAAGAGGGAGGGAAGAAAGCCTGGGGAAATGGATTGACGC
CCGAAGAAGGAAAGAAGAACTGGAGCGAGACCTGCGGAAAGTGAATAAGACAATCAAAAGAC
TGGAGGAAGATAACCCCTGGCTGGGGAATATTCGCGGCATCATTGGGCGAAAAGACAAGGAT
GGAGAAGGAGCTCCACCTGCAAAGCGAGCACGAACAGACCAGATGGAGGTGGATAGCGGCCC
CCGGAAAAGAAAGCACCCTGGCGGGTTCACTGAACAGGAGCGGAGAGACCATAGGCGCCGAA
AGGCCCTGGAGAATAAGAAAAAGCAGCTGAGCTCCGGAGGCAAAGACCTGTCCAGGGAGGAA
GAGGAAGAGCTGCGGAGACTGACTGAAGAGGATGAAAGGCGAGAGCGACGAGTCGCAGGACC
TAGAGTGGGCGACGTGAACCCACTGGATGGAGGACCTAGGGGAGCACCAGGAGGAGGATTCG
TGCCTAGTATGCAGGGAATCCCCGAATCACCTTTTACCAGAAGGGGAGACGGCCTGGATACA
CGCGGCACTCAGGAGTTCCCCTGGGTGAATCCTCAGCCACCACCTCCACGACTGCCACTGCT
GGAATGCACCCCCCAG
SEQ ID NO: 38
HDAg-L (gt2) polypeptide
GlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProTrpValAsnProGln
ProProProProArgLeuProLeuLeuGluCysThrProGln
SEQ ID NO: 39
coHDAg-S (gt2)
CAGCCTGATAGTAGGAGACCACGGAGAGGGAGAGAGGAGTCACTGGGAAAATGGATTGATGC
TCGAAGACGGAAGGAGGAACTGGAGCGAGACCTGCGGAAAGTGAATAAGACTATCAAAAGAC
TGGAGGAAGATAACCCCTGGCTGGGGAATATTCGCGGCATCATTGGGCGAAAAGACAAGGAT
GGAGAAGGAGCTCCACCTGCAAAGCGAGCACGAACTGACCAGATGGAGGTGGATAGCGGCCC
CCGGAAAAGAAAGCACCCTGGCGGGTTCACCGAACAGGAGCGGAGAGACCATAGGCGCCGAA
AGGCCCTGGAGAATAAGAAAAAGCAGCTGAGCTCCGGAGGCAAAGACCTGTCCCGCGAGGAA
GAGGAAGAGCTGCGGAGACTGACCGAAGAGGATGAAAGGCGAGAGCGACGAGTCGCAGGACC
AAGAGTGGGCGACGTGAACCCCCTGGATGGAGGACCAAGGGGAGCACCTGGAGGAGGATTCG
TGCCCAGTATGCAGGGAATCCCAGAATCACCCTTTACAAGAAGGGGAGACGGCCTGGATACA
CGGGGCACTCAGGAGTTCCCT
SEQ ID NO: 40
HDAg-S (gt2) polypeptide
GlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPhePro
SEQ ID NO: 41
Kozak concensus
GCACCATGG
SEQ ID NO: 42
Kozak concensus with fame-conserving bases GG
GCACCATGGGG
SEQ ID NO: 43
Kozak concensus with fame-conserving bases GA
GCACCATGGGA
SEQ ID NO: 44
Kozak concensus with fame-conserving bases GC
GCACCATGGGC
SEQ ID NO: 45
Kozak concensus with fame-conserving bases GT
GCACCATGGGT
SEQ ID NO: 46
Kozak concensus with fame-conserving bases AG
GCACCATGGAG
SEQ ID NO: 47
Kozak concensus with fame-conserving bases AA
GCACCATGGAA
SEQ ID NO: 48
Kozak concensus with fame-conserving bases AC
GCACCATGGAC
SEQ ID NO: 49
Kozak concensus with fame-conserving bases AT
GCACCATGGAT
SEQ ID NO: 50
Kozak concensus with fame-conserving bases CG
GCACCATGGCG
SEQ ID NO: 51
Kozak concensus with fame-conserving bases CA
GCACCATGGAA
SEQ ID NO: 52
Kozak concensus with fame-conserving bases CC
GCACCATGGCC
SEQ ID NO: 53
Kozak concensus with fame-conserving bases CT
GCACCATGGCT
SEQ ID NO: 54
Kozak concensus with fame-conserving bases TG
GCACCATGGTG
SEQ ID NO: 55
Kozak concensus with fame-conserving bases TA
GCACCATGGTA
SEQ ID NO: 56
Kozak concensus with fame-conserving bases TC
GCACCATGGTC
SEQ ID NO: 57
Kozak concensus with fame-conserving bases TT
GCACCATGGTT
SEQ ID NO: 58
L1.1
ATGGGGAGGAGCGAGTCAAAAAGGAACAGGGATGGGAGGGAAGGCATTCTGGAACAGTGGGT
CAACGGACGGAAAAAACTGGAGGATCTGGAAAGAGAGGCTAGGAAGATCAAGAAGAAAATCA
AGAAGTTAGAGGATGAAAATCCTTGGTTAGGAAACATCAAAGGAATCCTCGGAAAACGCGAC
AAGGATGGAGAAGGCGCCCCCCCTGCTAAAAGGGCACGCACAGACCAGATGGAGATCGATAG
CGGACCAGGAAAGCGACCACTGCGAGGAGGGTTTTCCGACAAAGAACGCCAGGATCACCGGA
GAAGGAAGGCCCTGGAGAACAAGCGGAAGCAGCTGGCCGCTGGAGGCAAACATCTGTCCAAG
GAGGAAGAGGAAGAGCTGAAGCGCCTGACCGAAGAGGACGAACGCCGAGAGCGGAGAACAGC
TGGACCAAGTGTGGGAGGAGTCAATCCCCTGGAGGGAGGATCACGGGGAGCACCTGGAGGAG
GATTTGTGCCAAACATGCTGTCTGTCCCTGAATCACCATTCAGCCGAACAGGCGAGGGGCTG
GATGTGAGGGGCAATCAGGGGTTCCCCTGGGACATCCTGTTTCCAGCAGATCCACCCTTCTC
CCCTCAGTCTTGCAGGCCACAGGGAAGCGGAGCTACTAACTTCAGCCTGCTGAAGCAGGCTG
GAGACGTGGAGGAGAACCCTGGACCTATGGATGTCAATGCCAGCAGAGCACTGGCCAACGTG
TACGACCTGCCCGACGATTTCTTTCCTCAGATCGACGATCTGGTGAGGGACGCCAAAGATGC
TCTGGAACCCTATTGGAAGGCAGAGACAATCAAAAAGCACGTGCTGATTGCCACTCATTTTG
TCGACCTGATTGAGGATTTCTGGCAGACCACACAGGGCATGTCTCAGATCGCTGACGCACTG
AGAGCCGTGATTCCTCCAACTACCACACCAGTCCCCGATGGATATCTGATTTCCCATAACGA
GGCCCAAGAATTACCACTCAATGACTTATTCGTCCTGCAGGAGGAAAGAATCGTGAACTTTC
AACCTGACTATCCAATTACTGCTAGAATTCACACCCACTTAAGAGTCTACACCAAGTTAAAC
GAGCAAGCCCTGGACAAGGCCAGGCGCCTGCTGTGGTGGCATTACAACTGTCTGCTGTGGGG
CGAGAGCAACGTGACCAATTATATCTCCCGACTGCGGACATGGCTGTCTACTCCAGAAAAAT
ACAGAGGGAAGGATGCTCCTACTATCGAGGCAATTACCCGACCAATTCAGGTCGCACAGGGA
AGCCGGAACCAGACCAAAGGAGTGCGGAAGCCAAGAGGCCTGGAGCCACGACGGAGAAAAGT
GAAGACTACCGTGGTCTATGGCAGGCGCCGATCTAAGAGTAGAGGGCGGAGAAGCTCCCCTA
GCCAGAGGGCAGGAAGCCCCATCCCCAGAAACAGAGAAAACCAGTCCAGAAGCAGCAGCCCA
AGAGAG
SEQ ID NO: 59
L1.1 polypeptide
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProTrpAspIleLeuPhe
ProAlaAspProProPheSerProGlnSerCysArgProGlnGlySerGlyAlaThrAsn
PheSerLeuLeuLysGlnAlaGlyAspValGluGluAsnProGlyProMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyMetSerGlnIleAlaAspAlaLeuArgAlaValIleProProThrThrThrPro
ValProAspGlyTyrLeuIleSerHisAsnGluAlaGlnGluLeuProLeuAsnAspLeu
PheValLeuGlnGluGluArgIleValAsnPheGlnProAspTyrProIleThrAlaArg
IleHisThrHisLeuArgValTyrThrLysLeuAsnGluGlnAlaLeuAspLysAlaArg
ArgLeuLeuTrpTrpHisTyrAsnCysLeuLeuTrpGlyGluSerAsnValThrAsnTyr
IleSerArgLeuArgThrTrpLeuSerThrProGluLysTyrArgGlyLysAspAlaPro
ThrIleGluAlaIleThrArgProIleGlnValAlaGlnGlySerArgAsnGlnThrLys
GlyValArgLysProArgGlyLeuGluProArgArgArgLysValLysThrThrValVal
TyrGlyArgArgArgSerLysSerArgGlyArgArgSerSerProSerGlnArgAlaGly
SerProIleProArgAsnArgGluAsnGlnSerArgSerSerSerProArgGlu
SEQ ID NO: 60
L1.2
ATGGGGAGGAGTGAGTCAAAACGAAATAGAGATGGCAGGGAAGGGATTCTGGAGCAGTGGGT
CAATGGACGGAAAAAACTGGAGGACCTGGAAAGAGAGGCCAGGAAGATCAAGAAGAAAATCA
AAAAATTAGAGGATGAAAATCCTTGGTTAGGAAACATCAAAGGAATCCTCGGAAAACGAGAC
AAGGATGGAGAAGGAGCACCACCTGCTAAAAGGGCACGCACAGACCAGATGGAGATCGATTC
CGGACCAGGCAAGCGACCACTGCGAGGAGGGTTTTCTGACAAAGAACGCCAGGATCACCGGA
GAAGGAAGGCCCTGGAGAACAAGCGGAAGCAGCTGGCCGCTGGAGGCAAACATCTGAGCAAG
GAGGAAGAGGAAGAGCTGAAGCGCCTGACCGAAGAGGACGAACGCCGAGAGCGGAGAACAGC
TGGACCATCAGTGGGAGGAGTCAATCCCCTGGAGGGAGGAAGCCGGGGAGCACCTGGAGGAG
GGTTTGTGCCAAACATGCTGTCCGTCCCTGAATCACCATTCAGCCGAACCGGGGAGGGACTG
GATGTGAGGGGCAATCAGGGGTTCCCCTGGGACATCCTGTTTCCTGCTGATCCACCCTTCTC
CCCTCAGTCTTGCAGGCCACAGGGAAGCGGAGCTACTAACTTCAGCCTGCTGAAGCAGGCTG
GAGACGTGGAGGAGAACCCTGGACCTATGGATGTCAATGCCAGCAGAGCACTGGCCAACGTG
TACGACCTGCCCGACGATTTCTTTCCTCAGATCGACGATCTGGTGAGGGACGCCAAAGATGC
TCTGGAACCCTATTGGAAGGCAGAGACAATCAAAAAGCACGTGCTGATTGCCACTCATTTTG
TCGACCTGATTGAGGATTTCTGGCAGACCACACAGGGAGGAAGCGGAGCTACTAACTTCAGC
CTGCTGAAGCAGGCTGGAGACGTGGAGGAGAACCCTGGACCTATGAGTCAGATCGCTGATGC
ACTGAGAGCTGTGATTCCTCCAACTACCACACCTGTCCCAGATGGATACTTAATTTCCCATA
ACGAGGCTCAAGAATTACCACTAAATGACTTATTCGTCCTGCAGGAGGAAAGAATCGTGAAC
TTTCAACCTGACTATCCAATTACTGCTAGAATTCACACACATCTGCGCGTCTATGGAAGCGG
AGCTACTAACTTCAGCCTGCTGAAGCAGGCTGGAGACGTGGAGGAGAACCCTGGACCTACCA
AGTTAAATGAACAAGCCCTGGACAAGGCCAGGCGCCTGCTGTGGTGGCATTACAATTGTCTG
CTGTGGGGCGAGTCCAACGTGACCAATTATATCTCTCGACTGCGGACATGGCTGAGTACTCC
AGAAAAATACAGAGGGAAGGACGCTCCTACTATCGAGGCAATTACCAGGCCAATTCAGGTGG
CCCAGGGCGGAAGCGGAGCTACTAACTTCAGCCTGCTGAAGCAGGCTGGAGACGTGGAGGAG
AACCCTGGACCTTCCAGAAATCAGACTAAAGGAGTCCGGAAGCCAAGAGGCCTGGAGCCACG
ACGGAGAAAAGTGAAGACTACCGTGGTCTATGGCAGGCGCCGATCTAAGAGTCGCGGGCGGA
GAAGCTCCCCTTCTCAGCGGGCAGGGTCACCTATCCCACGGAACAGAGAGAACCAGAGCAGA
AGCAGCAGTCCAAGAGAG
SEQ ID NO: 61
L1.2 polypeptide
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProTrpAspIleLeuPhe
ProAlaAspProProPheSerProGlnSerCysArgProGlnGlySerGlyAlaThrAsn
PheSerLeuLeuLysGlnAlaGlyAspValGluGluAsnProGlyProMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyGlySerGlyAlaThrAsnPheSerLeuLeuLysGlnAlaGlyAspValGluGlu
AsnProGlyProMetSerGlnIleAlaAspAlaLeuArgAlaValIleProProThrThr
ThrProValProAspGlyTyrLeuIleSerHisAsnGluAlaGlnGluLeuProLeuAsn
AspLeuPheValLeuGlnGluGluArgIleValAsnPheGlnProAspTyrProIleThr
AlaArgIleHisThrHisLeuArgValTyrGlySerGlyAlaThrAsnPheSerLeuLeu
LysGlnAlaGlyAspValGluGluAsnProGlyProThrLysLeuAsnGluGlnAlaLeu
AspLysAlaArgArgLeuLeuTrpTrpHisTyrAsnCysLeuLeuTrpGlyGluSerAsn
ValThrAsnTyrIleSerArgLeuArgThrTrpLeuSerThrProGluLysTyrArgGly
LysAspAlaProThrIleGluAlaIleThrArgProIleGlnValAlaGlnGlyGlySer
GlyAlaThrAsnPheSerLeuLeuLysGlnAlaGlyAspValGluGluAsnProGlyPro
SerArgAsnGlnThrLysGlyValArgLysProArgGlyLeuGluProArgArgArgLys
ValLysThrThrValValTyrGlyArgArgArgSerLysSerArgGlyArgArgSerSer
ProSerGlnArgAlaGlySerProIleProArgAsnArgGluAsnGlnSerArgSerSer
SerProArgGlu
SEQ ID NO: 62
S1.1
ATGGGGCGGAGCGAGTCAAAGAGAAATAGGGACGGGAGAGAAGGCATCCTGGAGCAGTGGGT
CAATGGAAGAAAGAAACTGGAAGATCTGGAAAGAGAGGCAAGGAAGATCAAGAAGAAAATCA
AAAAATTAGAGGATGAAAATCCATGGTTAGGAAACATCAAAGGAATCCTCGGAAAAAGAGAC
AAGGATGGAGAAGGCGCCCCCCCTGCTAAAAGGGCACGCACAGACCAGATGGAGATCGATAG
TGGACCAGGAAAGCGACCTCTGCGAGGAGGGTTCTCAGACAAAGAACGCCAGGATCACCGGA
GAAGGAAGGCCCTGGAGAACAAGCGGAAGCAGCTGGCCGCTGGAGGCAAACATCTGAGCAAG
GAGGAAGAGGAAGAGCTGAAGCGCCTGACCGAAGAGGATGAACGCCGAGAGCGGAGAACAGC
TGGACCATCCGTGGGAGGAGTCAATCCCCTGGAGGGAGGATCTCGGGGAGCACCAGGAGGAG
GGTTCGTGCCTAACATGCTGAGCGTCCCAGAATCACCCTTTAGCCGAACTGGAGAGGGGCTG
GACGTGAGGGGCAATCAGGGGTTCCCCGGAAGCGGAGCTACTAACTTCAGCCTGCTGAAGCA
GGCTGGAGACGTGGAGGAGAACCCTGGACCTATGGATGTCAATGCCTCCAGAGCACTGGCCA
ACGTGTACGATCTGCCAGACGATTTCTTTCCCCAGATCGACGATCTGGTGAGGGACGCCAAA
GATGCTCTGGAACCTTATTGGAAGGCAGAGACAATCAAAAAGCACGTGCTGATTGCCACTCA
TTTCGTCGACCTGATTGAGGATTTTTGGCAGACCACACAGGGCATGAGCCAGATCGCTGACG
CACTGAGAGCCGTGATTCCACCCACTACCACACCCGTCCCTGATGGATACTTAATTAGCCAT
AACGAGGCCCAAGAATTACCTTTAAATGACTTATTTGTCCTGCAGGAGGAAAGAATCGTGAA
CTTCCAACCAGACTATCCTATTACTGCCAGAATTCACACCCACTTAAGAGTCTACACCAAGT
TAAACGAGCAAGCCCTGGACAAGGCCAGGCGCCTGCTGTGGTGGCATTACAACTGCCTGCTG
TGGGGCGAGAGTAACGTGACTAATTATATCTCACGACTGCGGACATGGCTGAGCACTCCAGA
AAAATACCGGGGGAAGGATGCTCCTACTATCGAGGCAATTACCCGCCCAATTCAGGTCGCCC
AGGGCTCTCGGAACCAGACCAAAGGAGTGCGGAAGCCTAGAGGCCTGGAGCCACGACGGAGA
AAAGTGAAGACTACCGTGGTCTATGGCAGGCGCCGATCTAAGAGTAGAGGGCGGAGAAGCTC
CCCCAGTCAGAGGGCAGGGTCACCAATCCCAAGAAACAGAGAAAATCAGAGCAGGTCCTCCA
GCCCACGAGAA
SEQ ID NO: 63
S1.1 polypeptide
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProGlySerGlyAlaThr
AsnPheSerLeuLeuLysGlnAlaGlyAspValGluGluAsnProGlyProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyMetSerGlnIleAlaAspAlaLeuArgAlaValIleProProThrThrThr
ProValProAspGlyTyrLeuIleSerHisAsnGluAlaGlnGluLeuProLeuAsnAsp
LeuPheValLeuGlnGluGluArgIleValAsnPheGlnProAspTyrProIleThrAla
ArgIleHisThrHisLeuArgValTyrThrLysLeuAsnGluGlnAlaLeuAspLysAla
ArgArgLeuLeuTrpTrpHisTyrAsnCysLeuLeuTrpGlyGluSerAsnValThrAsn
TyrIleSerArgLeuArgThrTrpLeuSerThrProGluLysTyrArgGlyLysAspAla
ProThrIleGluAlaIleThrArgProIleGlnValAlaGlnGlySerArgAsnGlnThr
LysGlyValArgLysProArgGlyLeuGluProArgArgArgLysValLysThrThrVal
ValTyrGlyArgArgArgSerLysSerArgGlyArgArgSerSerProSerGlnArgAla
GlySerProIleProArgAsnArgGluAsnGlnSerArgSerSerSerProArgGlu
SEQ ID NO: 64
S1.2
ATGGGACGGAGTGAGTCAAAGAGAAATAGAGACGGACGGGAGGGCATCCTGGAGCAGTGGGT
CAATGGACGGAAGAAACTGGAAGACCTGGAAAGAGAGGCTAGGAAGATCAAGAAGAAAATCA
AAAAATTAGAGGATGAAAATCCATGGTTAGGAAACATCAAAGGAATCCTCGGAAAAAGAGAC
AAGGATGGCGAAGGGGCCCCCCCTGCTAAAAGGGCACGCACAGACCAGATGGAGATCGATTC
AGGACCAGGCAAGCGACCTCTGCGAGGAGGGTTTAGCGACAAAGAACGCCAGGATCACCGGA
GAAGGAAGGCCCTGGAGAACAAGCGGAAGCAGCTGGCCGCTGGAGGCAAACATCTGTCCAAG
GAGGAAGAGGAAGAGCTGAAGCGCCTGACCGAAGAGGATGAACGCCGAGAGCGGAGAACAGC
TGGACCATCTGTGGGAGGAGTCAATCCCCTGGAGGGAGGAAGTCGGGGAGCACCAGGAGGAG
GGTTCGTGCCTAACATGCTGTCTGTCCCAGAATCACCCTTTAGCCGAACTGGGGAGGGACTG
GACGTGAGGGGCAATCAGGGGTTCCCCGGAAGCGGAGCTACTAACTTCAGCCTGCTGAAGCA
GGCTGGAGACGTGGAGGAGAACCCTGGACCTATGGATGTCAATGCCAGCAGAGCACTGGCCA
ACGTGTACGATCTGCCAGACGATTTCTTTCCCCAGATCGACGATCTGGTGAGGGACGCCAAA
GATGCTCTGGAACCTTATTGGAAGGCAGAGACAATCAAAAAGCACGTGCTGATTGCCACTCA
TTTCGTCGACCTGATTGAGGATTTTTGGCAGACCACACAGGGAGGAAGCGGAGCTACTAACT
TCAGCCTGCTGAAGCAGGCTGGAGACGTGGAGGAGAACCCTGGACCTATGTCCCAGATCGCT
GATGCACTGAGAGCTGTGATTCCACCCACTACCACACCAGTCCCCGACGGATACTTAATTTC
CCATAACGAGGCTCAAGAATTACCTTTAAATGACTTATTTGTCCTGCAGGAGGAAAGAATCG
TGAACTTCCAACCAGACTATCCTATTACTGCTAGAATTCACACACATCTGCGCGTCTATGGA
AGCGGAGCTACTAACTTCAGCCTGCTGAAGCAGGCTGGAGACGTGGAGGAGAACCCTGGACC
TACCAAGTTAAATGAACAAGCCCTGGACAAGGCCAGGCGCCTGCTGTGGTGGCATTACAATT
GCCTGCTGTGGGGCGAGTCAAACGTGACCAATTATATCAGCCGACTGCGGACATGGCTGTCC
ACTCCAGAAAAATACAGAGGGAAGGACGCTCCTACTATCGAGGCAATTACCAGGCCAATTCA
GGTGGCCCAGGGGGGAAGCGGAGCTACTAACTTCAGCCTGCTGAAGCAGGCTGGAGACGTGG
AGGAGAACCCTGGACCTAGTCGGAATCAGACAAAAGGAGTCCGGAAGCCTAGAGGCCTGGAG
CCACGACGGAGAAAAGTGAAGACTACCGTGGTCTATGGCAGGCGCCGATCTAAGAGTCGCGG
GCGGAGAAGCTCCCCCAGCCAGCGGGCAGGCAGCCCAATCCCCCGCAACAGAGAAAACCAGT
CCCGCTCCTCCTCACCACGAGAG
SEQ ID NO: 65
S1.2 polypeptide
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProGlySerGlyAlaThr
AsnPheSerLeuLeuLysGlnAlaGlyAspValGluGluAsnProGlyProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyGlySerGlyAlaThrAsnPheSerLeuLeuLysGlnAlaGlyAspValGlu
GluAsnProGlyProMetSerGlnIleAlaAspAlaLeuArgAlaValIleProProThr
ThrThrProValProAspGlyTyrLeuIleSerHisAsnGluAlaGlnGluLeuProLeu
AsnAspLeuPheValLeuGlnGluGluArgIleValAsnPheGlnProAspTyrProIle
ThrAlaArgIleHisThrHisLeuArgValTyrGlySerGlyAlaThrAsnPheSerLeu
LeuLysGlnAlaGlyAspValGluGluAsnProGlyProThrLysLeuAsnGluGlnAla
LeuAspLysAlaArgArgLeuLeuTrpTrpHisTyrAsnCysLeuLeuTrpGlyGluSer
AsnValThrAsnTyrIleSerArgLeuArgThrTrpLeuSerThrProGluLysTyrArg
GlyLysAspAlaProThrIleGluAlaIleThrArgProIleGlnValAlaGlnGlyGly
SerGlyAlaThrAsnPheSerLeuLeuLysGlnAlaGlyAspValGluGluAsnProGly
ProSerArgAsnGlnThrLysGlyValArgLysProArgGlyLeuGluProArgArgArg
LysValLysThrThrValValTyrGlyArgArgArgSerLysSerArgGlyArgArgSer
SerProSerGlnArgAlaGlySerProIleProArgAsnArgGluAsnGlnSerArgSer
SerSerProArgGlu
SEQ ID NO: 66
L2.1
ATGGGACAGCCCGATAGCAGAAGACCTAGAAGAGGGAGGGAAGAAAGCCTGGGGAAATGGAT
TGACGCCCGAAGAAGGAAAGAAGAACTGGAGCGAGACCTGCGGAAAGTGAATAAGACAATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACAGACCAGATGGAGGTGGATAG
CGGCCCCCGGAAAAGAAAGCACCCTGGCGGGTTCACTGAACAGGAGCGGAGAGACCATAGGC
GCCGAAAGGCCCTGGAGAATAAGAAAAAGCAGCTGAGCTCCGGAGGCAAAGACCTGTCCAGG
GAGGAAGAGGAAGAGCTGCGGAGACTGACTGAAGAGGATGAAAGGCGAGAGCGACGAGTCGC
AGGACCTAGAGTGGGCGACGTGAACCCACTGGATGGAGGACCTAGGGGAGCACCAGGAGGAG
GATTCGTGCCTAGTATGCAGGGAATCCCCGAATCACCTTTTACCAGAAGGGGAGACGGCCTG
GATACACGCGGCACTCAGGAGTTCCCCTGGGTGAATCCTCAGCCACCACCTCCACGACTGCC
ACTGCTGGAATGCACCCCCCAGGGAAGCGGAGCTACTAACTTCAGCCTGCTGAAGCAGGCTG
GAGACGTGGAGGAGAACCCTGGACCTATGGATGTCAATGCCAGTAGAGCCCTGGCTAACGTG
TACGACCTGCCAGACGATTTCTTTCCCCAGATCGACGATCTGGTGAGGGACGCTAAGGATGC
ACTGGAACCATATTGGAAAGCCGAGACTATCAAAAAGCACGTGCTGATTGCTACCCATTTCG
TCGACCTGATTGAGGATTTTTGGCAGACCACACAGGGAATGAGTCAGATCGCAGACGCACTG
CGAGCTGTGATTCCACCTACTACCACACCTGTCCCAGATGGATACTTAATTTCCCATAACGA
GGCCCAAGAATTACCTTTAAATGACTTATTTGTCCTGCAGGAGGAAAGAATCGTGAACTTCC
AACCTGACTATCCTATTACCGCCAGAATTCACACTCACTTAAGAGTGTACACTAAACTCAAC
GAACAAGCCCTGGACAAGGCCCGCCGACTGCTGTGGTGGCATTACAACTGTCTGCTGTGGGG
CGAAAGCAACGTGACAAATTATATCTCCAGGCTGCGCACCTGGCTGTCTACACCTGAAAAGT
ACCGGGGCAAAGATGCACCCACAATCGAGGCCATTACTAGACCTATTCAGGTCGCCCAGGGG
TCTAGGAACCAGACCAAGGGGGTGAGAAAACCTAGGGGACTGGAGCCACGGAGAAGGAAGGT
GAAGACTACCGTGGTCTATGGACGCCGACGGTCAAAGAGCCGCGGCAGAAGGTCTAGTCCAA
GCCAGCGGGCAGGGTCACCAATCCCCAGAAACCGAGAGAATCAGTCCAGGTCATCATCACCA
AGAGAA
SEQ ID NO: 67
L2.1 polypeptide
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProTrpValAsnProGln
ProProProProArgLeuProLeuLeuGluCysThrProGlnGlySerGlyAlaThrAsn
PheSerLeuLeuLysGlnAlaGlyAspValGluGluAsnProGlyProMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyMetSerGlnIleAlaAspAlaLeuArgAlaValIleProProThrThrThrPro
ValProAspGlyTyrLeuIleSerHisAsnGluAlaGlnGluLeuProLeuAsnAspLeu
PheValLeuGlnGluGluArgIleValAsnPheGlnProAspTyrProIleThrAlaArg
IleHisThrHisLeuArgValTyrThrLysLeuAsnGluGlnAlaLeuAspLysAlaArg
ArgLeuLeuTrpTrpHisTyrAsnCysLeuLeuTrpGlyGluSerAsnValThrAsnTyr
IleSerArgLeuArgThrTrpLeuSerThrProGluLysTyrArgGlyLysAspAlaPro
ThrIleGluAlaIleThrArgProIleGlnValAlaGlnGlySerArgAsnGlnThrLys
GlyValArgLysProArgGlyLeuGluProArgArgArgLysValLysThrThrValVal
TyrGlyArgArgArgSerLysSerArgGlyArgArgSerSerProSerGlnArgAlaGly
SerProIleProArgAsnArgGluAsnGlnSerArgSerSerSerProArgGlu
SEQ ID NO: 68
L2.2
ATGGGACAGCCTGATTCACGGAGACCACGGAGGGGCAGAGAGGAGTCACTGGGGAAATGGAT
TGATGCTCGCAGAAGAAAAGAAGAACTGGAGCGAGACCTGCGGAAAGTGAATAAGACCATCA
AAAGACTGGAGGAAGATAACCCCTGGCTGGGCAATATTCGCGGCATCATTGGGCGAAAAGAC
AAGGATGGAGAAGGAGCTCCACCTGCAAAGCGAGCACGAACCGACCAGATGGAGGTGGATTC
CGGCCCCCGGAAAAGAAAGCACCCTGGCGGGTTTACAGAACAGGAGCGGAGAGACCATAGGC
GCCGAAAGGCCCTGGAGAATAAGAAAAAGCAGCTGAGCTCCGGAGGCAAAGACCTGTCTAGG
GAGGAAGAGGAAGAGCTGCGGAGACTGACAGAAGAGGATGAAAGGCGAGAGCGACGAGTCGC
AGGACCTCGAGTGGGCGACGTGAACCCACTGGATGGAGGACCTCGAGGAGCACCAGGAGGAG
GATTCGTGCCTTCAATGCAGGGAATCCCCGAAAGCCCTTTTACTAGAAGGGGCGACGGGCTG
GATACACGAGGAACTCAGGAGTTCCCCTGGGTGAATCCTCAGCCACCACCTCCACGACTGCC
ACTGCTGGAATGCACCCCCCAGGGAAGCGGAGCTACTAACTTCAGCCTGCTGAAGCAGGCTG
GAGACGTGGAGGAGAACCCTGGACCTATGGATGTCAATGCCTCCAGAGCCCTGGCTAACGTG
TACGACCTGCCAGACGATTTCTTTCCCCAGATCGACGATCTGGTGAGGGACGCTAAGGATGC
ACTGGAACCATATTGGAAAGCCGAGACTATCAAAAAGCACGTGCTGATTGCTACCCATTTCG
TCGACCTGATTGAGGATTTTTGGCAGACCACACAGGGCGGAAGCGGAGCTACTAACTTCAGC
CTGCTGAAGCAGGCTGGAGACGTGGAGGAGAACCCTGGACCTATGTCACAGATCGCAGATGC
CCTGCGCGCTGTGATTCCCCCTACTACCACACCCGTCCCTGATGGATACTTAATTTCCCATA
ACGAGGCTCAAGAATTACCTTTAAATGACTTATTTGTCCTGCAGGAGGAAAGAATCGTGAAC
TTCCAACCTGACTATCCTATTACCGCCAGAATTCACACCCATCTGCGGGTCTATGGAAGCGG
AGCTACTAACTTCAGCCTGCTGAAGCAGGCTGGAGACGTGGAGGAGAACCCTGGACCTACCA
AACTCAATGAACAAGCCCTGGACAAGGCCCGCCGACTGCTGTGGTGGCATTACAATTGTCTG
CTGTGGGGCGAGTCCAACGTGACCAATTATATCTCTAGGCTGCGCACCTGGCTGAGTACACC
TGAAAAGTACAGAGGCAAAGACGCACCCACAATCGAGGCCATTACTAGGCCTATTCAGGTCG
CTCAGGGAGGAAGCGGAGCTACTAACTTCAGCCTGCTGAAGCAGGCTGGAGACGTGGAGGAG
AACCCTGGACCTAGTCGGAATCAGACCAAGGGGGTCAGAAAACCTAGGGGACTGGAGCCACG
GAGAAGGAAGGTGAAGACTACCGTGGTCTATGGACGCCGACGGTCAAAAAGCAGAGGCAGAA
GGTCTAGTCCAAGCCAGAGGGCAGGCAGCCCTATCCCACGGAACAGAGAAAACCAGAGCAGG
TCCAGCAGTCCAAGAGAA
SEQ ID NO: 69
L2.2 polypeptide
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProTrpValAsnProGln
ProProProProArgLeuProLeuLeuGluCysThrProGlnGlySerGlyAlaThrAsn
PheSerLeuLeuLysGlnAlaGlyAspValGluGluAsnProGlyProMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyGlySerGlyAlaThrAsnPheSerLeuLeuLysGlnAlaGlyAspValGluGlu
AsnProGlyProMetSerGlnIleAlaAspAlaLeuArgAlaValIleProProThrThr
ThrProValProAspGlyTyrLeuIleSerHisAsnGluAlaGlnGluLeuProLeuAsn
AspLeuPheValLeuGlnGluGluArgIleValAsnPheGlnProAspTyrProIleThr
AlaArgIleHisThrHisLeuArgValTyrGlySerGlyAlaThrAsnPheSerLeuLeu
LysGlnAlaGlyAspValGluGluAsnProGlyProThrLysLeuAsnGluGlnAlaLeu
AspLysAlaArgArgLeuLeuTrpTrpHisTyrAsnCysLeuLeuTrpGlyGluSerAsn
ValThrAsnTyrIleSerArgLeuArgThrTrpLeuSerThrProGluLysTyrArgGly
LysAspAlaProThrIleGluAlaIleThrArgProIleGlnValAlaGlnGlyGlySer
GlyAlaThrAsnPheSerLeuLeuLysGlnAlaGlyAspValGluGluAsnProGlyPro
SerArgAsnGlnThrLysGlyValArgLysProArgGlyLeuGluProArgArgArgLys
ValLysThrThrValValTyrGlyArgArgArgSerLysSerArgGlyArgArgSerSer
ProSerGlnArgAlaGlySerProIleProArgAsnArgGluAsnGlnSerArgSerSer
SerProArgGlu
SEQ ID NO: 70
S2.1
ATGGGACAGCCTGATAGTAGGAGACCACGGAGAGGGAGAGAGGAGTCACTGGGAAAATGGAT
TGATGCTCGAAGACGGAAGGAGGAACTGGAGCGAGACCTGCGGAAAGTGAATAAGACTATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACTGACCAGATGGAGGTGGATAG
CGGCCCCCGGAAAAGAAAGCACCCTGGCGGGTTCACCGAACAGGAGCGGAGAGACCATAGGC
GCCGAAAGGCCCTGGAGAATAAGAAAAAGCAGCTGAGCTCCGGAGGCAAAGACCTGTCCCGC
GAGGAAGAGGAAGAGCTGCGGAGACTGACCGAAGAGGATGAAAGGCGAGAGCGACGAGTCGC
AGGACCAAGAGTGGGCGACGTGAACCCCCTGGATGGAGGACCAAGGGGAGCACCTGGAGGAG
GATTCGTGCCCAGTATGCAGGGAATCCCAGAATCACCCTTTACAAGAAGGGGAGACGGCCTG
GATACACGGGGCACTCAGGAGTTCCCTGGAAGCGGAGCTACTAACTTCAGCCTGCTGAAGCA
GGCTGGAGACGTGGAGGAGAACCCTGGACCTATGGATGTCAATGCCAGTCGCGCCCTGGCTA
ACGTGTACGACCTGCCAGACGATTTCTTTCCCCAGATCGACGATCTGGTGCGGGACGCTAAG
GATGCACTGGAACCATATTGGAAAGCCGAGACCATCAAAAAGCACGTGCTGATTGCTACACA
TTTCGTCGACCTGATTGAGGATTTTTGGCAGACCACACAGGGAATGAGTCAGATCGCAGACG
CCCTGAGAGCTGTGATTCCACCCACTACCACACCCGTCCCTGATGGATACTTAATTTCCCAT
AACGAGGCCCAAGAATTACCTTTAAATGACTTATTTGTCCTGCAGGAGGAAAGAATCGTGAA
CTTCCAACCAGACTATCCTATTACCGCCAGAATTCACACTCACTTAAGAGTCTACACTAAAC
TCAACGAGCAAGCCCTGGACAAGGCCCGCCGACTGCTGTGGTGGCATTACAACTGCCTGCTG
TGGGGCGAGAGCAACGTGACAAATTATATCTCCAGGCTGCGCACCTGGCTGTCTACACCTGA
AAAGTACAGGGGCAAAGATGCACCTACAATCGAGGCCATTACTAGACCAATTCAGGTCGCCC
AGGGGTCTAGGAACCAGACAAAGGGGGTGAGAAAACCTAGGGGACTGGAGCCACGGAGAAGG
AAGGTGAAGACTACCGTGGTCTATGGACGCCGACGGTCAAAGAGCCGCGGCAGAAGGTCTAG
TCCCAGCCAGCGGGCAGGCAGCCCAATCCCACGCAACAGAGAAAATCAGTCACGGTCCAGCA
GCCCCAGAGAA
SEQ ID NO: 71
S2.1 polypeptide
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProGlySerGlyAlaThr
AsnPheSerLeuLeuLysGlnAlaGlyAspValGluGluAsnProGlyProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyMetSerGlnIleAlaAspAlaLeuArgAlaValIleProProThrThrThr
ProValProAspGlyTyrLeuIleSerHisAsnGluAlaGlnGluLeuProLeuAsnAsp
LeuPheValLeuGlnGluGluArgIleValAsnPheGlnProAspTyrProIleThrAla
ArgIleHisThrHisLeuArgValTyrThrLysLeuAsnGluGlnAlaLeuAspLysAla
ArgArgLeuLeuTrpTrpHisTyrAsnCysLeuLeuTrpGlyGluSerAsnValThrAsn
TyrIleSerArgLeuArgThrTrpLeuSerThrProGluLysTyrArgGlyLysAspAla
ProThrIleGluAlaIleThrArgProIleGlnValAlaGlnGlySerArgAsnGlnThr
LysGlyValArgLysProArgGlyLeuGluProArgArgArgLysValLysThrThrVal
ValTyrGlyArgArgArgSerLysSerArgGlyArgArgSerSerProSerGlnArgAla
GlySerProIleProArgAsnArgGluAsnGlnSerArgSerSerSerProArgGlu
SEQ ID NO: 72
S2.2
ATGGGGCAGCCTGATTCACGGAGACCACGGAGAGGAAGAGAGGAGAGCCTGGGGAAATGGAT
TGACGCACGGAGACGGAAAGAGGAGCTGGAGCGAGACCTGCGGAAAGTGAATAAGACAATCA
AAAGACTGGAGGAAGATAACCCCTGGCTGGGCAATATTCGCGGCATCATTGGGCGAAAAGAC
AAGGATGGAGAAGGAGCTCCACCTGCAAAGCGAGCACGAACAGACCAGATGGAGGTGGATTC
CGGCCCCCGGAAAAGAAAGCACCCTGGCGGGTTTACTGAACAGGAGCGGAGAGACCATAGGC
GCCGAAAGGCCCTGGAGAATAAGAAAAAGCAGCTGAGCTCCGGAGGCAAAGACCTGTCTCGC
GAGGAAGAGGAAGAGCTGCGGAGACTGACCGAAGAGGATGAAAGGCGAGAGCGACGAGTGGC
AGGACCACGAGTGGGCGACGTGAACCCCCTGGATGGAGGACCACGAGGAGCACCTGGAGGAG
GATTCGTGCCCTCAATGCAGGGAATCCCAGAAAGCCCCTTTACCAGAAGGGGCGACGGGCTG
GATACACGAGGAACTCAGGAGTTCCCTGGAAGCGGAGCTACTAACTTCAGCCTGCTGAAGCA
GGCTGGAGACGTGGAGGAGAACCCTGGACCTATGGATGTCAATGCCTCCCGCGCCCTGGCTA
ACGTGTACGACCTGCCAGACGATTTCTTTCCCCAGATCGACGATCTGGTGCGGGACGCTAAG
GATGCACTGGAACCATATTGGAAAGCCGAGACCATCAAAAAGCACGTGCTGATTGCTACACA
TTTCGTCGACCTGATTGAGGATTTTTGGCAGACCACACAGGGCGGAAGCGGAGCTACTAACT
TCAGCCTGCTGAAGCAGGCTGGAGACGTGGAGGAGAACCCTGGACCTATGTCACAGATCGCA
GATGCCCTGAGAGCTGTGATTCCACCCACTACCACACCAGTCCCCGATGGATACTTAATTTC
CCATAACGAGGCTCAAGAATTACCTTTAAATGACTTATTTGTCCTGCAGGAGGAAAGAATCG
TGAACTTCCAACCAGACTATCCTATTACCGCCAGAATTCACACCCATCTGCGGGTCTATGGA
AGCGGAGCTACTAACTTCAGCCTGCTGAAGCAGGCTGGAGACGTGGAGGAGAACCCTGGACC
TACCAAACTCAATGAGCAAGCCCTGGACAAGGCCCGCCGACTGCTGTGGTGGCATTACAATT
GCCTGCTGTGGGGCGAGTCCAACGTGACCAATTATATCTCTAGGCTGCGCACCTGGCTGAGT
ACACCTGAAAAGTACAGAGGCAAAGACGCACCTACAATCGAGGCCATTACTAGGCCAATTCA
GGTCGCTCAGGGAGGAAGCGGAGCTACTAACTTCAGCCTGCTGAAGCAGGCTGGAGACGTGG
AGGAGAACCCTGGACCTAGTAGGAATCAGACCAAGGGGGTCAGAAAACCTAGGGGACTGGAG
CCACGGAGAAGGAAGGTGAAGACTACCGTGGTCTATGGACGCCGACGGTCAAAAAGCAGAGG
CAGAAGGTCTAGTCCCAGCCAGAGGGCAGGGTCACCTATCCCCAGAAACAGAGAAAACCAGT
CAAGGAGTTCATCCCCACGCGAG
SEQ ID NO: 73
S2.2 polypeptide
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProGlySerGlyAlaThr
AsnPheSerLeuLeuLysGlnAlaGlyAspValGluGluAsnProGlyProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyGlySerGlyAlaThrAsnPheSerLeuLeuLysGlnAlaGlyAspValGlu
GluAsnProGlyProMetSerGlnIleAlaAspAlaLeuArgAlaValIleProProThr
ThrThrProValProAspGlyTyrLeuIleSerHisAsnGluAlaGlnGluLeuProLeu
AsnAspLeuPheValLeuGlnGluGluArgIleValAsnPheGlnProAspTyrProIle
ThrAlaArgIleHisThrHisLeuArgValTyrGlySerGlyAlaThrAsnPheSerLeu
LeuLysGlnAlaGlyAspValGluGluAsnProGlyProThrLysLeuAsnGluGlnAla
LeuAspLysAlaArgArgLeuLeuTrpTrpHisTyrAsnCysLeuLeuTrpGlyGluSer
AsnValThrAsnTyrIleSerArgLeuArgThrTrpLeuSerThrProGluLysTyrArg
GlyLysAspAlaProThrIleGluAlaIleThrArgProIleGlnValAlaGlnGlyGly
SerGlyAlaThrAsnPheSerLeuLeuLysGlnAlaGlyAspValGluGluAsnProGly
ProSerArgAsnGlnThrLysGlyValArgLysProArgGlyLeuGluProArgArgArg
LysValLysThrThrValValTyrGlyArgArgArgSerLysSerArgGlyArgArgSer
SerProSerGlnArgAlaGlySerProIleProArgAsnArgGluAsnGlnSerArgSer
SerSerProArgGlu
SEQ ID NO: 74
IL-12
atgtggcccc ctgggtcagc ctcccagcca ccgccctcac ctgccgcggc cacaggtctg
catccagcgg ctcgccctgt gtccctgcag tgccggctca gcatgtgtcc agcgcgcagc
ctcctccttg tggctaccct ggtcctcctg gaccacctca gtttggccag aaacctcccc
gtggccactc cagacccagg aatgttccca tgccttcacc actcccaaaa cctgctgagg
gccgtcagca acatgctcca gaaggccaga caaactctag aattttaccc ttgcacttct
gaagagattg atcatgaaga tatcacaaaa gataaaacca gcacagtgga ggcctgttta
ccattggaat taaccaagaa tgagagttgc ctaaattcca gagagacctc tttcataact
aatgggagtt gcctggcctc cagaaagacc tcttttatga tggccctgtg ccttagtagt
atttatgaag acttgaagat gtaccaggtg gagttcaaga ccatgaatgc aaagcttctg
atggatccta agaggcagat ctttctagat caaaacatgc tggcagttat tgatgagctg
atgcaggccc tgaatttcaa cagtgagact gtgccacaaa aatcctccct tgaagaaccg
gatttttata aaactaaaat caagctctgc atacttcttc atgctttcag aattcgggca
gtgactattg atagagtgat gagctatctg aatgcttcct aa
SEQ ID NO: 75
IL-12 polypeptide
MetTrpProProGlySerAlaSerGlnProProProSerProAlaAlaAlaThrGlyLeu
HisProAlaAlaArgProValSerLeuGlnCysArgLeuSerMetCysProAlaArgSer
LeuLeuLeuValAlaThrLeuValLeuLeuAspHisLeuSerLeuAlaArgAsnLeuPro
ValAlaThrProAspProGlyMetPheProCysLeuHisHisSerGlnAsnLeuLeuArg
AlaValSerAsnMetLeuGlnLysAlaArgGlnThrLeuGluPheTyrProCysThrSer
GluGluIleAspHisGluAspIleThrLysAspLysThrSerThrValGluAlaCysLeu
ProLeuGluLeuThrLysAsnGluSerCysLeuAsnSerArgGluThrSerPheIleThr
AsnGlySerCysLeuAlaSerArgLysThrSerPheMetMetAlaLeuCysLeuSerSer
IleTyrGluAspLeuLysMetTyrGlnValGluPheLysThrMetAsnAlaLysLeuLeu
MetAspProLysArgGlnIlePheLeuAspGlnAsnMetLeuAlaValIleAspGluLeu
MetGlnAlaLeuAsnPheAsnSerGluThrValProGlnLysSerSerLeuGluGluPro
AspPheTyrLysThrLysIleLysLeuCysIleLeuLeuHisAlaPheArgIleArgAla
ValThrIleAspArgValMetSerTyrLeuAsnAlaSer
SEQ ID NO: 76
IL-15
    atggtatt gggaaccata
gatttgtgca gctgtttcag tgcagggctt cctaaaacag aagccaactg ggtgaatgta
ataagtgatt tgaaaaaaat tgaagatctt attcaatcta tgcatattga tgctacttta
tatacggaaa gtgatgttca ccccagttgc aaagtaacag caatgaagtg ctttctcttg
gagttacaag ttatttcact tgagtccgga gatgcaagta ttcatgatac agtagaaaat
ctgatcatcc tagcaaacaa cagtttgtct tctaatggga atgtaacaga atctggatgc
aaagaatgtg aggaactgga ggaaaaaaat attaaagaat ttttgcagag ttttgtacat
attgtccaaa tgttcatcaa cacttcttga
SEQ ID NO: 77
IL-15 polypeptide
MetArgIleSerLysProHisLeuArgSerIleSerIleGlnCysTyrLeuCysLeuLeu
LeuAsnSerHisPheLeuThrGluAlaGlyIleHisValPheIleLeuGlyCysPheSer
AlaGlyLeuProLysThrGluAlaAsnTrpValAsnValIleSerAspLeuLysLysIle
GluAspLeuIleGlnSerMetHisIleAspAlaThrLeuTyrThrGluSerAspValHis
ProSerCysLysValThrAlaMetLysCysPheLeuLeuGluLeuGlnValIleSerLeu
GluSerGlyAspAlaSerIleHisAspThrValGluAsnLeuIleIleLeuAlaAsnAsn
SerLeuSerSerAsnGlyAsnValThrGluSerGlyCysLysGluCysGluGluLeuGlu
GluLysAsnIleLysGluPheLeuGlnSerPheValHisIleValGlnMetPheIleAsn
ThrSer
SEQ ID NO: 78
IL-21
          atgag atccagtcct
ggcaacatgg agaggattgt catctgtctg atggtcatct tcttggggac actggtccac
aaatcaagct cccaaggtca agatcgccac atgattagaa tgcgtcaact tatagatatt
gttgatcagc tgaaaaatta tgtgaatgac ttggtccctg aatttctgcc agctccagaa
gatgtagaga caaactgtga gtggtcagct ttttcctgtt ttcagaaggc ccaactaaag
tcagcaaata caggaaacaa tgaaaggata atcaatgtat caattaaaaa gctgaagagg
aaaccacctt ccacaaatgc agggagaaga cagaaacaca gactaacatg cccttcatgt
gattcttatg agaaaaaacc acccaaagaa ttcctagaaa gattcaaatc acttctccaa
aagatgattc atcagcatct gtcctctaga acacacggaa gtgaagattc ctga
SEQ ID NO: 79
IL-21 polypeptide
MetArgSerSerProGlyAsnMetGluArgIleValIleCysLeuMetValIlePheLeu
GlyThrLeuValHisLysSerSerSerGlnGlyGlnAspArgHisMetIleArgMetArg
GlnLeuIleAspIleValAspGlnLeuLysAsnTyrValAsnAspLeuValProGluPhe
LeuProAlaProGluAspValGluThrAsnCysGluTrpSerAlaPheSerCysPheGln
LysAlaGlnLeuLysSerAlaAsnThrGlyAsnAsnGluArgIleIleAsnValSerIle
LysLysLeuLysArgLysProProSerThrAsnAlaGlyArgArgGlnLysHisArgLeu
ThrCysProSerCysAspSerTyrGluLysLysProProLysGluPheLeuGluArgPhe
LysSerLeuLeuGlnLysMetIleHisGlnHisLeuSerSerArgThrHisGlySerGlu
AspSer
SEQ ID NO: 80
5′ Cloning site
AAGCTT
SEQ ID NO: 81
3′ cloning site
TCTAGA
SEQ ID NO: 82
3′ cloning site with TAA stop codon
TAATCTAGA
SEQ ID NO: 83
3′ cloning site with TGA stop codon
TGATCTAGA
SEQ ID NO: 84
3′ cloning site with TAG stop codon
TAGTCTAGA
SEQ ID NO: 85
L-HDAg (L1.0 - gt1)
ATGGGGAGGAGCGAGTCAAAAAGGAACAGGGATGGGAGGGAAGGCATTCTGGAACAGTGGGT
CAACGGACGGAAAAAACTGGAGGATCTGGAAAGAGAGGCTAGGAAGATCAAGAAGAAAATCA
AGAAGTTAGAGGATGAAAATCCTTGGTTAGGAAACATCAAAGGAATCCTCGGAAAACGCGAC
AAGGATGGAGAAGGCGCCCCCCCTGCTAAAAGGGCACGCACAGACCAGATGGAGATCGATAG
CGGACCAGGAAAGCGACCACTGCGAGGAGGGTTTTCCGACAAAGAACGCCAGGATCACCGGA
GAAGGAAGGCCCTGGAGAACAAGCGGAAGCAGCTGGCCGCTGGAGGCAAACATCTGTCCAAG
GAGGAAGAGGAAGAGCTGAAGCGCCTGACCGAAGAGGACGAACGCCGAGAGCGGAGAACAGC
TGGACCAAGTGTGGGAGGAGTCAATCCCCTGGAGGGAGGATCACGGGGAGCACCTGGAGGAG
GATTTGTGCCAAACATGCTGTCTGTCCCTGAATCACCATTCAGCCGAACAGGCGAGGGGCTG
GATGTGAGGGGCAATCAGGGGTTCCCCTGGGACATCCTGTTTCCAGCAGATCCACCCTTCTC
CCCTCAGTCTTGCAGGCCACAGTAA
SEQ ID NO: 86
L1.0 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProTrpAspIleLeuPhe
ProAlaAspProProPheSerProGlnSerCysArgProGln
SEQ ID NO: 87
L-HDAg (L2.0 - gt2)
ATGGGACAGCCCGATAGCAGAAGACCTAGAAGAGGGAGGGAAGAAAGCCTGGGGAAATGGAT
TGACGCCCGAAGAAGGAAAGAAGAACTGGAGCGAGACCTGCGGAAAGTGAATAAGACAATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACAGACCAGATGGAGGTGGATAG
CGGCCCCCGGAAAAGAAAGCACCCTGGCGGGTTCACTGAACAGGAGCGGAGAGACCATAGGC
GCCGAAAGGCCCTGGAGAATAAGAAAAAGCAGCTGAGCTCCGGAGGCAAAGACCTGTCCAGG
GAGGAAGAGGAAGAGCTGCGGAGACTGACTGAAGAGGATGAAAGGCGAGAGCGACGAGTCGC
AGGACCTAGAGTGGGCGACGTGAACCCACTGGATGGAGGACCTAGGGGAGCACCAGGAGGAG
GATTCGTGCCTAGTATGCAGGGAATCCCCGAATCACCTTTTACCAGAAGGGGAGACGGCCTG
GATACACGCGGCACTCAGGAGTTCCCCTGGGTGAATCCTCAGCCACCACCTCCACGACTGCC
ACTGCTGGAATGCACCCCCCAGTAA
SEQ ID NO: 88
L2.0 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProTrpValAsnProGln
ProProProProArgLeuProLeuLeuGluCysThrProGln
SEQ ID NO: 89
S-HDAg (S1.0 - gt1)
ATGGGGCGGAGCGAGTCAAAGAGAAATAGGGACGGGAGAGAAGGCATCCTGGAGCAGTGGGT
CAATGGAAGAAAGAAACTGGAAGATCTGGAAAGAGAGGCAAGGAAGATCAAGAAGAAAATCA
AAAAATTAGAGGATGAAAATCCATGGTTAGGAAACATCAAAGGAATCCTCGGAAAAAGAGAC
AAGGATGGAGAAGGCGCCCCCCCTGCTAAAAGGGCACGCACAGACCAGATGGAGATCGATAG
TGGACCAGGAAAGCGACCTCTGCGAGGAGGGTTCTCAGACAAAGAACGCCAGGATCACCGGA
GAAGGAAGGCCCTGGAGAACAAGCGGAAGCAGCTGGCCGCTGGAGGCAAACATCTGAGCAAG
GAGGAAGAGGAAGAGCTGAAGCGCCTGACCGAAGAGGATGAACGCCGAGAGCGGAGAACAGC
TGGACCATCCGTGGGAGGAGTCAATCCCCTGGAGGGAGGATCTCGGGGAGCACCAGGAGGAG
GGTTCGTGCCTAACATGCTGAGCGTCCCAGAATCACCCTTTAGCCGAACTGGAGAGGGGCTG
GACGTGAGGGGCAATCAGGGGTTCCCCTAA
SEQ ID NO: 90
S1.0 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPhePro
SEQ ID NO: 91
S-HDAg (S2.0 - gt2)
ATGGGACAGCCTGATAGTAGGAGACCACGGAGAGGGAGAGAGGAGTCACTGGGAAAATGGAT
TGATGCTCGAAGACGGAAGGAGGAACTGGAGCGAGACCTGCGGAAAGTGAATAAGACTATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACTGACCAGATGGAGGTGGATAG
CGGCCCCCGGAAAAGAAAGCACCCTGGCGGGTTCACCGAACAGGAGCGGAGAGACCATAGGC
GCCGAAAGGCCCTGGAGAATAAGAAAAAGCAGCTGAGCTCCGGAGGCAAAGACCTGTCCCGC
GAGGAAGAGGAAGAGCTGCGGAGACTGACCGAAGAGGATGAAAGGCGAGAGCGACGAGTCGC
AGGACCAAGAGTGGGCGACGTGAACCCCCTGGATGGAGGACCAAGGGGAGCACCTGGAGGAG
GATTCGTGCCCAGTATGCAGGGAATCCCAGAATCACCCTTTACAAGAAGGGGAGACGGCCTG
GATACACGGGGCACTCAGGAGTTCCCTTAA
SEQ ID NO: 92
S2.0 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPhePro
SEQ ID NO: 93
L-HDAg-SHBcAg (L1.1 - gt1)
ATGGGGAGGAGCGAGTCAAAAAGGAACAGGGATGGGAGGGAAGGCATTCTGGAACAGTGGGT
CAACGGACGGAAAAAACTGGAGGATCTGGAAAGAGAGGCTAGGAAGATCAAGAAGAAAATCA
AGAAGTTAGAGGATGAAAATCCTTGGTTAGGAAACATCAAAGGAATCCTCGGAAAACGCGAC
AAGGATGGAGAAGGCGCCCCCCCTGCTAAAAGGGCACGCACAGACCAGATGGAGATCGATAG
CGGACCAGGAAAGCGACCACTGCGAGGAGGGTTTTCCGACAAAGAACGCCAGGATCACCGGA
GAAGGAAGGCCCTGGAGAACAAGCGGAAGCAGCTGGCCGCTGGAGGCAAACATCTGTCCAAG
GAGGAAGAGGAAGAGCTGAAGCGCCTGACCGAAGAGGACGAACGCCGAGAGCGGAGAACAGC
TGGACCAAGTGTGGGAGGAGTCAATCCCCTGGAGGGAGGATCACGGGGAGCACCTGGAGGAG
GATTTGTGCCAAACATGCTGTCTGTCCCTGAATCACCATTCAGCCGAACAGGCGAGGGGCTG
GATGTGAGGGGCAATCAGGGGTTCCCCTGGGACATCCTGTTTCCAGCAGATCCACCCTTCTC
CCCTCAGTCTTGCAGGCCACAGGGAAGCGGAGCTACTAACTTCAGCCTGCTGAAGCAGGCTG
GAGACGTGGAGGAGAACCCTGGACCTATGGATGTCAATGCCAGCAGAGCACTGGCCAACGTG
TACGACCTGCCCGACGATTTCTTTCCTCAGATCGACGATCTGGTGAGGGACGCCAAAGATGC
TCTGGAACCCTATTGGAAGGCAGAGACAATCAAAAAGCACGTGCTGATTGCCACTCATTTTG
TCGACCTGATTGAGGATTTCTGGCAGACCACACAGGGCATGTCTCAGATCGCTGACGCACTG
AGAGCCGTGATTCCTCCAACTACCACACCAGTCCCCGATGGATATCTGATTTCCCATAACGA
GGCCCAAGAATTACCACTCAATGACTTATTCGTCCTGCAGGAGGAAAGAATCGTGAACTTTC
AACCTGACTATCCAATTACTGCTAGAATTCACACCCACTTAAGAGTCTACACCAAGTTAAAC
GAGCAAGCCCTGGACAAGGCCAGGCGCCTGCTGTGGTGGCATTACAACTGTCTGCTGTGGGG
CGAGAGCAACGTGACCAATTATATCTCCCGACTGCGGACATGGCTGTCTACTCCAGAAAAAT
ACAGAGGGAAGGATGCTCCTACTATCGAGGCAATTACCCGACCAATTCAGGTCGCACAGGGA
AGCCGGAACCAGACCAAAGGAGTGCGGAAGCCAAGAGGCCTGGAGCCACGACGGAGAAAAGT
GAAGACTACCGTGGTCTATGGCAGGCGCCGATCTAAGAGTAGAGGGCGGAGAAGCTCCCCTA
GCCAGAGGGCAGGAAGCCCCATCCCCAGAAACAGAGAAAACCAGTCCAGAAGCAGCAGCCCA
AGAGAGTAA
SEQ ID NO: 94
L1.1 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProTrpAspIleLeuPhe
ProAlaAspProProPheSerProGlnSerCysArgProGlnGlySerGlyAlaThrAsn
PheSerLeuLeuLysGlnAlaGlyAspValGluGluAsnProGlyProMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyMetSerGlnIleAlaAspAlaLeuArgAlaValIleProProThrThrThrPro
ValProAspGlyTyrLeuIleSerHisAsnGluAlaGlnGluLeuProLeuAsnAspLeu
PheValLeuGlnGluGluArgIleValAsnPheGlnProAspTyrProIleThrAlaArg
IleHisThrHisLeuArgValTyrThrLysLeuAsnGluGlnAlaLeuAspLysAlaArg
ArgLeuLeuTrpTrpHisTyrAsnCysLeuLeuTrpGlyGluSerAsnValThrAsnTyr
IleSerArgLeuArgThrTrpLeuSerThrProGluLysTyrArgGlyLysAspAlaPro
ThrIleGluAlaIleThrArgProIleGlnValAlaGlnGlySerArgAsnGlnThrLys
GlyValArgLysProArgGlyLeuGluProArgArgArgLysValLysThrThrValVal
TyrGlyArgArgArgSerLysSerArgGlyArgArgSerSerProSerGlnArgAlaGly
SerProIleProArgAsnArgGluAsnGlnSerArgSerSerSerProArgGlu
SEQ ID NO: 95
L-HDAg-SHBcAg (L2.1 - gt2)
ATGGGACAGCCCGATAGCAGAAGACCTAGAAGAGGGAGGGAAGAAAGCCTGGGGAAATGGAT
TGACGCCCGAAGAAGGAAAGAAGAACTGGAGCGAGACCTGCGGAAAGTGAATAAGACAATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACAGACCAGATGGAGGTGGATAG
CGGCCCCCGGAAAAGAAAGCACCCTGGCGGGTTCACTGAACAGGAGCGGAGAGACCATAGGC
GCCGAAAGGCCCTGGAGAATAAGAAAAAGCAGCTGAGCTCCGGAGGCAAAGACCTGTCCAGG
GAGGAAGAGGAAGAGCTGCGGAGACTGACTGAAGAGGATGAAAGGCGAGAGCGACGAGTCGC
AGGACCTAGAGTGGGCGACGTGAACCCACTGGATGGAGGACCTAGGGGAGCACCAGGAGGAG
GATTCGTGCCTAGTATGCAGGGAATCCCCGAATCACCTTTTACCAGAAGGGGAGACGGCCTG
GATACACGCGGCACTCAGGAGTTCCCCTGGGTGAATCCTCAGCCACCACCTCCACGACTGCC
ACTGCTGGAATGCACCCCCCAGGGAAGCGGAGCTACTAACTTCAGCCTGCTGAAGCAGGCTG
GAGACGTGGAGGAGAACCCTGGACCTATGGATGTCAATGCCAGTAGAGCCCTGGCTAACGTG
TACGACCTGCCAGACGATTTCTTTCCCCAGATCGACGATCTGGTGAGGGACGCTAAGGATGC
ACTGGAACCATATTGGAAAGCCGAGACTATCAAAAAGCACGTGCTGATTGCTACCCATTTCG
TCGACCTGATTGAGGATTTTTGGCAGACCACACAGGGAATGAGTCAGATCGCAGACGCACTG
CGAGCTGTGATTCCACCTACTACCACACCTGTCCCAGATGGATACTTAATTTCCCATAACGA
GGCCCAAGAATTACCTTTAAATGACTTATTTGTCCTGCAGGAGGAAAGAATCGTGAACTTCC
AACCTGACTATCCTATTACCGCCAGAATTCACACTCACTTAAGAGTGTACACTAAACTCAAC
GAACAAGCCCTGGACAAGGCCCGCCGACTGCTGTGGTGGCATTACAACTGTCTGCTGTGGGG
CGAAAGCAACGTGACAAATTATATCTCCAGGCTGCGCACCTGGCTGTCTACACCTGAAAAGT
ACCGGGGCAAAGATGCACCCACAATCGAGGCCATTACTAGACCTATTCAGGTCGCCCAGGGG
TCTAGGAACCAGACCAAGGGGGTGAGAAAACCTAGGGGACTGGAGCCACGGAGAAGGAAGGT
GAAGACTACCGTGGTCTATGGACGCCGACGGTCAAAGAGCCGCGGCAGAAGGTCTAGTCCAA
GCCAGCGGGCAGGGTCACCAATCCCCAGAAACCGAGAGAATCAGTCCAGGTCATCATCACCA
AGAGAATAA
SEQ ID NO: 96
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProTrpValAsnProGln
ProProProProArgLeuProLeuLeuGluCysThrProGlnGlySerGlyAlaThrAsn
PheSerLeuLeuLysGlnAlaGlyAspValGluGluAsnProGlyProMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyMetSerGlnIleAlaAspAlaLeuArgAlaValIleProProThrThrThrPro
ValProAspGlyTyrLeuIleSerHisAsnGluAlaGlnGluLeuProLeuAsnAspLeu
PheValLeuGlnGluGluArgIleValAsnPheGlnProAspTyrProIleThrAlaArg
IleHisThrHisLeuArgValTyrThrLysLeuAsnGluGlnAlaLeuAspLysAlaArg
ArgLeuLeuTrpTrpHisTyrAsnCysLeuLeuTrpGlyGluSerAsnValThrAsnTyr
IleSerArgLeuArgThrTrpLeuSerThrProGluLysTyrArgGlyLysAspAlaPro
ThrIleGluAlaIleThrArgProIleGlnValAlaGlnGlySerArgAsnGlnThrLys
GlyValArgLysProArgGlyLeuGluProArgArgArgLysValLysThrThrValVal
TyrGlyArgArgArgSerLysSerArgGlyArgArgSerSerProSerGlnArgAlaGly
SerProIleProArgAsnArgGluAsnGlnSerArgSerSerSerProArgGlu
SEQ ID NO: 97
S-HDAg-SHBcAg (S1.1 - 01)
ATGGGGCGGAGCGAGTCAAAGAGAAATAGGGACGGGAGAGAAGGCATCCTGGAGCAGTGGGT
CAATGGAAGAAAGAAACTGGAAGATCTGGAAAGAGAGGCAAGGAAGATCAAGAAGAAAATCA
AAAAATTAGAGGATGAAAATCCATGGTTAGGAAACATCAAAGGAATCCTCGGAAAAAGAGAC
AAGGATGGAGAAGGCGCCCCCCCTGCTAAAAGGGCACGCACAGACCAGATGGAGATCGATAG
TGGACCAGGAAAGCGACCTCTGCGAGGAGGGTTCTCAGACAAAGAACGCCAGGATCACCGGA
GAAGGAAGGCCCTGGAGAACAAGCGGAAGCAGCTGGCCGCTGGAGGCAAACATCTGAGCAAG
GAGGAAGAGGAAGAGCTGAAGCGCCTGACCGAAGAGGATGAACGCCGAGAGCGGAGAACAGC
TGGACCATCCGTGGGAGGAGTCAATCCCCTGGAGGGAGGATCTCGGGGAGCACCAGGAGGAG
GGTTCGTGCCTAACATGCTGAGCGTCCCAGAATCACCCTTTAGCCGAACTGGAGAGGGGCTG
GACGTGAGGGGCAATCAGGGGTTCCCCGGAAGCGGAGCTACTAACTTCAGCCTGCTGAAGCA
GGCTGGAGACGTGGAGGAGAACCCTGGACCTATGGATGTCAATGCCTCCAGAGCACTGGCCA
ACGTGTACGATCTGCCAGACGATTTCTTTCCCCAGATCGACGATCTGGTGAGGGACGCCAAA
GATGCTCTGGAACCTTATTGGAAGGCAGAGACAATCAAAAAGCACGTGCTGATTGCCACTCA
TTTCGTCGACCTGATTGAGGATTTTTGGCAGACCACACAGGGCATGAGCCAGATCGCTGACG
CACTGAGAGCCGTGATTCCACCCACTACCACACCCGTCCCTGATGGATACTTAATTAGCCAT
AACGAGGCCCAAGAATTACCTTTAAATGACTTATTTGTCCTGCAGGAGGAAAGAATCGTGAA
CTTCCAACCAGACTATCCTATTACTGCCAGAATTCACACCCACTTAAGAGTCTACACCAAGT
TAAACGAGCAAGCCCTGGACAAGGCCAGGCGCCTGCTGTGGTGGCATTACAACTGCCTGCTG
TGGGGCGAGAGTAACGTGACTAATTATATCTCACGACTGCGGACATGGCTGAGCACTCCAGA
AAAATACCGGGGGAAGGATGCTCCTACTATCGAGGCAATTACCCGCCCAATTCAGGTCGCCC
AGGGCTCTCGGAACCAGACCAAAGGAGTGCGGAAGCCTAGAGGCCTGGAGCCACGACGGAGA
AAAGTGAAGACTACCGTGGTCTATGGCAGGCGCCGATCTAAGAGTAGAGGGCGGAGAAGCTC
CCCCAGTCAGAGGGCAGGGTCACCAATCCCAAGAAACAGAGAAAATCAGAGCAGGTCCTCCA
GCCCACGAGAATAA
SEQ ID NO: 98
S1.1 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProGlySerGlyAlaThr
AsnPheSerLeuLeuLysGlnAlaGlyAspValGluGluAsnProGlyProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyMetSerGlnIleAlaAspAlaLeuArgAlaValIleProProThrThrThr
ProValProAspGlyTyrLeuIleSerHisAsnGluAlaGlnGluLeuProLeuAsnAsp
LeuPheValLeuGlnGluGluArgIleValAsnPheGlnProAspTyrProIleThrAla
ArgIleHisThrHisLeuArgValTyrThrLysLeuAsnGluGlnAlaLeuAspLysAla
ArgArgLeuLeuTrpTrpHisTyrAsnCysLeuLeuTrpGlyGluSerAsnValThrAsn
TyrIleSerArgLeuArgThrTrpLeuSerThrProGluLysTyrArgGlyLysAspAla
ProThrIleGluAlaIleThrArgProIleGlnValAlaGlnGlySerArgAsnGlnThr
LysGlyValArgLysProArgGlyLeuGluProArgArgArgLysValLysThrThrVal
ValTyrGlyArgArgArgSerLysSerArgGlyArgArgSerSerProSerGlnArgAla
GlySerProIleProArgAsnArgGluAsnGlnSerArgSerSerSerProArgGlu
SEQ ID NO: 99
S-HDAg-SHBcAg (S2.1 - gt2)
ATGGGACAGCCTGATAGTAGGAGACCACGGAGAGGGAGAGAGGAGTCACTGGGAAAATGGAT
TGATGCTCGAAGACGGAAGGAGGAACTGGAGCGAGACCTGCGGAAAGTGAATAAGACTATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACTGACCAGATGGAGGTGGATAG
CGGCCCCCGGAAAAGAAAGCACCCTGGCGGGTTCACCGAACAGGAGCGGAGAGACCATAGGC
GCCGAAAGGCCCTGGAGAATAAGAAAAAGCAGCTGAGCTCCGGAGGCAAAGACCTGTCCCGC
GAGGAAGAGGAAGAGCTGCGGAGACTGACCGAAGAGGATGAAAGGCGAGAGCGACGAGTCGC
AGGACCAAGAGTGGGCGACGTGAACCCCCTGGATGGAGGACCAAGGGGAGCACCTGGAGGAG
GATTCGTGCCCAGTATGCAGGGAATCCCAGAATCACCCTTTACAAGAAGGGGAGACGGCCTG
GATACACGGGGCACTCAGGAGTTCCCTGGAAGCGGAGCTACTAACTTCAGCCTGCTGAAGCA
GGCTGGAGACGTGGAGGAGAACCCTGGACCTATGGATGTCAATGCCAGTCGCGCCCTGGCTA
ACGTGTACGACCTGCCAGACGATTTCTTTCCCCAGATCGACGATCTGGTGCGGGACGCTAAG
GATGCACTGGAACCATATTGGAAAGCCGAGACCATCAAAAAGCACGTGCTGATTGCTACACA
TTTCGTCGACCTGATTGAGGATTTTTGGCAGACCACACAGGGAATGAGTCAGATCGCAGACG
CCCTGAGAGCTGTGATTCCACCCACTACCACACCCGTCCCTGATGGATACTTAATTTCCCAT
AACGAGGCCCAAGAATTACCTTTAAATGACTTATTTGTCCTGCAGGAGGAAAGAATCGTGAA
CTTCCAACCAGACTATCCTATTACCGCCAGAATTCACACTCACTTAAGAGTCTACACTAAAC
TCAACGAGCAAGCCCTGGACAAGGCCCGCCGACTGCTGTGGTGGCATTACAACTGCCTGCTG
TGGGGCGAGAGCAACGTGACAAATTATATCTCCAGGCTGCGCACCTGGCTGTCTACACCTGA
AAAGTACAGGGGCAAAGATGCACCTACAATCGAGGCCATTACTAGACCAATTCAGGTCGCCC
AGGGGTCTAGGAACCAGACAAAGGGGGTGAGAAAACCTAGGGGACTGGAGCCACGGAGAAGG
AAGGTGAAGACTACCGTGGTCTATGGACGCCGACGGTCAAAGAGCCGCGGCAGAAGGTCTAG
TCCCAGCCAGCGGGCAGGCAGCCCAATCCCACGCAACAGAGAAAATCAGTCACGGTCCAGCA
GCCCCAGAGAATAA
SEQ ID NO: 100
S2.1 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProGlySerGlyAlaThr
AsnPheSerLeuLeuLysGlnAlaGlyAspValGluGluAsnProGlyProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyMetSerGlnIleAlaAspAlaLeuArgAlaValIleProProThrThrThr
ProValProAspGlyTyrLeuIleSerHisAsnGluAlaGlnGluLeuProLeuAsnAsp
LeuPheValLeuGlnGluGluArgIleValAsnPheGlnProAspTyrProIleThrAla
ArgIleHisThrHisLeuArgValTyrThrLysLeuAsnGluGlnAlaLeuAspLysAla
ArgArgLeuLeuTrpTrpHisTyrAsnCysLeuLeuTrpGlyGluSerAsnValThrAsn
TyrIleSerArgLeuArgThrTrpLeuSerThrProGluLysTyrArgGlyLysAspAla
ProThrIleGluAlaIleThrArgProIleGlnValAlaGlnGlySerArgAsnGlnThr
LysGlyValArgLysProArgGlyLeuGluProArgArgArgLysValLysThrThrVal
ValTyrGlyArgArgArgSerLysSerArgGlyArgArgSerSerProSerGlnArgAla
GlySerProIleProArgAsnArgGluAsnGlnSerArgSerSerSerProArgGlu
SEQ ID NO: 101
L-HDAg-frag-SHBcAg (L1.2 - gt1)
ATGGGGAGGAGTGAGTCAAAACGAAATAGAGATGGCAGGGAAGGGATTCTGGAGCAGTGGGT
CAATGGACGGAAAAAACTGGAGGACCTGGAAAGAGAGGCCAGGAAGATCAAGAAGAAAATCA
AAAAATTAGAGGATGAAAATCCTTGGTTAGGAAACATCAAAGGAATCCTCGGAAAACGAGAC
AAGGATGGAGAAGGAGCACCACCTGCTAAAAGGGCACGCACAGACCAGATGGAGATCGATTC
CGGACCAGGCAAGCGACCACTGCGAGGAGGGTTTTCTGACAAAGAACGCCAGGATCACCGGA
GAAGGAAGGCCCTGGAGAACAAGCGGAAGCAGCTGGCCGCTGGAGGCAAACATCTGAGCAAG
GAGGAAGAGGAAGAGCTGAAGCGCCTGACCGAAGAGGACGAACGCCGAGAGCGGAGAACAGC
TGGACCATCAGTGGGAGGAGTCAATCCCCTGGAGGGAGGAAGCCGGGGAGCACCTGGAGGAG
GGTTTGTGCCAAACATGCTGTCCGTCCCTGAATCACCATTCAGCCGAACCGGGGAGGGACTG
GATGTGAGGGGCAATCAGGGGTTCCCCTGGGACATCCTGTTTCCTGCTGATCCACCCTTCTC
CCCTCAGTCTTGCAGGCCACAGGGAAGCGGAGCTACTAACTTCAGCCTGCTGAAGCAGGCTG
GAGACGTGGAGGAGAACCCTGGACCTATGGATGTCAATGCCAGCAGAGCACTGGCCAACGTG
TACGACCTGCCCGACGATTTCTTTCCTCAGATCGACGATCTGGTGAGGGACGCCAAAGATGC
TCTGGAACCCTATTGGAAGGCAGAGACAATCAAAAAGCACGTGCTGATTGCCACTCATTTTG
TCGACCTGATTGAGGATTTCTGGCAGACCACACAGGGAGGAAGCGGAGCTACTAACTTCAGC
CTGCTGAAGCAGGCTGGAGACGTGGAGGAGAACCCTGGACCTATGAGTCAGATCGCTGATGC
ACTGAGAGCTGTGATTCCTCCAACTACCACACCTGTCCCAGATGGATACTTAATTTCCCATA
ACGAGGCTCAAGAATTACCACTAAATGACTTATTCGTCCTGCAGGAGGAAAGAATCGTGAAC
TTTCAACCTGACTATCCAATTACTGCTAGAATTCACACACATCTGCGCGTCTATGGAAGCGG
AGCTACTAACTTCAGCCTGCTGAAGCAGGCTGGAGACGTGGAGGAGAACCCTGGACCTACCA
AGTTAAATGAACAAGCCCTGGACAAGGCCAGGCGCCTGCTGTGGTGGCATTACAATTGTCTG
CTGTGGGGCGAGTCCAACGTGACCAATTATATCTCTCGACTGCGGACATGGCTGAGTACTCC
AGAAAAATACAGAGGGAAGGACGCTCCTACTATCGAGGCAATTACCAGGCCAATTCAGGTGG
CCCAGGGCGGAAGCGGAGCTACTAACTTCAGCCTGCTGAAGCAGGCTGGAGACGTGGAGGAG
AACCCTGGACCTTCCAGAAATCAGACTAAAGGAGTCCGGAAGCCAAGAGGCCTGGAGCCACG
ACGGAGAAAAGTGAAGACTACCGTGGTCTATGGCAGGCGCCGATCTAAGAGTCGCGGGCGGA
GAAGCTCCCCTTCTCAGCGGGCAGGGTCACCTATCCCACGGAACAGAGAGAACCAGAGCAGA
AGCAGCAGTCCAAGAGAGTAA
SEQ ID NO: 102
L1.2 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProTrpAspIleLeuPhe
ProAlaAspProProPheSerProGlnSerCysArgProGlnGlySerGlyAlaThrAsn
PheSerLeuLeuLysGlnAlaGlyAspValGluGluAsnProGlyProMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyGlySerGlyAlaThrAsnPheSerLeuLeuLysGlnAlaGlyAspValGluGlu
AsnProGlyProMetSerGlnIleAlaAspAlaLeuArgAlaValIleProProThrThr
ThrProValProAspGlyTyrLeuIleSerHisAsnGluAlaGlnGluLeuProLeuAsn
AspLeuPheValLeuGlnGluGluArgIleValAsnPheGlnProAspTyrProIleThr
AlaArgIleHisThrHisLeuArgValTyrGlySerGlyAlaThrAsnPheSerLeuLeu
LysGlnAlaGlyAspValGluGluAsnProGlyProThrLysLeuAsnGluGlnAlaLeu
AspLysAlaArgArgLeuLeuTrpTrpHisTyrAsnCysLeuLeuTrpGlyGluSerAsn
ValThrAsnTyrIleSerArgLeuArgThrTrpLeuSerThrProGluLysTyrArgGly
LysAspAlaProThrIleGluAlaIleThrArgProIleGlnValAlaGlnGlyGlySer
GlyAlaThrAsnPheSerLeuLeuLysGlnAlaGlyAspValGluGluAsnProGlyPro
SerArgAsnGlnThrLysGlyValArgLysProArgGlyLeuGluProArgArgArgLys
ValLysThrThrValValTyrGlyArgArgArgSerLysSerArgGlyArgArgSerSer
ProSerGlnArgAlaGlySerProIleProArgAsnArgGluAsnGlnSerArgSerSer
SerProArgGlu
SEQ ID NO: 103
L-HDAg-frag-SHBcAg (L2.2 - gt2)
ATGGGACAGCCTGATTCACGGAGACCACGGAGGGGCAGAGAGGAGTCACTGGGGAAATGGAT
TGATGCTCGCAGAAGAAAAGAAGAACTGGAGCGAGACCTGCGGAAAGTGAATAAGACCATCA
AAAGACTGGAGGAAGATAACCCCTGGCTGGGCAATATTCGCGGCATCATTGGGCGAAAAGAC
AAGGATGGAGAAGGAGCTCCACCTGCAAAGCGAGCACGAACCGACCAGATGGAGGTGGATTC
CGGCCCCCGGAAAAGAAAGCACCCTGGCGGGTTTACAGAACAGGAGCGGAGAGACCATAGGC
GCCGAAAGGCCCTGGAGAATAAGAAAAAGCAGCTGAGCTCCGGAGGCAAAGACCTGTCTAGG
GAGGAAGAGGAAGAGCTGCGGAGACTGACAGAAGAGGATGAAAGGCGAGAGCGACGAGTCGC
AGGACCTCGAGTGGGCGACGTGAACCCACTGGATGGAGGACCTCGAGGAGCACCAGGAGGAG
GATTCGTGCCTTCAATGCAGGGAATCCCCGAAAGCCCTTTTACTAGAAGGGGCGACGGGCTG
GATACACGAGGAACTCAGGAGTTCCCCTGGGTGAATCCTCAGCCACCACCTCCACGACTGCC
ACTGCTGGAATGCACCCCCCAGGGAAGCGGAGCTACTAACTTCAGCCTGCTGAAGCAGGCTG
GAGACGTGGAGGAGAACCCTGGACCTATGGATGTCAATGCCTCCAGAGCCCTGGCTAACGTG
TACGACCTGCCAGACGATTTCTTTCCCCAGATCGACGATCTGGTGAGGGACGCTAAGGATGC
ACTGGAACCATATTGGAAAGCCGAGACTATCAAAAAGCACGTGCTGATTGCTACCCATTTCG
TCGACCTGATTGAGGATTTTTGGCAGACCACACAGGGCGGAAGCGGAGCTACTAACTTCAGC
CTGCTGAAGCAGGCTGGAGACGTGGAGGAGAACCCTGGACCTATGTCACAGATCGCAGATGC
CCTGCGCGCTGTGATTCCCCCTACTACCACACCCGTCCCTGATGGATACTTAATTTCCCATA
ACGAGGCTCAAGAATTACCTTTAAATGACTTATTTGTCCTGCAGGAGGAAAGAATCGTGAAC
TTCCAACCTGACTATCCTATTACCGCCAGAATTCACACCCATCTGCGGGTCTATGGAAGCGG
AGCTACTAACTTCAGCCTGCTGAAGCAGGCTGGAGACGTGGAGGAGAACCCTGGACCTACCA
AACTCAATGAACAAGCCCTGGACAAGGCCCGCCGACTGCTGTGGTGGCATTACAATTGTCTG
CTGTGGGGCGAGTCCAACGTGACCAATTATATCTCTAGGCTGCGCACCTGGCTGAGTACACC
TGAAAAGTACAGAGGCAAAGACGCACCCACAATCGAGGCCATTACTAGGCCTATTCAGGTCG
CTCAGGGAGGAAGCGGAGCTACTAACTTCAGCCTGCTGAAGCAGGCTGGAGACGTGGAGGAG
AACCCTGGACCTAGTCGGAATCAGACCAAGGGGGTCAGAAAACCTAGGGGACTGGAGCCACG
GAGAAGGAAGGTGAAGACTACCGTGGTCTATGGACGCCGACGGTCAAAAAGCAGAGGCAGAA
GGTCTAGTCCAAGCCAGAGGGCAGGCAGCCCTATCCCACGGAACAGAGAAAACCAGAGCAGG
TCCAGCAGTCCAAGAGAATAA
SEQ ID NO: 104
L2.2 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProTrpValAsnProGln
ProProProProArgLeuProLeuLeuGluCysThrProGlnGlySerGlyAlaThrAsn
PheSerLeuLeuLysGlnAlaGlyAspValGluGluAsnProGlyProMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyGlySerGlyAlaThrAsnPheSerLeuLeuLysGlnAlaGlyAspValGluGlu
AsnProGlyProMetSerGlnIleAlaAspAlaLeuArgAlaValIleProProThrThr
ThrProValProAspGlyTyrLeuIleSerHisAsnGluAlaGlnGluLeuProLeuAsn
AspLeuPheValLeuGlnGluGluArgIleValAsnPheGlnProAspTyrProIleThr
AlaArgIleHisThrHisLeuArgValTyrGlySerGlyAlaThrAsnPheSerLeuLeu
LysGlnAlaGlyAspValGluGluAsnProGlyProThrLysLeuAsnGluGlnAlaLeu
AspLysAlaArgArgLeuLeuTrpTrpHisTyrAsnCysLeuLeuTrpGlyGluSerAsn
ValThrAsnTyrIleSerArgLeuArgThrTrpLeuSerThrProGluLysTyrArgGly
LysAspAlaProThrIleGluAlaIleThrArgProIleGlnValAlaGlnGlyGlySer
GlyAlaThrAsnPheSerLeuLeuLysGlnAlaGlyAspValGluGluAsnProGlyPro
SerArgAsnGlnThrLysGlyValArgLysProArgGlyLeuGluProArgArgArgLys
ValLysThrThrValValTyrGlyArgArgArgSerLysSerArgGlyArgArgSerSer
ProSerGlnArgAlaGlySerProIleProArgAsnArgGluAsnGlnSerArgSerSer
SerProArgGlu
SEQ ID NO: 105
S-HDAg-frag-SHBcAg (S1.2 - gt1)
ATGGGACGGAGTGAGTCAAAGAGAAATAGAGACGGACGGGAGGGCATCCTGGAGCAGTGGGT
CAATGGACGGAAGAAACTGGAAGACCTGGAAAGAGAGGCTAGGAAGATCAAGAAGAAAATCA
AAAAATTAGAGGATGAAAATCCATGGTTAGGAAACATCAAAGGAATCCTCGGAAAAAGAGAC
AAGGATGGCGAAGGGGCCCCCCCTGCTAAAAGGGCACGCACAGACCAGATGGAGATCGATTC
AGGACCAGGCAAGCGACCTCTGCGAGGAGGGTTTAGCGACAAAGAACGCCAGGATCACCGGA
GAAGGAAGGCCCTGGAGAACAAGCGGAAGCAGCTGGCCGCTGGAGGCAAACATCTGTCCAAG
GAGGAAGAGGAAGAGCTGAAGCGCCTGACCGAAGAGGATGAACGCCGAGAGCGGAGAACAGC
TGGACCATCTGTGGGAGGAGTCAATCCCCTGGAGGGAGGAAGTCGGGGAGCACCAGGAGGAG
GGTTCGTGCCTAACATGCTGTCTGTCCCAGAATCACCCTTTAGCCGAACTGGGGAGGGACTG
GACGTGAGGGGCAATCAGGGGTTCCCCGGAAGCGGAGCTACTAACTTCAGCCTGCTGAAGCA
GGCTGGAGACGTGGAGGAGAACCCTGGACCTATGGATGTCAATGCCAGCAGAGCACTGGCCA
ACGTGTACGATCTGCCAGACGATTTCTTTCCCCAGATCGACGATCTGGTGAGGGACGCCAAA
GATGCTCTGGAACCTTATTGGAAGGCAGAGACAATCAAAAAGCACGTGCTGATTGCCACTCA
TTTCGTCGACCTGATTGAGGATTTTTGGCAGACCACACAGGGAGGAAGCGGAGCTACTAACT
TCAGCCTGCTGAAGCAGGCTGGAGACGTGGAGGAGAACCCTGGACCTATGTCCCAGATCGCT
GATGCACTGAGAGCTGTGATTCCACCCACTACCACACCAGTCCCCGACGGATACTTAATTTC
CCATAACGAGGCTCAAGAATTACCTTTAAATGACTTATTTGTCCTGCAGGAGGAAAGAATCG
TGAACTTCCAACCAGACTATCCTATTACTGCTAGAATTCACACACATCTGCGCGTCTATGGA
AGCGGAGCTACTAACTTCAGCCTGCTGAAGCAGGCTGGAGACGTGGAGGAGAACCCTGGACC
TACCAAGTTAAATGAACAAGCCCTGGACAAGGCCAGGCGCCTGCTGTGGTGGCATTACAATT
GCCTGCTGTGGGGCGAGTCAAACGTGACCAATTATATCAGCCGACTGCGGACATGGCTGTCC
ACTCCAGAAAAATACAGAGGGAAGGACGCTCCTACTATCGAGGCAATTACCAGGCCAATTCA
GGTGGCCCAGGGGGGAAGCGGAGCTACTAACTTCAGCCTGCTGAAGCAGGCTGGAGACGTGG
AGGAGAACCCTGGACCTAGTCGGAATCAGACAAAAGGAGTCCGGAAGCCTAGAGGCCTGGAG
CCACGACGGAGAAAAGTGAAGACTACCGTGGTCTATGGCAGGCGCCGATCTAAGAGTCGCGG
GCGGAGAAGCTCCCCCAGCCAGCGGGCAGGCAGCCCAATCCCCCGCAACAGAGAAAACCAGT
CCCGCTCCTCCTCACCACGAGAGTAA
SEQ ID NO: 106
S1.2 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProGlySerGlyAlaThr
AsnPheSerLeuLeuLysGlnAlaGlyAspValGluGluAsnProGlyProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyGlySerGlyAlaThrAsnPheSerLeuLeuLysGlnAlaGlyAspValGlu
GluAsnProGlyProMetSerGlnIleAlaAspAlaLeuArgAlaValIleProProThr
ThrThrProValProAspGlyTyrLeuIleSerHisAsnGluAlaGlnGluLeuProLeu
AsnAspLeuPheValLeuGlnGluGluArgIleValAsnPheGlnProAspTyrProIle
ThrAlaArgIleHisThrHisLeuArgValTyrGlySerGlyAlaThrAsnPheSerLeu
LeuLysGlnAlaGlyAspValGluGluAsnProGlyProThrLysLeuAsnGluGlnAla
LeuAspLysAlaArgArgLeuLeuTrpTrpHisTyrAsnCysLeuLeuTrpGlyGluSer
AsnValThrAsnTyrIleSerArgLeuArgThrTrpLeuSerThrProGluLysTyrArg
GlyLysAspAlaProThrIleGluAlaIleThrArgProIleGlnValAlaGlnGlyGly
SerGlyAlaThrAsnPheSerLeuLeuLysGlnAlaGlyAspValGluGluAsnProGly
ProSerArgAsnGlnThrLysGlyValArgLysProArgGlyLeuGluProArgArgArg
LysValLysThrThrValValTyrGlyArgArgArgSerLysSerArgGlyArgArgSer
SerProSerGlnArgAlaGlySerProIleProArgAsnArgGluAsnGlnSerArgSer
SerSerProArgGlu
SEQ ID NO: 107
S-HDAg-frag-SHBcAg (S2.2 - gt2)
ATGGGGCAGCCTGATTCACGGAGACCACGGAGAGGAAGAGAGGAGAGCCTGGGGAAATGGAT
TGACGCACGGAGACGGAAAGAGGAGCTGGAGCGAGACCTGCGGAAAGTGAATAAGACAATCA
AAAGACTGGAGGAAGATAACCCCTGGCTGGGCAATATTCGCGGCATCATTGGGCGAAAAGAC
AAGGATGGAGAAGGAGCTCCACCTGCAAAGCGAGCACGAACAGACCAGATGGAGGTGGATTC
CGGCCCCCGGAAAAGAAAGCACCCTGGCGGGTTTACTGAACAGGAGCGGAGAGACCATAGGC
GCCGAAAGGCCCTGGAGAATAAGAAAAAGCAGCTGAGCTCCGGAGGCAAAGACCTGTCTCGC
GAGGAAGAGGAAGAGCTGCGGAGACTGACCGAAGAGGATGAAAGGCGAGAGCGACGAGTGGC
AGGACCACGAGTGGGCGACGTGAACCCCCTGGATGGAGGACCACGAGGAGCACCTGGAGGAG
GATTCGTGCCCTCAATGCAGGGAATCCCAGAAAGCCCCTTTACCAGAAGGGGCGACGGGCTG
GATACACGAGGAACTCAGGAGTTCCCTGGAAGCGGAGCTACTAACTTCAGCCTGCTGAAGCA
GGCTGGAGACGTGGAGGAGAACCCTGGACCTATGGATGTCAATGCCTCCCGCGCCCTGGCTA
ACGTGTACGACCTGCCAGACGATTTCTTTCCCCAGATCGACGATCTGGTGCGGGACGCTAAG
GATGCACTGGAACCATATTGGAAAGCCGAGACCATCAAAAAGCACGTGCTGATTGCTACACA
TTTCGTCGACCTGATTGAGGATTTTTGGCAGACCACACAGGGCGGAAGCGGAGCTACTAACT
TCAGCCTGCTGAAGCAGGCTGGAGACGTGGAGGAGAACCCTGGACCTATGTCACAGATCGCA
GATGCCCTGAGAGCTGTGATTCCACCCACTACCACACCAGTCCCCGATGGATACTTAATTTC
CCATAACGAGGCTCAAGAATTACCTTTAAATGACTTATTTGTCCTGCAGGAGGAAAGAATCG
TGAACTTCCAACCAGACTATCCTATTACCGCCAGAATTCACACCCATCTGCGGGTCTATGGA
AGCGGAGCTACTAACTTCAGCCTGCTGAAGCAGGCTGGAGACGTGGAGGAGAACCCTGGACC
TACCAAACTCAATGAGCAAGCCCTGGACAAGGCCCGCCGACTGCTGTGGTGGCATTACAATT
GCCTGCTGTGGGGCGAGTCCAACGTGACCAATTATATCTCTAGGCTGCGCACCTGGCTGAGT
ACACCTGAAAAGTACAGAGGCAAAGACGCACCTACAATCGAGGCCATTACTAGGCCAATTCA
GGTCGCTCAGGGAGGAAGCGGAGCTACTAACTTCAGCCTGCTGAAGCAGGCTGGAGACGTGG
AGGAGAACCCTGGACCTAGTAGGAATCAGACCAAGGGGGTCAGAAAACCTAGGGGACTGGAG
CCACGGAGAAGGAAGGTGAAGACTACCGTGGTCTATGGACGCCGACGGTCAAAAAGCAGAGG
CAGAAGGTCTAGTCCCAGCCAGAGGGCAGGGTCACCTATCCCCAGAAACAGAGAAAACCAGT
CAAGGAGTTCATCCCCACGCGAGTAA
SEQ ID NO: 108
S2.2 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProGlySerGlyAlaThr
AsnPheSerLeuLeuLysGlnAlaGlyAspValGluGluAsnProGlyProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyGlySerGlyAlaThrAsnPheSerLeuLeuLysGlnAlaGlyAspValGlu
GluAsnProGlyProMetSerGlnIleAlaAspAlaLeuArgAlaValIleProProThr
ThrThrProValProAspGlyTyrLeuIleSerHisAsnGluAlaGlnGluLeuProLeu
AsnAspLeuPheValLeuGlnGluGluArgIleValAsnPheGlnProAspTyrProIle
ThrAlaArgIleHisThrHisLeuArgValTyrGlySerGlyAlaThrAsnPheSerLeu
LeuLysGlnAlaGlyAspValGluGluAsnProGlyProThrLysLeuAsnGluGlnAla
LeuAspLysAlaArgArgLeuLeuTrpTrpHisTyrAsnCysLeuLeuTrpGlyGluSer
AsnValThrAsnTyrIleSerArgLeuArgThrTrpLeuSerThrProGluLysTyrArg
GlyLysAspAlaProThrIleGluAlaIleThrArgProIleGlnValAlaGlnGlyGly
SerGlyAlaThrAsnPheSerLeuLeuLysGlnAlaGlyAspValGluGluAsnProGly
ProSerArgAsnGlnThrLysGlyValArgLysProArgGlyLeuGluProArgArgArg
LysValLysThrThrValValTyrGlyArgArgArgSerLysSerArgGlyArgArgSer
SerProSerGlnArgAlaGlySerProIleProArgAsnArgGluAsnGlnSerArgSer
SerSerProArgGlu
SEQ ID NO: 109
L-HDAg-fusion-SHBcAg (L1.3 - gt1)
ATGGGGAGGAGCGAGTCAAAAAGGAACAGGGATGGGAGGGAAGGCATTCTGGAACAGTGGGT
CAACGGACGGAAAAAACTGGAGGATCTGGAAAGAGAGGCTAGGAAGATCAAGAAGAAAATCA
AGAAGTTAGAGGATGAAAATCCTTGGTTAGGAAACATCAAAGGAATCCTCGGAAAACGCGAC
AAGGATGGAGAAGGCGCCCCCCCTGCTAAAAGGGCACGCACAGACCAGATGGAGATCGATAG
CGGACCAGGAAAGCGACCACTGCGAGGAGGGTTTTCCGACAAAGAACGCCAGGATCACCGGA
GAAGGAAGGCCCTGGAGAACAAGCGGAAGCAGCTGGCCGCTGGAGGCAAACATCTGTCCAAG
GAGGAAGAGGAAGAGCTGAAGCGCCTGACCGAAGAGGACGAACGCCGAGAGCGGAGAACAGC
TGGACCAAGTGTGGGAGGAGTCAATCCCCTGGAGGGAGGATCACGGGGAGCACCTGGAGGAG
GATTTGTGCCAAACATGCTGTCTGTCCCTGAATCACCATTCAGCCGAACAGGCGAGGGGCTG
GATGTGAGGGGCAATCAGGGGTTCCCCTGGGACATCCTGTTTCCAGCAGATCCACCCTTCTC
CCCTCAGTCTTGCAGGCCACAGATGGATGTCAATGCCAGCAGAGCACTGGCCAACGTGTACG
ACCTGCCCGACGATTTCTTTCCTCAGATCGACGATCTGGTGAGGGACGCCAAAGATGCTCTG
GAACCCTATTGGAAGGCAGAGACAATCAAAAAGCACGTGCTGATTGCCACTCATTTTGTCGA
CCTGATTGAGGATTTCTGGCAGACCACACAGGGCATGTCTCAGATCGCTGACGCACTGAGAG
CCGTGATTCCTCCAACTACCACACCAGTCCCCGATGGATATCTGATTTCCCATAACGAGGCC
CAAGAATTACCACTCAATGACTTATTCGTCCTGCAGGAGGAAAGAATCGTGAACTTTCAACC
TGACTATCCAATTACTGCTAGAATTCACACCCACTTAAGAGTCTACACCAAGTTAAACGAGC
AAGCCCTGGACAAGGCCAGGCGCCTGCTGTGGTGGCATTACAACTGTCTGCTGTGGGGCGAG
AGCAACGTGACCAATTATATCTCCCGACTGCGGACATGGCTGTCTACTCCAGAAAAATACAG
AGGGAAGGATGCTCCTACTATCGAGGCAATTACCCGACCAATTCAGGTCGCACAGGGAAGCC
GGAACCAGACCAAAGGAGTGCGGAAGCCAAGAGGCCTGGAGCCACGACGGAGAAAAGTGAAG
ACTACCGTGGTCTATGGCAGGCGCCGATCTAAGAGTAGAGGGCGGAGAAGCTCCCCTAGCCA
GAGGGCAGGAAGCCCCATCCCCAGAAACAGAGAAAACCAGTCCAGAAGCAGCAGCCCAAGAG
AGTAA
SEQ ID NO: 110
L1.3 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProTrpAspIleLeuPhe
ProAlaAspProProPheSerProGlnSerCysArgProGlnMetAspValAsnAlaSer
ArgAlaLeuAlaAsnValTyrAspLeuProAspAspPhePheProGlnIleAspAspLeu
ValArgAspAlaLysAspAlaLeuGluProTyrTrpLysAlaGluThrIleLysLysHis
ValLeuIleAlaThrHisPheValAspLeuIleGluAspPheTrpGlnThrThrGlnGly
MetSerGlnIleAlaAspAlaLeuArgAlaValIleProProThrThrThrProValPro
AspGlyTyrLeuIleSerHisAsnGluAlaGlnGluLeuProLeuAsnAspLeuPheVal
LeuGlnGluGluArgIleValAsnPheGlnProAspTyrProIleThrAlaArgIleHis
ThrHisLeuArgValTyrThrLysLeuAsnGluGlnAlaLeuAspLysAlaArgArgLeu
LeuTrpTrpHisTyrAsnCysLeuLeuTrpGlyGluSerAsnValThrAsnTyrIleSer
ArgLeuArgThrTrpLeuSerThrProGluLysTyrArgGlyLysAspAlaProThrIle
GluAlaIleThrArgProIleGlnValAlaGlnGlySerArgAsnGlnThrLysGlyVal
ArgLysProArgGlyLeuGluProArgArgArgLysValLysThrThrValValTyrGly
ArgArgArgSerLysSerArgGlyArgArgSerSerProSerGlnArgAlaGlySerPro
IleProArgAsnArgGluAsnGlnSerArgSerSerSerProArgGlu
SEQ ID NO: 111
L-HDAg-fusion-SHBcAg (L2.3 - gt2)
ATGGGACAGCCCGATAGCAGAAGACCTAGAAGAGGGAGGGAAGAAAGCCTGGGGAAATGGAT
TGACGCCCGAAGAAGGAAAGAAGAACTGGAGCGAGACCTGCGGAAAGTGAATAAGACAATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACAGACCAGATGGAGGTGGATAG
CGGCCCCCGGAAAAGAAAGCACCCTGGCGGGTTCACTGAACAGGAGCGGAGAGACCATAGGC
GCCGAAAGGCCCTGGAGAATAAGAAAAAGCAGCTGAGCTCCGGAGGCAAAGACCTGTCCAGG
GAGGAAGAGGAAGAGCTGCGGAGACTGACTGAAGAGGATGAAAGGCGAGAGCGACGAGTCGC
AGGACCTAGAGTGGGCGACGTGAACCCACTGGATGGAGGACCTAGGGGAGCACCAGGAGGAG
GATTCGTGCCTAGTATGCAGGGAATCCCCGAATCACCTTTTACCAGAAGGGGAGACGGCCTG
GATACACGCGGCACTCAGGAGTTCCCCTGGGTGAATCCTCAGCCACCACCTCCACGACTGCC
ACTGCTGGAATGCACCCCCCAGATGGATGTCAATGCCAGTAGAGCCCTGGCTAACGTGTACG
ACCTGCCAGACGATTTCTTTCCCCAGATCGACGATCTGGTGAGGGACGCTAAGGATGCACTG
GAACCATATTGGAAAGCCGAGACTATCAAAAAGCACGTGCTGATTGCTACCCATTTCGTCGA
CCTGATTGAGGATTTTTGGCAGACCACACAGGGAATGAGTCAGATCGCAGACGCACTGCGAG
CTGTGATTCCACCTACTACCACACCTGTCCCAGATGGATACTTAATTTCCCATAACGAGGCC
CAAGAATTACCTTTAAATGACTTATTTGTCCTGCAGGAGGAAAGAATCGTGAACTTCCAACC
TGACTATCCTATTACCGCCAGAATTCACACTCACTTAAGAGTGTACACTAAACTCAACGAAC
AAGCCCTGGACAAGGCCCGCCGACTGCTGTGGTGGCATTACAACTGTCTGCTGTGGGGCGAA
AGCAACGTGACAAATTATATCTCCAGGCTGCGCACCTGGCTGTCTACACCTGAAAAGTACCG
GGGCAAAGATGCACCCACAATCGAGGCCATTACTAGACCTATTCAGGTCGCCCAGGGGTCTA
GGAACCAGACCAAGGGGGTGAGAAAACCTAGGGGACTGGAGCCACGGAGAAGGAAGGTGAAG
ACTACCGTGGTCTATGGACGCCGACGGTCAAAGAGCCGCGGCAGAAGGTCTAGTCCAAGCCA
GCGGGCAGGGTCACCAATCCCCAGAAACCGAGAGAATCAGTCCAGGTCATCATCACCAAGAG
AATAA
SEQ ID NO: 112
L2.3 protien
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProTrpValAsnProGln
ProProProProArgLeuProLeuLeuGluCysThrProGlnMetAspValAsnAlaSer
ArgAlaLeuAlaAsnValTyrAspLeuProAspAspPhePheProGlnIleAspAspLeu
ValArgAspAlaLysAspAlaLeuGluProTyrTrpLysAlaGluThrIleLysLysHis
ValLeuIleAlaThrHisPheValAspLeuIleGluAspPheTrpGlnThrThrGlnGly
MetSerGlnIleAlaAspAlaLeuArgAlaValIleProProThrThrThrProValPro
AspGlyTyrLeuIleSerHisAsnGluAlaGlnGluLeuProLeuAsnAspLeuPheVal
LeuGlnGluGluArgIleValAsnPheGlnProAspTyrProIleThrAlaArgIleHis
ThrHisLeuArgValTyrThrLysLeuAsnGluGlnAlaLeuAspLysAlaArgArgLeu
LeuTrpTrpHisTyrAsnCysLeuLeuTrpGlyGluSerAsnValThrAsnTyrIleSer
ArgLeuArgThrTrpLeuSerThrProGluLysTyrArgGlyLysAspAlaProThrIle
GluAlaIleThrArgProIleGlnValAlaGlnGlySerArgAsnGlnThrLysGlyVal
ArgLysProArgGlyLeuGluProArgArgArgLysValLysThrThrValValTyrGly
ArgArgArgSerLysSerArgGlyArgArgSerSerProSerGlnArgAlaGlySerPro
IleProArgAsnArgGluAsnGlnSerArgSerSerSerProArgGlu
SEQ ID NO: 113
S-HDAg-fusion-SHBcAg (S1.3 - gt1)
ATGGGGCGGAGCGAGTCAAAGAGAAATAGGGACGGGAGAGAAGGCATCCTGGAGCAGTGGGT
CAATGGAAGAAAGAAACTGGAAGATCTGGAAAGAGAGGCAAGGAAGATCAAGAAGAAAATCA
AAAAATTAGAGGATGAAAATCCATGGTTAGGAAACATCAAAGGAATCCTCGGAAAAAGAGAC
AAGGATGGAGAAGGCGCCCCCCCTGCTAAAAGGGCACGCACAGACCAGATGGAGATCGATAG
TGGACCAGGAAAGCGACCTCTGCGAGGAGGGTTCTCAGACAAAGAACGCCAGGATCACCGGA
GAAGGAAGGCCCTGGAGAACAAGCGGAAGCAGCTGGCCGCTGGAGGCAAACATCTGAGCAAG
GAGGAAGAGGAAGAGCTGAAGCGCCTGACCGAAGAGGATGAACGCCGAGAGCGGAGAACAGC
TGGACCATCCGTGGGAGGAGTCAATCCCCTGGAGGGAGGATCTCGGGGAGCACCAGGAGGAG
GGTTCGTGCCTAACATGCTGAGCGTCCCAGAATCACCCTTTAGCCGAACTGGAGAGGGGCTG
GACGTGAGGGGCAATCAGGGGTTCCCCATGGATGTCAATGCCTCCAGAGCACTGGCCAACGT
GTACGATCTGCCAGACGATTTCTTTCCCCAGATCGACGATCTGGTGAGGGACGCCAAAGATG
CTCTGGAACCTTATTGGAAGGCAGAGACAATCAAAAAGCACGTGCTGATTGCCACTCATTTC
GTCGACCTGATTGAGGATTTTTGGCAGACCACACAGGGCATGAGCCAGATCGCTGACGCACT
GAGAGCCGTGATTCCACCCACTACCACACCCGTCCCTGATGGATACTTAATTAGCCATAACG
AGGCCCAAGAATTACCTTTAAATGACTTATTTGTCCTGCAGGAGGAAAGAATCGTGAACTTC
CAACCAGACTATCCTATTACTGCCAGAATTCACACCCACTTAAGAGTCTACACCAAGTTAAA
CGAGCAAGCCCTGGACAAGGCCAGGCGCCTGCTGTGGTGGCATTACAACTGCCTGCTGTGGG
GCGAGAGTAACGTGACTAATTATATCTCACGACTGCGGACATGGCTGAGCACTCCAGAAAAA
TACCGGGGGAAGGATGCTCCTACTATCGAGGCAATTACCCGCCCAATTCAGGTCGCCCAGGG
CTCTCGGAACCAGACCAAAGGAGTGCGGAAGCCTAGAGGCCTGGAGCCACGACGGAGAAAAG
TGAAGACTACCGTGGTCTATGGCAGGCGCCGATCTAAGAGTAGAGGGCGGAGAAGCTCCCCC
AGTCAGAGGGCAGGGTCACCAATCCCAAGAAACAGAGAAAATCAGAGCAGGTCCTCCAGCCC
ACGAGAATAA
SEQ ID NO: 114
S1.3 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspLysGluArgGln
AspHisArgArgArgLysAlaLeuGluAsnLysArgLysGlnLeuAlaAlaGlyGlyLys
HisLeuSerLysGluGluGluGluGluLeuLysArgLeuThrGluGluAspGluArgArg
GluArgArgThrAlaGlyProSerValGlyGlyValAsnProLeuGluGlyGlySerArg
GlyAlaProGlyGlyGlyPheValProAsnMetLeuSerValProGluSerProPheSer
ArgThrGlyGluGlyLeuAspValArgGlyAsnGlnGlyPheProMetAspValAsnAla
SerArgAlaLeuAlaAsnValTyrAspLeuProAspAspPhePheProGlnIleAspAsp
LeuValArgAspAlaLysAspAlaLeuGluProTyrTrpLysAlaGluThrIleLysLys
HisValLeuIleAlaThrHisPheValAspLeuIleGluAspPheTrpGlnThrThrGln
GlyMetSerGlnIleAlaAspAlaLeuArgAlaValIleProProThrThrThrProVal
ProAspGlyTyrLeuIleSerHisAsnGluAlaGlnGluLeuProLeuAsnAspLeuPhe
ValLeuGlnGluGluArgIleValAsnPheGlnProAspTyrProIleThrAlaArgIle
HisThrHisLeuArgValTyrThrLysLeuAsnGluGlnAlaLeuAspLysAlaArgArg
LeuLeuTrpTrpHisTyrAsnCysLeuLeuTrpGlyGluSerAsnValThrAsnTyrIle
SerArgLeuArgThrTrpLeuSerThrProGluLysTyrArgGlyLysAspAlaProThr
IleGluAlaIleThrArgProIleGlnValAlaGlnGlySerArgAsnGlnThrLysGly
ValArgLysProArgGlyLeuGluProArgArgArgLysValLysThrThrValValTyr
GlyArgArgArgSerLysSerArgGlyArgArgSerSerProSerGlnArgAlaGlySer
ProIleProArgAsnArgGluAsnGlnSerArgSerSerSerProArgGlu
SEQ ID NO: 115
S-HDAg-fusion-SHBcAg (S2.3 - gt2)
ATGGGACAGCCTGATAGTAGGAGACCACGGAGAGGGAGAGAGGAGTCACTGGGAAAATGGAT
TGATGCTCGAAGACGGAAGGAGGAACTGGAGCGAGACCTGCGGAAAGTGAATAAGACTATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACTGACCAGATGGAGGTGGATAG
CGGCCCCCGGAAAAGAAAGCACCCTGGCGGGTTCACCGAACAGGAGCGGAGAGACCATAGGC
GCCGAAAGGCCCTGGAGAATAAGAAAAAGCAGCTGAGCTCCGGAGGCAAAGACCTGTCCCGC
GAGGAAGAGGAAGAGCTGCGGAGACTGACCGAAGAGGATGAAAGGCGAGAGCGACGAGTCGC
AGGACCAAGAGTGGGCGACGTGAACCCCCTGGATGGAGGACCAAGGGGAGCACCTGGAGGAG
GATTCGTGCCCAGTATGCAGGGAATCCCAGAATCACCCTTTACAAGAAGGGGAGACGGCCTG
GATACACGGGGCACTCAGGAGTTCCCTATGGATGTCAATGCCAGTCGCGCCCTGGCTAACGT
GTACGACCTGCCAGACGATTTCTTTCCCCAGATCGACGATCTGGTGCGGGACGCTAAGGATG
CACTGGAACCATATTGGAAAGCCGAGACCATCAAAAAGCACGTGCTGATTGCTACACATTTC
GTCGACCTGATTGAGGATTTTTGGCAGACCACACAGGGAATGAGTCAGATCGCAGACGCCCT
GAGAGCTGTGATTCCACCCACTACCACACCCGTCCCTGATGGATACTTAATTTCCCATAACG
AGGCCCAAGAATTACCTTTAAATGACTTATTTGTCCTGCAGGAGGAAAGAATCGTGAACTTC
CAACCAGACTATCCTATTACCGCCAGAATTCACACTCACTTAAGAGTCTACACTAAACTCAA
CGAGCAAGCCCTGGACAAGGCCCGCCGACTGCTGTGGTGGCATTACAACTGCCTGCTGTGGG
GCGAGAGCAACGTGACAAATTATATCTCCAGGCTGCGCACCTGGCTGTCTACACCTGAAAAG
TACAGGGGCAAAGATGCACCTACAATCGAGGCCATTACTAGACCAATTCAGGTCGCCCAGGG
GTCTAGGAACCAGACAAAGGGGGTGAGAAAACCTAGGGGACTGGAGCCACGGAGAAGGAAGG
TGAAGACTACCGTGGTCTATGGACGCCGACGGTCAAAGAGCCGCGGCAGAAGGTCTAGTCCC
AGCCAGCGGGCAGGCAGCCCAATCCCACGCAACAGAGAAAATCAGTCACGGTCCAGCAGCCC
CAGAGAATAA
SEQ ID NO: 116
S2.3 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlnGluArgArg
AspHisArgArgArgLysAlaLeuGluAsnLysLysLysGlnLeuSerSerGlyGlyLys
AspLeuSerArgGluGluGluGluGluLeuArgArgLeuThrGluGluAspGluArgArg
GluArgArgValAlaGlyProArgValGlyAspValAsnProLeuAspGlyGlyProArg
GlyAlaProGlyGlyGlyPheValProSerMetGlnGlyIleProGluSerProPheThr
ArgArgGlyAspGlyLeuAspThrArgGlyThrGlnGluPheProMetAspValAsnAla
SerArgAlaLeuAlaAsnValTyrAspLeuProAspAspPhePheProGlnIleAspAsp
LeuValArgAspAlaLysAspAlaLeuGluProTyrTrpLysAlaGluThrIleLysLys
HisValLeuIleAlaThrHisPheValAspLeuIleGluAspPheTrpGlnThrThrGln
GlyMetSerGlnIleAlaAspAlaLeuArgAlaValIleProProThrThrThrProVal
ProAspGlyTyrLeuIleSerHisAsnGluAlaGlnGluLeuProLeuAsnAspLeuPhe
ValLeuGlnGluGluArgIleValAsnPheGlnProAspTyrProIleThrAlaArgIle
HisThrHisLeuArgValTyrThrLysLeuAsnGluGlnAlaLeuAspLysAlaArgArg
LeuLeuTrpTrpHisTyrAsnCysLeuLeuTrpGlyGluSerAsnValThrAsnTyrIle
SerArgLeuArgThrTrpLeuSerThrProGluLysTyrArgGlyLysAspAlaProThr
IleGluAlaIleThrArgProIleGlnValAlaGlnGlySerArgAsnGlnThrLysGly
ValArgLysProArgGlyLeuGluProArgArgArgLysValLysThrThrValValTyr
GlyArgArgArgSerLysSerArgGlyArgArgSerSerProSerGlnArgAlaGlySer
ProIleProArgAsnArgGluAsnGlnSerArgSerSerSerProArgGlu
SEQ ID NO: 117
frag-L-HDAg (L1.4 - gt1)
ATGGGGAGGAGCGAGTCAAAAAGGAACAGGGATGGGAGGGAAGGCATTCTGGAACAGTGGGT
CAACGGACGGAAAAAACTGGAGGATCTGGAAAGAGAGGCTAGGAAGATCAAGAAGAAAATCA
AGAAGTTAGAGGATGAAAATCCTTGGTTAGGAAACATCAAAGGAATCCTCGGAAAACGCGAC
AAGGATGGAGAAGGCGCCCCCCCTGCTAAAAGGGCACGCACAGACCAGATGGAGATCGATAG
CGGACCAGGAAAGCGACCACTGCGAGGAGGGTTTTCCGACGGAAGCGGAGCTACTAACTTCA
GCCTGCTGAAGCAGGCTGGAGACGTGGAGGAGAACCCTGGACCTAAAGAACGCCAGGATCAC
CGGAGAAGGAAGGCCCTGGAGAACAAGCGGAAGCAGCTGGCCGCTGGAGGCAAACATCTGTC
CAAGGAGGAAGAGGAAGAGCTGAAGCGCCTGACCGAAGAGGACGAACGCCGAGAGCGGAGAA
CAGCTGGACCAAGTGTGGGAGGAGTCAATCCCCTGGAGGGAGGATCACGGGGAGCACCTGGA
GGAGGATTTGTGCCAAACATGCTGTCTGTCCCTGAATCACCATTCAGCCGAACAGGCGAGGG
GCTGGATGTGAGGGGCAATCAGGGGTTCCCCTGGGACATCCTGTTTCCAGCAGATCCACCCT
TCTCCCCTCAGTCTTGCAGGCCACAGTAA
SEQ ID NO: 118
L1.4 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspGlySerGlyAla
ThrAsnPheSerLeuLeuLysGlnAlaGlyAspValGluGluAsnProGlyProLysGlu
ArgGlnAspHisArgArgArgLysAlaLeuGluAsnLysArgLysGlnLeuAlaAlaGly
GlyLysHisLeuSerLysGluGluGluGluGluLeuLysArgLeuThrGluGluAspGlu
ArgArgGluArgArgThrAlaGlyProSerValGlyGlyValAsnProLeuGluGlyGly
SerArgGlyAlaProGlyGlyGlyPheValProAsnMetLeuSerValProGluSerPro
PheSerArgThrGlyGluGlyLeuAspValArgGlyAsnGlnGlyPheProTrpAspIle
LeuPheProAlaAspProProPheSerProGlnSerCysArgProGln
SEQ ID NO: 119
frag-L-HDAg (L2.4 - gt2)
ATGGGACAGCCCGATAGCAGAAGACCTAGAAGAGGGAGGGAAGAAAGCCTGGGGAAATGGAT
TGACGCCCGAAGAAGGAAAGAAGAACTGGAGCGAGACCTGCGGAAAGTGAATAAGACAATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACAGACCAGATGGAGGTGGATAG
CGGCCCCCGGAAAAGAAAGCACCCTGGCGGGTTCACTGAAGGAAGCGGAGCTACTAACTTCA
GCCTGCTGAAGCAGGCTGGAGACGTGGAGGAGAACCCTGGACCTCAGGAGCGGAGAGACCAT
AGGCGCCGAAAGGCCCTGGAGAATAAGAAAAAGCAGCTGAGCTCCGGAGGCAAAGACCTGTC
CAGGGAGGAAGAGGAAGAGCTGCGGAGACTGACTGAAGAGGATGAAAGGCGAGAGCGACGAG
TCGCAGGACCTAGAGTGGGCGACGTGAACCCACTGGATGGAGGACCTAGGGGAGCACCAGGA
GGAGGATTCGTGCCTAGTATGCAGGGAATCCCCGAATCACCTTTTACCAGAAGGGGAGACGG
CCTGGATACACGCGGCACTCAGGAGTTCCCCTGGGTGAATCCTCAGCCACCACCTCCACGAC
TGCCACTGCTGGAATGCACCCCCCAGTAA
SEQ ID NO: 120
L2.4 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlySerGlyAla
ThrAsnPheSerLeuLeuLysGlnAlaGlyAspValGluGluAsnProGlyProGlnGlu
ArgArgAspHisArgArgArgLysAlaLeuGluAsnLysLysLysGlnLeuSerSerGly
GlyLysAspLeuSerArgGluGluGluGluGluLeuArgArgLeuThrGluGluAspGlu
ArgArgGluArgArgValAlaGlyProArgValGlyAspValAsnProLeuAspGlyGly
ProArgGlyAlaProGlyGlyGlyPheValProSerMetGlnGlyIleProGluSerPro
PheThrArgArgGlyAspGlyLeuAspThrArgGlyThrGlnGluPheProTrpValAsn
ProGlnProProProProArgLeuProLeuLeuGluCysThrProGln
SEQ ID NO: 121
frag-S-HDAg (S1.4 - gt1)
ATGGGGCGGAGCGAGTCAAAGAGAAATAGGGACGGGAGAGAAGGCATCCTGGAGCAGTGGGT
CAATGGAAGAAAGAAACTGGAAGATCTGGAAAGAGAGGCAAGGAAGATCAAGAAGAAAATCA
AAAAATTAGAGGATGAAAATCCATGGTTAGGAAACATCAAAGGAATCCTCGGAAAAAGAGAC
AAGGATGGAGAAGGCGCCCCCCCTGCTAAAAGGGCACGCACAGACCAGATGGAGATCGATAG
TGGACCAGGAAAGCGACCTCTGCGAGGAGGGTTCTCAGACGGAAGCGGAGCTACTAACTTCA
GCCTGCTGAAGCAGGCTGGAGACGTGGAGGAGAACCCTGGACCTAAAGAACGCCAGGATCAC
CGGAGAAGGAAGGCCCTGGAGAACAAGCGGAAGCAGCTGGCCGCTGGAGGCAAACATCTGAG
CAAGGAGGAAGAGGAAGAGCTGAAGCGCCTGACCGAAGAGGATGAACGCCGAGAGCGGAGAA
CAGCTGGACCATCCGTGGGAGGAGTCAATCCCCTGGAGGGAGGATCTCGGGGAGCACCAGGA
GGAGGGTTCGTGCCTAACATGCTGAGCGTCCCAGAATCACCCTTTAGCCGAACTGGAGAGGG
GCTGGACGTGAGGGGCAATCAGGGGTTCCCCTAA
SEQ ID NO: 122
S1.4 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspGlySerGlyAla
ThrAsnPheSerLeuLeuLysGlnAlaGlyAspValGluGluAsnProGlyProLysGlu
ArgGlnAspHisArgArgArgLysAlaLeuGluAsnLysArgLysGlnLeuAlaAlaGly
GlyLysHisLeuSerLysGluGluGluGluGluLeuLysArgLeuThrGluGluAspGlu
ArgArgGluArgArgThrAlaGlyProSerValGlyGlyValAsnProLeuGluGlyGly
SerArgGlyAlaProGlyGlyGlyPheValProAsnMetLeuSerValProGluSerPro
PheSerArgThrGlyGluGlyLeuAspValArgGlyAsnGlnGlyPhePro
SEQ ID NO: 123
frag-S-HDAg (S2.4 - gt2)
ATGGGACAGCCTGATAGTAGGAGACCACGGAGAGGGAGAGAGGAGTCACTGGGAAAATGGAT
TGATGCTCGAAGACGGAAGGAGGAACTGGAGCGAGACCTGCGGAAAGTGAATAAGACTATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACTGACCAGATGGAGGTGGATAG
CGGCCCCCGGAAAAGAAAGCACCCTGGCGGGTTCACCGAAGGAAGCGGAGCTACTAACTTCA
GCCTGCTGAAGCAGGCTGGAGACGTGGAGGAGAACCCTGGACCTCAGGAGCGGAGAGACCAT
AGGCGCCGAAAGGCCCTGGAGAATAAGAAAAAGCAGCTGAGCTCCGGAGGCAAAGACCTGTC
CCGCGAGGAAGAGGAAGAGCTGCGGAGACTGACCGAAGAGGATGAAAGGCGAGAGCGACGAG
TCGCAGGACCAAGAGTGGGCGACGTGAACCCCCTGGATGGAGGACCAAGGGGAGCACCTGGA
GGAGGATTCGTGCCCAGTATGCAGGGAATCCCAGAATCACCCTTTACAAGAAGGGGAGACGG
CCTGGATACACGGGGCACTCAGGAGTTCCCTTAA
SEQ ID NO: 124
S2.4 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlySerGlyAla
ThrAsnPheSerLeuLeuLysGlnAlaGlyAspValGluGluAsnProGlyProGlnGlu
ArgArgAspHisArgArgArgLysAlaLeuGluAsnLysLysLysGlnLeuSerSerGly
GlyLysAspLeuSerArgGluGluGluGluGluLeuArgArgLeuThrGluGluAspGlu
ArgArgGluArgArgValAlaGlyProArgValGlyAspValAsnProLeuAspGlyGly
ProArgGlyAlaProGlyGlyGlyPheValProSerMetGlnGlyIleProGluSerPro
PheThrArgArgGlyAspGlyLeuAspThrArgGlyThrGlnGluPhePro
SEQ ID NO: 125
frag-L-HDAg-fusion-SHBcAg (L1.5 - gt1)
ATGGGGAGGAGCGAGTCAAAAAGGAACAGGGATGGGAGGGAAGGCATTCTGGAACAGTGGGT
CAACGGACGGAAAAAACTGGAGGATCTGGAAAGAGAGGCTAGGAAGATCAAGAAGAAAATCA
AGAAGTTAGAGGATGAAAATCCTTGGTTAGGAAACATCAAAGGAATCCTCGGAAAACGCGAC
AAGGATGGAGAAGGCGCCCCCCCTGCTAAAAGGGCACGCACAGACCAGATGGAGATCGATAG
CGGACCAGGAAAGCGACCACTGCGAGGAGGGTTTTCCGACGGAAGCGGAGCTACTAACTTCA
GCCTGCTGAAGCAGGCTGGAGACGTGGAGGAGAACCCTGGACCTAAAGAACGCCAGGATCAC
CGGAGAAGGAAGGCCCTGGAGAACAAGCGGAAGCAGCTGGCCGCTGGAGGCAAACATCTGTC
CAAGGAGGAAGAGGAAGAGCTGAAGCGCCTGACCGAAGAGGACGAACGCCGAGAGCGGAGAA
CAGCTGGACCAAGTGTGGGAGGAGTCAATCCCCTGGAGGGAGGATCACGGGGAGCACCTGGA
GGAGGATTTGTGCCAAACATGCTGTCTGTCCCTGAATCACCATTCAGCCGAACAGGCGAGGG
GCTGGATGTGAGGGGCAATCAGGGGTTCCCCTGGGACATCCTGTTTCCAGCAGATCCACCCT
TCTCCCCTCAGTCTTGCAGGCCACAGATGGATGTCAATGCCAGCAGAGCACTGGCCAACGTG
TACGACCTGCCCGACGATTTCTTTCCTCAGATCGACGATCTGGTGAGGGACGCCAAAGATGC
TCTGGAACCCTATTGGAAGGCAGAGACAATCAAAAAGCACGTGCTGATTGCCACTCATTTTG
TCGACCTGATTGAGGATTTCTGGCAGACCACACAGGGCATGTCTCAGATCGCTGACGCACTG
AGAGCCGTGATTCCTCCAACTACCACACCAGTCCCCGATGGATATCTGATTTCCCATAACGA
GGCCCAAGAATTACCACTCAATGACTTATTCGTCCTGCAGGAGGAAAGAATCGTGAACTTTC
AACCTGACTATCCAATTACTGCTAGAATTCACACCCACTTAAGAGTCTACACCAAGTTAAAC
GAGCAAGCCCTGGACAAGGCCAGGCGCCTGCTGTGGTGGCATTACAACTGTCTGCTGTGGGG
CGAGAGCAACGTGACCAATTATATCTCCCGACTGCGGACATGGCTGTCTACTCCAGAAAAAT
ACAGAGGGAAGGATGCTCCTACTATCGAGGCAATTACCCGACCAATTCAGGTCGCACAGGGA
AGCCGGAACCAGACCAAAGGAGTGCGGAAGCCAAGAGGCCTGGAGCCACGACGGAGAAAAGT
GAAGACTACCGTGGTCTATGGCAGGCGCCGATCTAAGAGTAGAGGGCGGAGAAGCTCCCCTA
GCCAGAGGGCAGGAAGCCCCATCCCCAGAAACAGAGAAAACCAGTCCAGAAGCAGCAGCCCA
AGAGAGTAA
SEQ ID NO: 126
L1.5 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspGlySerGlyAla
ThrAsnPheSerLeuLeuLysGlnAlaGlyAspValGluGluAsnProGlyProLysGlu
ArgGlnAspHisArgArgArgLysAlaLeuGluAsnLysArgLysGlnLeuAlaAlaGly
GlyLysHisLeuSerLysGluGluGluGluGluLeuLysArgLeuThrGluGluAspGlu
ArgArgGluArgArgThrAlaGlyProSerValGlyGlyValAsnProLeuGluGlyGly
SerArgGlyAlaProGlyGlyGlyPheValProAsnMetLeuSerValProGluSerPro
PheSerArgThrGlyGluGlyLeuAspValArgGlyAsnGlnGlyPheProTrpAspIle
LeuPheProAlaAspProProPheSerProGlnSerCysArgProGlnMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyMetSerGlnIleAlaAspAlaLeuArgAlaValIleProProThrThrThrPro
ValProAspGlyTyrLeuIleSerHisAsnGluAlaGlnGluLeuProLeuAsnAspLeu
PheValLeuGlnGluGluArgIleValAsnPheGlnProAspTyrProIleThrAlaArg
IleHisThrHisLeuArgValTyrThrLysLeuAsnGluGlnAlaLeuAspLysAlaArg
ArgLeuLeuTrpTrpHisTyrAsnCysLeuLeuTrpGlyGluSerAsnValThrAsnTyr
IleSerArgLeuArgThrTrpLeuSerThrProGluLysTyrArgGlyLysAspAlaPro
ThrIleGluAlaIleThrArgProIleGlnValAlaGlnGlySerArgAsnGlnThrLys
GlyValArgLysProArgGlyLeuGluProArgArgArgLysValLysThrThrValVal
TyrGlyArgArgArgSerLysSerArgGlyArgArgSerSerProSerGlnArgAlaGly
SerProIleProArgAsnArgGluAsnGlnSerArgSerSerSerProArgGlu
SEQ ID NO: 127
frag-L-HDAg-fusion-SHBcAg (L2.5 - gt2)
ATGGGACAGCCCGATAGCAGAAGACCTAGAAGAGGGAGGGAAGAAAGCCTGGGGAAATGGAT
TGACGCCCGAAGAAGGAAAGAAGAACTGGAGCGAGACCTGCGGAAAGTGAATAAGACAATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACAGACCAGATGGAGGTGGATAG
CGGCCCCCGGAAAAGAAAGCACCCTGGCGGGTTCACTGAAGGAAGCGGAGCTACTAACTTCA
GCCTGCTGAAGCAGGCTGGAGACGTGGAGGAGAACCCTGGACCTCAGGAGCGGAGAGACCAT
AGGCGCCGAAAGGCCCTGGAGAATAAGAAAAAGCAGCTGAGCTCCGGAGGCAAAGACCTGTC
CAGGGAGGAAGAGGAAGAGCTGCGGAGACTGACTGAAGAGGATGAAAGGCGAGAGCGACGAG
TCGCAGGACCTAGAGTGGGCGACGTGAACCCACTGGATGGAGGACCTAGGGGAGCACCAGGA
GGAGGATTCGTGCCTAGTATGCAGGGAATCCCCGAATCACCTTTTACCAGAAGGGGAGACGG
CCTGGATACACGCGGCACTCAGGAGTTCCCCTGGGTGAATCCTCAGCCACCACCTCCACGAC
TGCCACTGCTGGAATGCACCCCCCAGATGGATGTCAATGCCAGTAGAGCCCTGGCTAACGTG
TACGACCTGCCAGACGATTTCTTTCCCCAGATCGACGATCTGGTGAGGGACGCTAAGGATGC
ACTGGAACCATATTGGAAAGCCGAGACTATCAAAAAGCACGTGCTGATTGCTACCCATTTCG
TCGACCTGATTGAGGATTTTTGGCAGACCACACAGGGAATGAGTCAGATCGCAGACGCACTG
CGAGCTGTGATTCCACCTACTACCACACCTGTCCCAGATGGATACTTAATTTCCCATAACGA
GGCCCAAGAATTACCTTTAAATGACTTATTTGTCCTGCAGGAGGAAAGAATCGTGAACTTCC
AACCTGACTATCCTATTACCGCCAGAATTCACACTCACTTAAGAGTGTACACTAAACTCAAC
GAACAAGCCCTGGACAAGGCCCGCCGACTGCTGTGGTGGCATTACAACTGTCTGCTGTGGGG
CGAAAGCAACGTGACAAATTATATCTCCAGGCTGCGCACCTGGCTGTCTACACCTGAAAAGT
ACCGGGGCAAAGATGCACCCACAATCGAGGCCATTACTAGACCTATTCAGGTCGCCCAGGGG
TCTAGGAACCAGACCAAGGGGGTGAGAAAACCTAGGGGACTGGAGCCACGGAGAAGGAAGGT
GAAGACTACCGTGGTCTATGGACGCCGACGGTCAAAGAGCCGCGGCAGAAGGTCTAGTCCAA
GCCAGCGGGCAGGGTCACCAATCCCCAGAAACCGAGAGAATCAGTCCAGGTCATCATCACCA
AGAGAATAA
SEQ ID NO: 128
L2.5 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlySerGlyAla
ThrAsnPheSerLeuLeuLysGlnAlaGlyAspValGluGluAsnProGlyProGlnGlu
ArgArgAspHisArgArgArgLysAlaLeuGluAsnLysLysLysGlnLeuSerSerGly
GlyLysAspLeuSerArgGluGluGluGluGluLeuArgArgLeuThrGluGluAspGlu
ArgArgGluArgArgValAlaGlyProArgValGlyAspValAsnProLeuAspGlyGly
ProArgGlyAlaProGlyGlyGlyPheValProSerMetGlnGlyIleProGluSerPro
PheThrArgArgGlyAspGlyLeuAspThrArgGlyThrGlnGluPheProTrpValAsn
ProGlnProProProProArgLeuProLeuLeuGluCysThrProGlnMetAspValAsn
AlaSerArgAlaLeuAlaAsnValTyrAspLeuProAspAspPhePheProGlnIleAsp
AspLeuValArgAspAlaLysAspAlaLeuGluProTyrTrpLysAlaGluThrIleLys
LysHisValLeuIleAlaThrHisPheValAspLeuIleGluAspPheTrpGlnThrThr
GlnGlyMetSerGlnIleAlaAspAlaLeuArgAlaValIleProProThrThrThrPro
ValProAspGlyTyrLeuIleSerHisAsnGluAlaGlnGluLeuProLeuAsnAspLeu
PheValLeuGlnGluGluArgIleValAsnPheGlnProAspTyrProIleThrAlaArg
IleHisThrHisLeuArgValTyrThrLysLeuAsnGluGlnAlaLeuAspLysAlaArg
ArgLeuLeuTrpTrpHisTyrAsnCysLeuLeuTrpGlyGluSerAsnValThrAsnTyr
IleSerArgLeuArgThrTrpLeuSerThrProGluLysTyrArgGlyLysAspAlaPro
ThrIleGluAlaIleThrArgProIleGlnValAlaGlnGlySerArgAsnGlnThrLys
GlyValArgLysProArgGlyLeuGluProArgArgArgLysValLysThrThrValVal
TyrGlyArgArgArgSerLysSerArgGlyArgArgSerSerProSerGlnArgAlaGly
SerProIleProArgAsnArgGluAsnGlnSerArgSerSerSerProArgGlu
SEQ ID NO: 129
frag-S-HDAg-fusion-SHBcAg (S1.5 - gt1)
ATGGGGCGGAGCGAGTCAAAGAGAAATAGGGACGGGAGAGAAGGCATCCTGGAGCAGTGGGT
CAATGGAAGAAAGAAACTGGAAGATCTGGAAAGAGAGGCAAGGAAGATCAAGAAGAAAATCA
AAAAATTAGAGGATGAAAATCCATGGTTAGGAAACATCAAAGGAATCCTCGGAAAAAGAGAC
AAGGATGGAGAAGGCGCCCCCCCTGCTAAAAGGGCACGCACAGACCAGATGGAGATCGATAG
TGGACCAGGAAAGCGACCTCTGCGAGGAGGGTTCTCAGACGGAAGCGGAGCTACTAACTTCA
GCCTGCTGAAGCAGGCTGGAGACGTGGAGGAGAACCCTGGACCTAAAGAACGCCAGGATCAC
CGGAGAAGGAAGGCCCTGGAGAACAAGCGGAAGCAGCTGGCCGCTGGAGGCAAACATCTGAG
CAAGGAGGAAGAGGAAGAGCTGAAGCGCCTGACCGAAGAGGATGAACGCCGAGAGCGGAGAA
CAGCTGGACCATCCGTGGGAGGAGTCAATCCCCTGGAGGGAGGATCTCGGGGAGCACCAGGA
GGAGGGTTCGTGCCTAACATGCTGAGCGTCCCAGAATCACCCTTTAGCCGAACTGGAGAGGG
GCTGGACGTGAGGGGCAATCAGGGGTTCCCCATGGATGTCAATGCCTCCAGAGCACTGGCCA
ACGTGTACGATCTGCCAGACGATTTCTTTCCCCAGATCGACGATCTGGTGAGGGACGCCAAA
GATGCTCTGGAACCTTATTGGAAGGCAGAGACAATCAAAAAGCACGTGCTGATTGCCACTCA
TTTCGTCGACCTGATTGAGGATTTTTGGCAGACCACACAGGGCATGAGCCAGATCGCTGACG
CACTGAGAGCCGTGATTCCACCCACTACCACACCCGTCCCTGATGGATACTTAATTAGCCAT
AACGAGGCCCAAGAATTACCTTTAAATGACTTATTTGTCCTGCAGGAGGAAAGAATCGTGAA
CTTCCAACCAGACTATCCTATTACTGCCAGAATTCACACCCACTTAAGAGTCTACACCAAGT
TAAACGAGCAAGCCCTGGACAAGGCCAGGCGCCTGCTGTGGTGGCATTACAACTGCCTGCTG
TGGGGCGAGAGTAACGTGACTAATTATATCTCACGACTGCGGACATGGCTGAGCACTCCAGA
AAAATACCGGGGGAAGGATGCTCCTACTATCGAGGCAATTACCCGCCCAATTCAGGTCGCCC
AGGGCTCTCGGAACCAGACCAAAGGAGTGCGGAAGCCTAGAGGCCTGGAGCCACGACGGAGA
AAAGTGAAGACTACCGTGGTCTATGGCAGGCGCCGATCTAAGAGTAGAGGGCGGAGAAGCTC
CCCCAGTCAGAGGGCAGGGTCACCAATCCCAAGAAACAGAGAAAATCAGAGCAGGTCCTCCA
GCCCACGAGAATAA
SEQ ID NO: 130
S1.5 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspGlySerGlyAla
ThrAsnPheSerLeuLeuLysGlnAlaGlyAspValGluGluAsnProGlyProLysGlu
ArgGlnAspHisArgArgArgLysAlaLeuGluAsnLysArgLysGlnLeuAlaAlaGly
GlyLysHisLeuSerLysGluGluGluGluGluLeuLysArgLeuThrGluGluAspGlu
ArgArgGluArgArgThrAlaGlyProSerValGlyGlyValAsnProLeuGluGlyGly
SerArgGlyAlaProGlyGlyGlyPheValProAsnMetLeuSerValProGluSerPro
PheSerArgThrGlyGluGlyLeuAspValArgGlyAsnGlnGlyPheProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyMetSerGlnIleAlaAspAlaLeuArgAlaValIleProProThrThrThr
ProValProAspGlyTyrLeuIleSerHisAsnGluAlaGlnGluLeuProLeuAsnAsp
LeuPheValLeuGlnGluGluArgIleValAsnPheGlnProAspTyrProIleThrAla
ArgIleHisThrHisLeuArgValTyrThrLysLeuAsnGluGlnAlaLeuAspLysAla
ArgArgLeuLeuTrpTrpHisTyrAsnCysLeuLeuTrpGlyGluSerAsnValThrAsn
TyrIleSerArgLeuArgThrTrpLeuSerThrProGluLysTyrArgGlyLysAspAla
ProThrIleGluAlaIleThrArgProIleGlnValAlaGlnGlySerArgAsnGlnThr
LysGlyValArgLysProArgGlyLeuGluProArgArgArgLysValLysThrThrVal
ValTyrGlyArgArgArgSerLysSerArgGlyArgArgSerSerProSerGlnArgAla
GlySerProIleProArgAsnArgGluAsnGlnSerArgSerSerSerProArgGlu
SEQ ID NO: 131
frag-S-HDAg-fusion-SHBcAg (S2.5 - gt2)
ATGGGACAGCCTGATAGTAGGAGACCACGGAGAGGGAGAGAGGAGTCACTGGGAAAATGGAT
TGATGCTCGAAGACGGAAGGAGGAACTGGAGCGAGACCTGCGGAAAGTGAATAAGACTATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACTGACCAGATGGAGGTGGATAG
CGGCCCCCGGAAAAGAAAGCACCCTGGCGGGTTCACCGAAGGAAGCGGAGCTACTAACTTCA
GCCTGCTGAAGCAGGCTGGAGACGTGGAGGAGAACCCTGGACCTCAGGAGCGGAGAGACCAT
AGGCGCCGAAAGGCCCTGGAGAATAAGAAAAAGCAGCTGAGCTCCGGAGGCAAAGACCTGTC
CCGCGAGGAAGAGGAAGAGCTGCGGAGACTGACCGAAGAGGATGAAAGGCGAGAGCGACGAG
TCGCAGGACCAAGAGTGGGCGACGTGAACCCCCTGGATGGAGGACCAAGGGGAGCACCTGGA
GGAGGATTCGTGCCCAGTATGCAGGGAATCCCAGAATCACCCTTTACAAGAAGGGGAGACGG
CCTGGATACACGGGGCACTCAGGAGTTCCCTATGGATGTCAATGCCAGTCGCGCCCTGGCTA
ACGTGTACGACCTGCCAGACGATTTCTTTCCCCAGATCGACGATCTGGTGCGGGACGCTAAG
GATGCACTGGAACCATATTGGAAAGCCGAGACCATCAAAAAGCACGTGCTGATTGCTACACA
TTTCGTCGACCTGATTGAGGATTTTTGGCAGACCACACAGGGAATGAGTCAGATCGCAGACG
CCCTGAGAGCTGTGATTCCACCCACTACCACACCCGTCCCTGATGGATACTTAATTTCCCAT
AACGAGGCCCAAGAATTACCTTTAAATGACTTATTTGTCCTGCAGGAGGAAAGAATCGTGAA
CTTCCAACCAGACTATCCTATTACCGCCAGAATTCACACTCACTTAAGAGTCTACACTAAAC
TCAACGAGCAAGCCCTGGACAAGGCCCGCCGACTGCTGTGGTGGCATTACAACTGCCTGCTG
TGGGGCGAGAGCAACGTGACAAATTATATCTCCAGGCTGCGCACCTGGCTGTCTACACCTGA
AAAGTACAGGGGCAAAGATGCACCTACAATCGAGGCCATTACTAGACCAATTCAGGTCGCCC
AGGGGTCTAGGAACCAGACAAAGGGGGTGAGAAAACCTAGGGGACTGGAGCCACGGAGAAGG
AAGGTGAAGACTACCGTGGTCTATGGACGCCGACGGTCAAAGAGCCGCGGCAGAAGGTCTAG
TCCCAGCCAGCGGGCAGGCAGCCCAATCCCACGCAACAGAGAAAATCAGTCACGGTCCAGCA
GCCCCAGAGAATAA
SEQ ID NO: 132
S2.5 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlySerGlyAla
ThrAsnPheSerLeuLeuLysGlnAlaGlyAspValGluGluAsnProGlyProGlnGlu
ArgArgAspHisArgArgArgLysAlaLeuGluAsnLysLysLysGlnLeuSerSerGly
GlyLysAspLeuSerArgGluGluGluGluGluLeuArgArgLeuThrGluGluAspGlu
ArgArgGluArgArgValAlaGlyProArgValGlyAspValAsnProLeuAspGlyGly
ProArgGlyAlaProGlyGlyGlyPheValProSerMetGlnGlyIleProGluSerPro
PheThrArgArgGlyAspGlyLeuAspThrArgGlyThrGlnGluPheProMetAspVal
AsnAlaSerArgAlaLeuAlaAsnValTyrAspLeuProAspAspPhePheProGlnIle
AspAspLeuValArgAspAlaLysAspAlaLeuGluProTyrTrpLysAlaGluThrIle
LysLysHisValLeuIleAlaThrHisPheValAspLeuIleGluAspPheTrpGlnThr
ThrGlnGlyMetSerGlnIleAlaAspAlaLeuArgAlaValIleProProThrThrThr
ProValProAspGlyTyrLeuIleSerHisAsnGluAlaGlnGluLeuProLeuAsnAsp
LeuPheValLeuGlnGluGluArgIleValAsnPheGlnProAspTyrProIleThrAla
ArgIleHisThrHisLeuArgValTyrThrLysLeuAsnGluGlnAlaLeuAspLysAla
ArgArgLeuLeuTrpTrpHisTyrAsnCysLeuLeuTrpGlyGluSerAsnValThrAsn
TyrIleSerArgLeuArgThrTrpLeuSerThrProGluLysTyrArgGlyLysAspAla
ProThrIleGluAlaIleThrArgProIleGlnValAlaGlnGlySerArgAsnGlnThr
LysGlyValArgLysProArgGlyLeuGluProArgArgArgLysValLysThrThrVal
ValTyrGlyArgArgArgSerLysSerArgGlyArgArgSerSerProSerGlnArgAla
GlySerProIleProArgAsnArgGluAsnGlnSerArgSerSerSerProArgGlu
SEQ ID NO: 133
frag-L-HDAg-SHBcAg (L1.6 - gt1)
ATGGGGAGGAGCGAGTCAAAAAGGAACAGGGATGGGAGGGAAGGCATTCTGGAACAGTGGGT
CAACGGACGGAAAAAACTGGAGGATCTGGAAAGAGAGGCTAGGAAGATCAAGAAGAAAATCA
AGAAGTTAGAGGATGAAAATCCTTGGTTAGGAAACATCAAAGGAATCCTCGGAAAACGCGAC
AAGGATGGAGAAGGCGCCCCCCCTGCTAAAAGGGCACGCACAGACCAGATGGAGATCGATAG
CGGACCAGGAAAGCGACCACTGCGAGGAGGGTTTTCCGACGGAAGCGGAGCTACTAACTTCA
GCCTGCTGAAGCAGGCTGGAGACGTGGAGGAGAACCCTGGACCTAAAGAACGCCAGGATCAC
CGGAGAAGGAAGGCCCTGGAGAACAAGCGGAAGCAGCTGGCCGCTGGAGGCAAACATCTGTC
CAAGGAGGAAGAGGAAGAGCTGAAGCGCCTGACCGAAGAGGACGAACGCCGAGAGCGGAGAA
CAGCTGGACCAAGTGTGGGAGGAGTCAATCCCCTGGAGGGAGGATCACGGGGAGCACCTGGA
GGAGGATTTGTGCCAAACATGCTGTCTGTCCCTGAATCACCATTCAGCCGAACAGGCGAGGG
GCTGGATGTGAGGGGCAATCAGGGGTTCCCCTGGGACATCCTGTTTCCAGCAGATCCACCCT
TCTCCCCTCAGTCTTGCAGGCCACAGGGAAGCGGAGCTACTAACTTCAGCCTGCTGAAGCAG
GCTGGAGACGTGGAGGAGAACCCTGGACCTATGGATGTCAATGCCAGCAGAGCACTGGCCAA
CGTGTACGACCTGCCCGACGATTTCTTTCCTCAGATCGACGATCTGGTGAGGGACGCCAAAG
ATGCTCTGGAACCCTATTGGAAGGCAGAGACAATCAAAAAGCACGTGCTGATTGCCACTCAT
TTTGTCGACCTGATTGAGGATTTCTGGCAGACCACACAGGGCATGTCTCAGATCGCTGACGC
ACTGAGAGCCGTGATTCCTCCAACTACCACACCAGTCCCCGATGGATATCTGATTTCCCATA
ACGAGGCCCAAGAATTACCACTCAATGACTTATTCGTCCTGCAGGAGGAAAGAATCGTGAAC
TTTCAACCTGACTATCCAATTACTGCTAGAATTCACACCCACTTAAGAGTCTACACCAAGTT
AAACGAGCAAGCCCTGGACAAGGCCAGGCGCCTGCTGTGGTGGCATTACAACTGTCTGCTGT
GGGGCGAGAGCAACGTGACCAATTATATCTCCCGACTGCGGACATGGCTGTCTACTCCAGAA
AAATACAGAGGGAAGGATGCTCCTACTATCGAGGCAATTACCCGACCAATTCAGGTCGCACA
GGGAAGCCGGAACCAGACCAAAGGAGTGCGGAAGCCAAGAGGCCTGGAGCCACGACGGAGAA
AAGTGAAGACTACCGTGGTCTATGGCAGGCGCCGATCTAAGAGTAGAGGGCGGAGAAGCTCC
CCTAGCCAGAGGGCAGGAAGCCCCATCCCCAGAAACAGAGAAAACCAGTCCAGAAGCAGCAG
CCCAAGAGAGTAA
SEQ ID NO: 134
L1.6 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspGlySerGlyAla
ThrAsnPheSerLeuLeuLysGlnAlaGlyAspValGluGluAsnProGlyProLysGlu
ArgGlnAspHisArgArgArgLysAlaLeuGluAsnLysArgLysGlnLeuAlaAlaGly
GlyLysHisLeuSerLysGluGluGluGluGluLeuLysArgLeuThrGluGluAspGlu
ArgArgGluArgArgThrAlaGlyProSerValGlyGlyValAsnProLeuGluGlyGly
SerArgGlyAlaProGlyGlyGlyPheValProAsnMetLeuSerValProGluSerPro
PheSerArgThrGlyGluGlyLeuAspValArgGlyAsnGlnGlyPheProTrpAspIle
LeuPheProAlaAspProProPheSerProGlnSerCysArgProGlnGlySerGlyAla
ThrAsnPheSerLeuLeuLysGlnAlaGlyAspValGluGluAsnProGlyProMetAsp
ValAsnAlaSerArgAlaLeuAlaAsnValTyrAspLeuProAspAspPhePheProGln
IleAspAspLeuValArgAspAlaLysAspAlaLeuGluProTyrTrpLysAlaGluThr
IleLysLysHisValLeuIleAlaThrHisPheValAspLeuIleGluAspPheTrpGln
ThrThrGlnGlyMetSerGlnIleAlaAspAlaLeuArgAlaValIleProProThrThr
ThrProValProAspGlyTyrLeuIleSerHisAsnGluAlaGlnGluLeuProLeuAsn
AspLeuPheValLeuGlnGluGluArgIleValAsnPheGlnProAspTyrProIleThr
AlaArgIleHisThrHisLeuArgValTyrThrLysLeuAsnGluGlnAlaLeuAspLys
AlaArgArgLeuLeuTrpTrpHisTyrAsnCysLeuLeuTrpGlyGluSerAsnValThr
AsnTyrIleSerArgLeuArgThrTrpLeuSerThrProGluLysTyrArgGlyLysAsp
AlaProThrIleGluAlaIleThrArgProIleGlnValAlaGlnGlySerArgAsnGln
ThrLysGlyValArgLysProArgGlyLeuGluProArgArgArgLysValLysThrThr
ValValTyrGlyArgArgArgSerLysSerArgGlyArgArgSerSerProSerGlnArg
AlaGlySerProIleProArgAsnArgGluAsnGlnSerArgSerSerSerProArgGlu
SEQ ID NO: 135
frag-L-HDAg-SHBcAg (L2.6 - gt2)
ATGGGACAGCCCGATAGCAGAAGACCTAGAAGAGGGAGGGAAGAAAGCCTGGGGAAATGGAT
TGACGCCCGAAGAAGGAAAGAAGAACTGGAGCGAGACCTGCGGAAAGTGAATAAGACAATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACAGACCAGATGGAGGTGGATAG
CGGCCCCCGGAAAAGAAAGCACCCTGGCGGGTTCACTGAAGGAAGCGGAGCTACTAACTTCA
GCCTGCTGAAGCAGGCTGGAGACGTGGAGGAGAACCCTGGACCTCAGGAGCGGAGAGACCAT
AGGCGCCGAAAGGCCCTGGAGAATAAGAAAAAGCAGCTGAGCTCCGGAGGCAAAGACCTGTC
CAGGGAGGAAGAGGAAGAGCTGCGGAGACTGACTGAAGAGGATGAAAGGCGAGAGCGACGAG
TCGCAGGACCTAGAGTGGGCGACGTGAACCCACTGGATGGAGGACCTAGGGGAGCACCAGGA
GGAGGATTCGTGCCTAGTATGCAGGGAATCCCCGAATCACCTTTTACCAGAAGGGGAGACGG
CCTGGATACACGCGGCACTCAGGAGTTCCCCTGGGTGAATCCTCAGCCACCACCTCCACGAC
TGCCACTGCTGGAATGCACCCCCCAGGGAAGCGGAGCTACTAACTTCAGCCTGCTGAAGCAG
GCTGGAGACGTGGAGGAGAACCCTGGACCTATGGATGTCAATGCCAGTAGAGCCCTGGCTAA
CGTGTACGACCTGCCAGACGATTTCTTTCCCCAGATCGACGATCTGGTGAGGGACGCTAAGG
ATGCACTGGAACCATATTGGAAAGCCGAGACTATCAAAAAGCACGTGCTGATTGCTACCCAT
TTCGTCGACCTGATTGAGGATTTTTGGCAGACCACACAGGGAATGAGTCAGATCGCAGACGC
ACTGCGAGCTGTGATTCCACCTACTACCACACCTGTCCCAGATGGATACTTAATTTCCCATA
ACGAGGCCCAAGAATTACCTTTAAATGACTTATTTGTCCTGCAGGAGGAAAGAATCGTGAAC
TTCCAACCTGACTATCCTATTACCGCCAGAATTCACACTCACTTAAGAGTGTACACTAAACT
CAACGAACAAGCCCTGGACAAGGCCCGCCGACTGCTGTGGTGGCATTACAACTGTCTGCTGT
GGGGCGAAAGCAACGTGACAAATTATATCTCCAGGCTGCGCACCTGGCTGTCTACACCTGAA
AAGTACCGGGGCAAAGATGCACCCACAATCGAGGCCATTACTAGACCTATTCAGGTCGCCCA
GGGGTCTAGGAACCAGACCAAGGGGGTGAGAAAACCTAGGGGACTGGAGCCACGGAGAAGGA
AGGTGAAGACTACCGTGGTCTATGGACGCCGACGGTCAAAGAGCCGCGGCAGAAGGTCTAGT
CCAAGCCAGCGGGCAGGGTCACCAATCCCCAGAAACCGAGAGAATCAGTCCAGGTCATCATC
ACCAAGAGAATAA
SEQ ID NO: 136
L2.6 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlySerGlyAla
ThrAsnPheSerLeuLeuLysGlnAlaGlyAspValGluGluAsnProGlyProGlnGlu
ArgArgAspHisArgArgArgLysAlaLeuGluAsnLysLysLysGlnLeuSerSerGly
GlyLysAspLeuSerArgGluGluGluGluGluLeuArgArgLeuThrGluGluAspGlu
ArgArgGluArgArgValAlaGlyProArgValGlyAspValAsnProLeuAspGlyGly
ProArgGlyAlaProGlyGlyGlyPheValProSerMetGlnGlyIleProGluSerPro
PheThrArgArgGlyAspGlyLeuAspThrArgGlyThrGlnGluPheProTrpValAsn
ProGlnProProProProArgLeuProLeuLeuGluCysThrProGlnGlySerGlyAla
ThrAsnPheSerLeuLeuLysGlnAlaGlyAspValGluGluAsnProGlyProMetAsp
ValAsnAlaSerArgAlaLeuAlaAsnValTyrAspLeuProAspAspPhePheProGln
IleAspAspLeuValArgAspAlaLysAspAlaLeuGluProTyrTrpLysAlaGluThr
IleLysLysHisValLeuIleAlaThrHisPheValAspLeuIleGluAspPheTrpGln
ThrThrGlnGlyMetSerGlnIleAlaAspAlaLeuArgAlaValIleProProThrThr
ThrProValProAspGlyTyrLeuIleSerHisAsnGluAlaGlnGluLeuProLeuAsn
AspLeuPheValLeuGlnGluGluArgIleValAsnPheGlnProAspTyrProIleThr
AlaArgIleHisThrHisLeuArgValTyrThrLysLeuAsnGluGlnAlaLeuAspLys
AlaArgArgLeuLeuTrpTrpHisTyrAsnCysLeuLeuTrpGlyGluSerAsnValThr
AsnTyrIleSerArgLeuArgThrTrpLeuSerThrProGluLysTyrArgGlyLysAsp
AlaProThrIleGluAlaIleThrArgProIleGlnValAlaGlnGlySerArgAsnGln
ThrLysGlyValArgLysProArgGlyLeuGluProArgArgArgLysValLysThrThr
ValValTyrGlyArgArgArgSerLysSerArgGlyArgArgSerSerProSerGlnArg
AlaGlySerProIleProArgAsnArgGluAsnGlnSerArgSerSerSerProArgGlu
SEQ ID NO: 137
frag-S-HDAg-SHBcAg (S1.6 - gt1)
ATGGGGCGGAGCGAGTCAAAGAGAAATAGGGACGGGAGAGAAGGCATCCTGGAGCAGTGGGT
CAATGGAAGAAAGAAACTGGAAGATCTGGAAAGAGAGGCAAGGAAGATCAAGAAGAAAATCA
AAAAATTAGAGGATGAAAATCCATGGTTAGGAAACATCAAAGGAATCCTCGGAAAAAGAGAC
AAGGATGGAGAAGGCGCCCCCCCTGCTAAAAGGGCACGCACAGACCAGATGGAGATCGATAG
TGGACCAGGAAAGCGACCTCTGCGAGGAGGGTTCTCAGACGGAAGCGGAGCTACTAACTTCA
GCCTGCTGAAGCAGGCTGGAGACGTGGAGGAGAACCCTGGACCTAAAGAACGCCAGGATCAC
CGGAGAAGGAAGGCCCTGGAGAACAAGCGGAAGCAGCTGGCCGCTGGAGGCAAACATCTGAG
CAAGGAGGAAGAGGAAGAGCTGAAGCGCCTGACCGAAGAGGATGAACGCCGAGAGCGGAGAA
CAGCTGGACCATCCGTGGGAGGAGTCAATCCCCTGGAGGGAGGATCTCGGGGAGCACCAGGA
GGAGGGTTCGTGCCTAACATGCTGAGCGTCCCAGAATCACCCTTTAGCCGAACTGGAGAGGG
GCTGGACGTGAGGGGCAATCAGGGGTTCCCCGGAAGCGGAGCTACTAACTTCAGCCTGCTGA
AGCAGGCTGGAGACGTGGAGGAGAACCCTGGACCTATGGATGTCAATGCCTCCAGAGCACTG
GCCAACGTGTACGATCTGCCAGACGATTTCTTTCCCCAGATCGACGATCTGGTGAGGGACGC
CAAAGATGCTCTGGAACCTTATTGGAAGGCAGAGACAATCAAAAAGCACGTGCTGATTGCCA
CTCATTTCGTCGACCTGATTGAGGATTTTTGGCAGACCACACAGGGCATGAGCCAGATCGCT
GACGCACTGAGAGCCGTGATTCCACCCACTACCACACCCGTCCCTGATGGATACTTAATTAG
CCATAACGAGGCCCAAGAATTACCTTTAAATGACTTATTTGTCCTGCAGGAGGAAAGAATCG
TGAACTTCCAACCAGACTATCCTATTACTGCCAGAATTCACACCCACTTAAGAGTCTACACC
AAGTTAAACGAGCAAGCCCTGGACAAGGCCAGGCGCCTGCTGTGGTGGCATTACAACTGCCT
GCTGTGGGGCGAGAGTAACGTGACTAATTATATCTCACGACTGCGGACATGGCTGAGCACTC
CAGAAAAATACCGGGGGAAGGATGCTCCTACTATCGAGGCAATTACCCGCCCAATTCAGGTC
GCCCAGGGCTCTCGGAACCAGACCAAAGGAGTGCGGAAGCCTAGAGGCCTGGAGCCACGACG
GAGAAAAGTGAAGACTACCGTGGTCTATGGCAGGCGCCGATCTAAGAGTAGAGGGCGGAGAA
GCTCCCCCAGTCAGAGGGCAGGGTCACCAATCCCAAGAAACAGAGAAAATCAGAGCAGGTCC
TCCAGCCCACGAGAATAA
SEQ ID NO: 138
S1.6 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspGlySerGlyAla
ThrAsnPheSerLeuLeuLysGlnAlaGlyAspValGluGluAsnProGlyProLysGlu
ArgGlnAspHisArgArgArgLysAlaLeuGluAsnLysArgLysGlnLeuAlaAlaGly
GlyLysHisLeuSerLysGluGluGluGluGluLeuLysArgLeuThrGluGluAspGlu
ArgArgGluArgArgThrAlaGlyProSerValGlyGlyValAsnProLeuGluGlyGly
SerArgGlyAlaProGlyGlyGlyPheValProAsnMetLeuSerValProGluSerPro
PheSerArgThrGlyGluGlyLeuAspValArgGlyAsnGlnGlyPheProGlySerGly
AlaThrAsnPheSerLeuLeuLysGlnAlaGlyAspValGluGluAsnProGlyProMet
AspValAsnAlaSerArgAlaLeuAlaAsnValTyrAspLeuProAspAspPhePhePro
GlnIleAspAspLeuValArgAspAlaLysAspAlaLeuGluProTyrTrpLysAlaGlu
ThrIleLysLysHisValLeuIleAlaThrHisPheValAspLeuIleGluAspPheTrp
GlnThrThrGlnGlyMetSerGlnIleAlaAspAlaLeuArgAlaValIleProProThr
ThrThrProValProAspGlyTyrLeuIleSerHisAsnGluAlaGlnGluLeuProLeu
AsnAspLeuPheValLeuGlnGluGluArgIleValAsnPheGlnProAspTyrProIle
ThrAlaArgIleHisThrHisLeuArgValTyrThrLysLeuAsnGluGlnAlaLeuAsp
LysAlaArgArgLeuLeuTrpTrpHisTyrAsnCysLeuLeuTrpGlyGluSerAsnVal
ThrAsnTyrIleSerArgLeuArgThrTrpLeuSerThrProGluLysTyrArgGlyLys
AspAlaProThrIleGluAlaIleThrArgProIleGlnValAlaGlnGlySerArgAsn
GlnThrLysGlyValArgLysProArgGlyLeuGluProArgArgArgLysValLysThr
ThrValValTyrGlyArgArgArgSerLysSerArgGlyArgArgSerSerProSerGln
ArgAlaGlySerProIleProArgAsnArgGluAsnGlnSerArgSerSerSerProArg
Glu
SEQ ID NO: 139
frag-S-HDAg-SHBcAg (S2.6 - gt2)
ATGGGACAGCCTGATAGTAGGAGACCACGGAGAGGGAGAGAGGAGTCACTGGGAAAATGGAT
TGATGCTCGAAGACGGAAGGAGGAACTGGAGCGAGACCTGCGGAAAGTGAATAAGACTATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACTGACCAGATGGAGGTGGATAG
CGGCCCCCGGAAAAGAAAGCACCCTGGCGGGTTCACCGAAGGAAGCGGAGCTACTAACTTCA
GCCTGCTGAAGCAGGCTGGAGACGTGGAGGAGAACCCTGGACCTCAGGAGCGGAGAGACCAT
AGGCGCCGAAAGGCCCTGGAGAATAAGAAAAAGCAGCTGAGCTCCGGAGGCAAAGACCTGTC
CCGCGAGGAAGAGGAAGAGCTGCGGAGACTGACCGAAGAGGATGAAAGGCGAGAGCGACGAG
TCGCAGGACCAAGAGTGGGCGACGTGAACCCCCTGGATGGAGGACCAAGGGGAGCACCTGGA
GGAGGATTCGTGCCCAGTATGCAGGGAATCCCAGAATCACCCTTTACAAGAAGGGGAGACGG
CCTGGATACACGGGGCACTCAGGAGTTCCCTGGAAGCGGAGCTACTAACTTCAGCCTGCTGA
AGCAGGCTGGAGACGTGGAGGAGAACCCTGGACCTATGGATGTCAATGCCAGTCGCGCCCTG
GCTAACGTGTACGACCTGCCAGACGATTTCTTTCCCCAGATCGACGATCTGGTGCGGGACGC
TAAGGATGCACTGGAACCATATTGGAAAGCCGAGACCATCAAAAAGCACGTGCTGATTGCTA
CACATTTCGTCGACCTGATTGAGGATTTTTGGCAGACCACACAGGGAATGAGTCAGATCGCA
GACGCCCTGAGAGCTGTGATTCCACCCACTACCACACCCGTCCCTGATGGATACTTAATTTC
CCATAACGAGGCCCAAGAATTACCTTTAAATGACTTATTTGTCCTGCAGGAGGAAAGAATCG
TGAACTTCCAACCAGACTATCCTATTACCGCCAGAATTCACACTCACTTAAGAGTCTACACT
AAACTCAACGAGCAAGCCCTGGACAAGGCCCGCCGACTGCTGTGGTGGCATTACAACTGCCT
GCTGTGGGGCGAGAGCAACGTGACAAATTATATCTCCAGGCTGCGCACCTGGCTGTCTACAC
CTGAAAAGTACAGGGGCAAAGATGCACCTACAATCGAGGCCATTACTAGACCAATTCAGGTC
GCCCAGGGGTCTAGGAACCAGACAAAGGGGGTGAGAAAACCTAGGGGACTGGAGCCACGGAG
AAGGAAGGTGAAGACTACCGTGGTCTATGGACGCCGACGGTCAAAGAGCCGCGGCAGAAGGT
CTAGTCCCAGCCAGCGGGCAGGCAGCCCAATCCCACGCAACAGAGAAAATCAGTCACGGTCC
AGCAGCCCCAGAGAATAA
SEQ ID NO: 140
S2.6 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlySerGlyAla
ThrAsnPheSerLeuLeuLysGlnAlaGlyAspValGluGluAsnProGlyProGlnGlu
ArgArgAspHisArgArgArgLysAlaLeuGluAsnLysLysLysGlnLeuSerSerGly
GlyLysAspLeuSerArgGluGluGluGluGluLeuArgArgLeuThrGluGluAspGlu
ArgArgGluArgArgValAlaGlyProArgValGlyAspValAsnProLeuAspGlyGly
ProArgGlyAlaProGlyGlyGlyPheValProSerMetGlnGlyIleProGluSerPro
PheThrArgArgGlyAspGlyLeuAspThrArgGlyThrGlnGluPheProGlySerGly
AlaThrAsnPheSerLeuLeuLysGlnAlaGlyAspValGluGluAsnProGlyProMet
AspValAsnAlaSerArgAlaLeuAlaAsnValTyrAspLeuProAspAspPhePhePro
GlnIleAspAspLeuValArgAspAlaLysAspAlaLeuGluProTyrTrpLysAlaGlu
ThrIleLysLysHisValLeuIleAlaThrHisPheValAspLeuIleGluAspPheTrp
GlnThrThrGlnGlyMetSerGlnIleAlaAspAlaLeuArgAlaValIleProProThr
ThrThrProValProAspGlyTyrLeuIleSerHisAsnGluAlaGlnGluLeuProLeu
AsnAspLeuPheValLeuGlnGluGluArgIleValAsnPheGlnProAspTyrProIle
ThrAlaArgIleHisThrHisLeuArgValTyrThrLysLeuAsnGluGlnAlaLeuAsp
LysAlaArgArgLeuLeuTrpTrpHisTyrAsnCysLeuLeuTrpGlyGluSerAsnVal
ThrAsnTyrIleSerArgLeuArgThrTrpLeuSerThrProGluLysTyrArgGlyLys
AspAlaProThrIleGluAlaIleThrArgProIleGlnValAlaGlnGlySerArgAsn
GlnThrLysGlyValArgLysProArgGlyLeuGluProArgArgArgLysValLysThr
ThrValValTyrGlyArgArgArgSerLysSerArgGlyArgArgSerSerProSerGln
ArgAlaGlySerProIleProArgAsnArgGluAsnGlnSerArgSerSerSerProArg
Glu
SEQ ID NO: 141
frag-L-HDAg-frag-SHBcAg (L1.7 - gt1)
ATGGGGAGGAGCGAGTCAAAAAGGAACAGGGATGGGAGGGAAGGCATTCTGGAACAGTGGGT
CAACGGACGGAAAAAACTGGAGGATCTGGAAAGAGAGGCTAGGAAGATCAAGAAGAAAATCA
AGAAGTTAGAGGATGAAAATCCTTGGTTAGGAAACATCAAAGGAATCCTCGGAAAACGCGAC
AAGGATGGAGAAGGCGCCCCCCCTGCTAAAAGGGCACGCACAGACCAGATGGAGATCGATAG
CGGACCAGGAAAGCGACCACTGCGAGGAGGGTTTTCCGACGGAAGCGGAGCTACTAACTTCA
GCCTGCTGAAGCAGGCTGGAGACGTGGAGGAGAACCCTGGACCTAAAGAACGCCAGGATCAC
CGGAGAAGGAAGGCCCTGGAGAACAAGCGGAAGCAGCTGGCCGCTGGAGGCAAACATCTGTC
CAAGGAGGAAGAGGAAGAGCTGAAGCGCCTGACCGAAGAGGACGAACGCCGAGAGCGGAGAA
CAGCTGGACCAAGTGTGGGAGGAGTCAATCCCCTGGAGGGAGGATCACGGGGAGCACCTGGA
GGAGGATTTGTGCCAAACATGCTGTCTGTCCCTGAATCACCATTCAGCCGAACAGGCGAGGG
GCTGGATGTGAGGGGCAATCAGGGGTTCCCCTGGGACATCCTGTTTCCAGCAGATCCACCCT
TCTCCCCTCAGTCTTGCAGGCCACAGGGAAGCGGAGCTACTAACTTCAGCCTGCTGAAGCAG
GCTGGAGACGTGGAGGAGAACCCTGGACCTATGGATGTCAATGCCAGCAGAGCACTGGCCAA
CGTGTACGACCTGCCCGACGATTTCTTTCCTCAGATCGACGATCTGGTGAGGGACGCCAAAG
ATGCTCTGGAACCCTATTGGAAGGCAGAGACAATCAAAAAGCACGTGCTGATTGCCACTCAT
TTTGTCGACCTGATTGAGGATTTCTGGCAGACCACACAGGGAGGAAGCGGAGCTACTAACTT
CAGCCTGCTGAAGCAGGCTGGAGACGTGGAGGAGAACCCTGGACCTATGAGTCAGATCGCTG
ATGCACTGAGAGCTGTGATTCCTCCAACTACCACACCTGTCCCAGATGGATACTTAATTTCC
CATAACGAGGCTCAAGAATTACCACTAAATGACTTATTCGTCCTGCAGGAGGAAAGAATCGT
GAACTTTCAACCTGACTATCCAATTACTGCTAGAATTCACACACATCTGCGCGTCTATGGAA
GCGGAGCTACTAACTTCAGCCTGCTGAAGCAGGCTGGAGACGTGGAGGAGAACCCTGGACCT
ACCAAGTTAAATGAACAAGCCCTGGACAAGGCCAGGCGCCTGCTGTGGTGGCATTACAATTG
TCTGCTGTGGGGCGAGTCCAACGTGACCAATTATATCTCTCGACTGCGGACATGGCTGAGTA
CTCCAGAAAAATACAGAGGGAAGGACGCTCCTACTATCGAGGCAATTACCAGGCCAATTCAG
GTGGCCCAGGGCGGAAGCGGAGCTACTAACTTCAGCCTGCTGAAGCAGGCTGGAGACGTGGA
GGAGAACCCTGGACCTTCCAGAAATCAGACTAAAGGAGTCCGGAAGCCAAGAGGCCTGGAGC
CACGACGGAGAAAAGTGAAGACTACCGTGGTCTATGGCAGGCGCCGATCTAAGAGTCGCGGG
CGGAGAAGCTCCCCTTCTCAGCGGGCAGGGTCACCTATCCCACGGAACAGAGAGAACCAGAG
CAGAAGCAGCAGTCCAAGAGAGTAA
SEQ ID NO: 142
L1.7 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspGlySerGlyAla
ThrAsnPheSerLeuLeuLysGlnAlaGlyAspValGluGluAsnProGlyProLysGlu
ArgGlnAspHisArgArgArgLysAlaLeuGluAsnLysArgLysGlnLeuAlaAlaGly
GlyLysHisLeuSerLysGluGluGluGluGluLeuLysArgLeuThrGluGluAspGlu
ArgArgGluArgArgThrAlaGlyProSerValGlyGlyValAsnProLeuGluGlyGly
SerArgGlyAlaProGlyGlyGlyPheValProAsnMetLeuSerValProGluSerPro
PheSerArgThrGlyGluGlyLeuAspValArgGlyAsnGlnGlyPheProTrpAspIle
LeuPheProAlaAspProProPheSerProGlnSerCysArgProGlnGlySerGlyAla
ThrAsnPheSerLeuLeuLysGlnAlaGlyAspValGluGluAsnProGlyProMetAsp
ValAsnAlaSerArgAlaLeuAlaAsnValTyrAspLeuProAspAspPhePheProGln
IleAspAspLeuValArgAspAlaLysAspAlaLeuGluProTyrTrpLysAlaGluThr
IleLysLysHisValLeuIleAlaThrHisPheValAspLeuIleGluAspPheTrpGln
ThrThrGlnGlyGlySerGlyAlaThrAsnPheSerLeuLeuLysGlnAlaGlyAspVal
GluGluAsnProGlyProMetSerGlnIleAlaAspAlaLeuArgAlaValIleProPro
ThrThrThrProValProAspGlyTyrLeuIleSerHisAsnGluAlaGlnGluLeuPro
LeuAsnAspLeuPheValLeuGlnGluGluArgIleValAsnPheGlnProAspTyrPro
IleThrAlaArgIleHisThrHisLeuArgValTyrGlySerGlyAlaThrAsnPheSer
LeuLeuLysGlnAlaGlyAspValGluGluAsnProGlyProThrLysLeuAsnGluGln
AlaLeuAspLysAlaArgArgLeuLeuTrpTrpHisTyrAsnCysLeuLeuTrpGlyGlu
SerAsnValThrAsnTyrIleSerArgLeuArgThrTrpLeuSerThrProGluLysTyr
ArgGlyLysAspAlaProThrIleGluAlaIleThrArgProIleGlnValAlaGlnGly
GlySerGlyAlaThrAsnPheSerLeuLeuLysGlnAlaGlyAspValGluGluAsnPro
GlyProSerArgAsnGlnThrLysGlyValArgLysProArgGlyLeuGluProArgArg
ArgLysValLysThrThrValValTyrGlyArgArgArgSerLysSerArgGlyArgArg
SerSerProSerGlnArgAlaGlySerProIleProArgAsnArgGluAsnGlnSerArg
SerSerSerProArgGlu
SEQ ID NO: 143
frag-L-HDAg-frag-SHBcAg (L2.7 - gt2)
ATGGGACAGCCCGATAGCAGAAGACCTAGAAGAGGGAGGGAAGAAAGCCTGGGGAAATGGAT
TGACGCCCGAAGAAGGAAAGAAGAACTGGAGCGAGACCTGCGGAAAGTGAATAAGACAATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACAGACCAGATGGAGGTGGATAG
CGGCCCCCGGAAAAGAAAGCACCCTGGCGGGTTCACTGAAGGAAGCGGAGCTACTAACTTCA
GCCTGCTGAAGCAGGCTGGAGACGTGGAGGAGAACCCTGGACCTCAGGAGCGGAGAGACCAT
AGGCGCCGAAAGGCCCTGGAGAATAAGAAAAAGCAGCTGAGCTCCGGAGGCAAAGACCTGTC
CAGGGAGGAAGAGGAAGAGCTGCGGAGACTGACTGAAGAGGATGAAAGGCGAGAGCGACGAG
TCGCAGGACCTAGAGTGGGCGACGTGAACCCACTGGATGGAGGACCTAGGGGAGCACCAGGA
GGAGGATTCGTGCCTAGTATGCAGGGAATCCCCGAATCACCTTTTACCAGAAGGGGAGACGG
CCTGGATACACGCGGCACTCAGGAGTTCCCCTGGGTGAATCCTCAGCCACCACCTCCACGAC
TGCCACTGCTGGAATGCACCCCCCAGGGAAGCGGAGCTACTAACTTCAGCCTGCTGAAGCAG
GCTGGAGACGTGGAGGAGAACCCTGGACCTATGGATGTCAATGCCAGCAGAGCACTGGCCAA
CGTGTACGACCTGCCCGACGATTTCTTTCCTCAGATCGACGATCTGGTGAGGGACGCCAAAG
ATGCTCTGGAACCCTATTGGAAGGCAGAGACAATCAAAAAGCACGTGCTGATTGCCACTCAT
TTTGTCGACCTGATTGAGGATTTCTGGCAGACCACACAGGGAGGAAGCGGAGCTACTAACTT
CAGCCTGCTGAAGCAGGCTGGAGACGTGGAGGAGAACCCTGGACCTATGAGTCAGATCGCTG
ATGCACTGAGAGCTGTGATTCCTCCAACTACCACACCTGTCCCAGATGGATACTTAATTTCC
CATAACGAGGCTCAAGAATTACCACTAAATGACTTATTCGTCCTGCAGGAGGAAAGAATCGT
GAACTTTCAACCTGACTATCCAATTACTGCTAGAATTCACACACATCTGCGCGTCTATGGAA
GCGGAGCTACTAACTTCAGCCTGCTGAAGCAGGCTGGAGACGTGGAGGAGAACCCTGGACCT
ACCAAGTTAAATGAACAAGCCCTGGACAAGGCCAGGCGCCTGCTGTGGTGGCATTACAATTG
TCTGCTGTGGGGCGAGTCCAACGTGACCAATTATATCTCTCGACTGCGGACATGGCTGAGTA
CTCCAGAAAAATACAGAGGGAAGGACGCTCCTACTATCGAGGCAATTACCAGGCCAATTCAG
GTGGCCCAGGGCGGAAGCGGAGCTACTAACTTCAGCCTGCTGAAGCAGGCTGGAGACGTGGA
GGAGAACCCTGGACCTTCCAGAAATCAGACTAAAGGAGTCCGGAAGCCAAGAGGCCTGGAGC
CACGACGGAGAAAAGTGAAGACTACCGTGGTCTATGGCAGGCGCCGATCTAAGAGTCGCGGG
CGGAGAAGCTCCCCTTCTCAGCGGGCAGGGTCACCTATCCCACGGAACAGAGAGAACCAGAG
CAGAAGCAGCAGTCCAAGAGAGTAA
SEQ ID NO: 144
L2.7 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlySerGlyAla
ThrAsnPheSerLeuLeuLysGlnAlaGlyAspValGluGluAsnProGlyProGlnGlu
ArgArgAspHisArgArgArgLysAlaLeuGluAsnLysLysLysGlnLeuSerSerGly
GlyLysAspLeuSerArgGluGluGluGluGluLeuArgArgLeuThrGluGluAspGlu
ArgArgGluArgArgValAlaGlyProArgValGlyAspValAsnProLeuAspGlyGly
ProArgGlyAlaProGlyGlyGlyPheValProSerMetGlnGlyIleProGluSerPro
PheThrArgArgGlyAspGlyLeuAspThrArgGlyThrGlnGluPheProTrpValAsn
ProGlnProProProProArgLeuProLeuLeuGluCysThrProGlnGlySerGlyAla
ThrAsnPheSerLeuLeuLysGlnAlaGlyAspValGluGluAsnProGlyProMetAsp
ValAsnAlaSerArgAlaLeuAlaAsnValTyrAspLeuProAspAspPhePheProGln
IleAspAspLeuValArgAspAlaLysAspAlaLeuGluProTyrTrpLysAlaGluThr
IleLysLysHisValLeuIleAlaThrHisPheValAspLeuIleGluAspPheTrpGln
ThrThrGlnGlyGlySerGlyAlaThrAsnPheSerLeuLeuLysGlnAlaGlyAspVal
GluGluAsnProGlyProMetSerGlnIleAlaAspAlaLeuArgAlaValIleProPro
ThrThrThrProValProAspGlyTyrLeuIleSerHisAsnGluAlaGlnGluLeuPro
LeuAsnAspLeuPheValLeuGlnGluGluArgIleValAsnPheGlnProAspTyrPro
IleThrAlaArgIleHisThrHisLeuArgValTyrGlySerGlyAlaThrAsnPheSer
LeuLeuLysGlnAlaGlyAspValGluGluAsnProGlyProThrLysLeuAsnGluGln
AlaLeuAspLysAlaArgArgLeuLeuTrpTrpHisTyrAsnCysLeuLeuTrpGlyGlu
SerAsnValThrAsnTyrIleSerArgLeuArgThrTrpLeuSerThrProGluLysTyr
ArgGlyLysAspAlaProThrIleGluAlaIleThrArgProIleGlnValAlaGlnGly
GlySerGlyAlaThrAsnPheSerLeuLeuLysGlnAlaGlyAspValGluGluAsnPro
GlyProSerArgAsnGlnThrLysGlyValArgLysProArgGlyLeuGluProArgArg
ArgLysValLysThrThrValValTyrGlyArgArgArgSerLysSerArgGlyArgArg
SerSerProSerGlnArgAlaGlySerProIleProArgAsnArgGluAsnGlnSerArg
SerSerSerProArgGlu
SEQ ID NO: 145
frag-S-HDAg-frag-SHBcAg (S1.7 - gt1)
ATGGGGCGGAGCGAGTCAAAGAGAAATAGGGACGGGAGAGAAGGCATCCTGGAGCAGTGGGT
CAATGGAAGAAAGAAACTGGAAGATCTGGAAAGAGAGGCAAGGAAGATCAAGAAGAAAATCA
AAAAATTAGAGGATGAAAATCCATGGTTAGGAAACATCAAAGGAATCCTCGGAAAAAGAGAC
AAGGATGGAGAAGGCGCCCCCCCTGCTAAAAGGGCACGCACAGACCAGATGGAGATCGATAG
TGGACCAGGAAAGCGACCTCTGCGAGGAGGGTTCTCAGACGGAAGCGGAGCTACTAACTTCA
GCCTGCTGAAGCAGGCTGGAGACGTGGAGGAGAACCCTGGACCTAAAGAACGCCAGGATCAC
CGGAGAAGGAAGGCCCTGGAGAACAAGCGGAAGCAGCTGGCCGCTGGAGGCAAACATCTGAG
CAAGGAGGAAGAGGAAGAGCTGAAGCGCCTGACCGAAGAGGATGAACGCCGAGAGCGGAGAA
CAGCTGGACCATCCGTGGGAGGAGTCAATCCCCTGGAGGGAGGATCTCGGGGAGCACCAGGA
GGAGGGTTCGTGCCTAACATGCTGAGCGTCCCAGAATCACCCTTTAGCCGAACTGGAGAGGG
GCTGGACGTGAGGGGCAATCAGGGGTTCCCCGGAAGCGGAGCTACTAACTTCAGCCTGCTGA
AGCAGGCTGGAGACGTGGAGGAGAACCCTGGACCTATGGATGTCAATGCCAGCAGAGCACTG
GCCAACGTGTACGACCTGCCCGACGATTTCTTTCCTCAGATCGACGATCTGGTGAGGGACGC
CAAAGATGCTCTGGAACCCTATTGGAAGGCAGAGACAATCAAAAAGCACGTGCTGATTGCCA
CTCATTTTGTCGACCTGATTGAGGATTTCTGGCAGACCACACAGGGAGGAAGCGGAGCTACT
AACTTCAGCCTGCTGAAGCAGGCTGGAGACGTGGAGGAGAACCCTGGACCTATGAGTCAGAT
CGCTGATGCACTGAGAGCTGTGATTCCTCCAACTACCACACCTGTCCCAGATGGATACTTAA
TTTCCCATAACGAGGCTCAAGAATTACCACTAAATGACTTATTCGTCCTGCAGGAGGAAAGA
ATCGTGAACTTTCAACCTGACTATCCAATTACTGCTAGAATTCACACACATCTGCGCGTCTA
TGGAAGCGGAGCTACTAACTTCAGCCTGCTGAAGCAGGCTGGAGACGTGGAGGAGAACCCTG
GACCTACCAAGTTAAATGAACAAGCCCTGGACAAGGCCAGGCGCCTGCTGTGGTGGCATTAC
AATTGTCTGCTGTGGGGCGAGTCCAACGTGACCAATTATATCTCTCGACTGCGGACATGGCT
GAGTACTCCAGAAAAATACAGAGGGAAGGACGCTCCTACTATCGAGGCAATTACCAGGCCAA
TTCAGGTGGCCCAGGGCGGAAGCGGAGCTACTAACTTCAGCCTGCTGAAGCAGGCTGGAGAC
GTGGAGGAGAACCCTGGACCTTCCAGAAATCAGACTAAAGGAGTCCGGAAGCCAAGAGGCCT
GGAGCCACGACGGAGAAAAGTGAAGACTACCGTGGTCTATGGCAGGCGCCGATCTAAGAGTC
GCGGGCGGAGAAGCTCCCCTTCTCAGCGGGCAGGGTCACCTATCCCACGGAACAGAGAGAAC
CAGAGCAGAAGCAGCAGTCCAAGAGAGTAA
SEQ ID NO: 146
S1.7 encoded protein
MetGlyArgSerGluSerLysArgAsnArgAspGlyArgGluGlyIleLeuGluGlnTrp
ValAsnGlyArgLysLysLeuGluAspLeuGluArgGluAlaArgLysIleLysLysLys
IleLysLysLeuGluAspGluAsnProTrpLeuGlyAsnIleLysGlyIleLeuGlyLys
ArgAspLysAspGlyGluGlyAlaProProAlaLysArgAlaArgThrAspGlnMetGlu
IleAspSerGlyProGlyLysArgProLeuArgGlyGlyPheSerAspGlySerGlyAla
ThrAsnPheSerLeuLeuLysGlnAlaGlyAspValGluGluAsnProGlyProLysGlu
ArgGlnAspHisArgArgArgLysAlaLeuGluAsnLysArgLysGlnLeuAlaAlaGly
GlyLysHisLeuSerLysGluGluGluGluGluLeuLysArgLeuThrGluGluAspGlu
ArgArgGluArgArgThrAlaGlyProSerValGlyGlyValAsnProLeuGluGlyGly
SerArgGlyAlaProGlyGlyGlyPheValProAsnMetLeuSerValProGluSerPro
PheSerArgThrGlyGluGlyLeuAspValArgGlyAsnGlnGlyPheProGlySerGly
AlaThrAsnPheSerLeuLeuLysGlnAlaGlyAspValGluGluAsnProGlyProMet
AspValAsnAlaSerArgAlaLeuAlaAsnValTyrAspLeuProAspAspPhePhePro
GlnIleAspAspLeuValArgAspAlaLysAspAlaLeuGluProTyrTrpLysAlaGlu
ThrIleLysLysHisValLeuIleAlaThrHisPheValAspLeuIleGluAspPheTrp
GlnThrThrGlnGlyGlySerGlyAlaThrAsnPheSerLeuLeuLysGlnAlaGlyAsp
ValGluGluAsnProGlyProMetSerGlnIleAlaAspAlaLeuArgAlaValIlePro
ProThrThrThrProValProAspGlyTyrLeuIleSerHisAsnGluAlaGlnGluLeu
ProLeuAsnAspLeuPheValLeuGlnGluGluArgIleValAsnPheGlnProAspTyr
ProIleThrAlaArgIleHisThrHisLeuArgValTyrGlySerGlyAlaThrAsnPhe
SerLeuLeuLysGlnAlaGlyAspValGluGluAsnProGlyProThrLysLeuAsnGlu
GlnAlaLeuAspLysAlaArgArgLeuLeuTrpTrpHisTyrAsnCysLeuLeuTrpGly
GluSerAsnValThrAsnTyrIleSerArgLeuArgThrTrpLeuSerThrProGluLys
TyrArgGlyLysAspAlaProThrIleGluAlaIleThrArgProIleGlnValAlaGln
GlyGlySerGlyAlaThrAsnPheSerLeuLeuLysGlnAlaGlyAspValGluGluAsn
ProGlyProSerArgAsnGlnThrLysGlyValArgLysProArgGlyLeuGluProArg
ArgArgLysValLysThrThrValValTyrGlyArgArgArgSerLysSerArgGlyArg
ArgSerSerProSerGlnArgAlaGlySerProIleProArgAsnArgGluAsnGlnSer
ArgSerSerSerProArgGlu
SEQ ID NO: 147
frag-S-HDAg-frag-SHBcAg (S2.7 - gt2)
ATGGGACAGCCTGATAGTAGGAGACCACGGAGAGGGAGAGAGGAGTCACTGGGAAAATGGAT
TGATGCTCGAAGACGGAAGGAGGAACTGGAGCGAGACCTGCGGAAAGTGAATAAGACTATCA
AAAGACTGGAGGAAGATAACCCCTGGCTGGGGAATATTCGCGGCATCATTGGGCGAAAAGAC
AAGGATGGAGAAGGAGCTCCACCTGCAAAGCGAGCACGAACTGACCAGATGGAGGTGGATAG
CGGCCCCCGGAAAAGAAAGCACCCTGGCGGGTTCACCGAAGGAAGCGGAGCTACTAACTTCA
GCCTGCTGAAGCAGGCTGGAGACGTGGAGGAGAACCCTGGACCTCAGGAGCGGAGAGACCAT
AGGCGCCGAAAGGCCCTGGAGAATAAGAAAAAGCAGCTGAGCTCCGGAGGCAAAGACCTGTC
CCGCGAGGAAGAGGAAGAGCTGCGGAGACTGACCGAAGAGGATGAAAGGCGAGAGCGACGAG
TCGCAGGACCAAGAGTGGGCGACGTGAACCCCCTGGATGGAGGACCAAGGGGAGCACCTGGA
GGAGGATTCGTGCCCAGTATGCAGGGAATCCCAGAATCACCCTTTACAAGAAGGGGAGACGG
CCTGGATACACGGGGCACTCAGGAGTTCCCTGGAAGCGGAGCTACTAACTTCAGCCTGCTGA
AGCAGGCTGGAGACGTGGAGGAGAACCCTGGACCTATGGATGTCAATGCCAGCAGAGCACTG
GCCAACGTGTACGACCTGCCCGACGATTTCTTTCCTCAGATCGACGATCTGGTGAGGGACGC
CAAAGATGCTCTGGAACCCTATTGGAAGGCAGAGACAATCAAAAAGCACGTGCTGATTGCCA
CTCATTTTGTCGACCTGATTGAGGATTTCTGGCAGACCACACAGGGAGGAAGCGGAGCTACT
AACTTCAGCCTGCTGAAGCAGGCTGGAGACGTGGAGGAGAACCCTGGACCTATGAGTCAGAT
CGCTGATGCACTGAGAGCTGTGATTCCTCCAACTACCACACCTGTCCCAGATGGATACTTAA
TTTCCCATAACGAGGCTCAAGAATTACCACTAAATGACTTATTCGTCCTGCAGGAGGAAAGA
ATCGTGAACTTTCAACCTGACTATCCAATTACTGCTAGAATTCACACACATCTGCGCGTCTA
TGGAAGCGGAGCTACTAACTTCAGCCTGCTGAAGCAGGCTGGAGACGTGGAGGAGAACCCTG
GACCTACCAAGTTAAATGAACAAGCCCTGGACAAGGCCAGGCGCCTGCTGTGGTGGCATTAC
AATTGTCTGCTGTGGGGCGAGTCCAACGTGACCAATTATATCTCTCGACTGCGGACATGGCT
GAGTACTCCAGAAAAATACAGAGGGAAGGACGCTCCTACTATCGAGGCAATTACCAGGCCAA
TTCAGGTGGCCCAGGGCGGAAGCGGAGCTACTAACTTCAGCCTGCTGAAGCAGGCTGGAGAC
GTGGAGGAGAACCCTGGACCTTCCAGAAATCAGACTAAAGGAGTCCGGAAGCCAAGAGGCCT
GGAGCCACGACGGAGAAAAGTGAAGACTACCGTGGTCTATGGCAGGCGCCGATCTAAGAGTC
GCGGGCGGAGAAGCTCCCCTTCTCAGCGGGCAGGGTCACCTATCCCACGGAACAGAGAGAAC
CAGAGCAGAAGCAGCAGTCCAAGAGAGTAA
SEQ ID NO: 148
S2.7 encoded protein
MetGlyGlnProAspSerArgArgProArgArgGlyArgGluGluSerLeuGlyLysTrp
IleAspAlaArgArgArgLysGluGluLeuGluArgAspLeuArgLysValAsnLysThr
IleLysArgLeuGluGluAspAsnProTrpLeuGlyAsnIleArgGlyIleIleGlyArg
LysAspLysAspGlyGluGlyAlaProProAlaLysArgAlaArgThrAspGlnMetGlu
ValAspSerGlyProArgLysArgLysHisProGlyGlyPheThrGluGlySerGlyAla
ThrAsnPheSerLeuLeuLysGlnAlaGlyAspValGluGluAsnProGlyProGlnGlu
ArgArgAspHisArgArgArgLysAlaLeuGluAsnLysLysLysGlnLeuSerSerGly
GlyLysAspLeuSerArgGluGluGluGluGluLeuArgArgLeuThrGluGluAspGlu
ArgArgGluArgArgValAlaGlyProArgValGlyAspValAsnProLeuAspGlyGly
ProArgGlyAlaProGlyGlyGlyPheValProSerMetGlnGlyIleProGluSerPro
PheThrArgArgGlyAspGlyLeuAspThrArgGlyThrGlnGluPheProGlySerGly
AlaThrAsnPheSerLeuLeuLysGlnAlaGlyAspValGluGluAsnProGlyProMet
AspValAsnAlaSerArgAlaLeuAlaAsnValTyrAspLeuProAspAspPhePhePro
GlnIleAspAspLeuValArgAspAlaLysAspAlaLeuGluProTyrTrpLysAlaGlu
ThrIleLysLysHisValLeuIleAlaThrHisPheValAspLeuIleGluAspPheTrp
GlnThrThrGlnGlyGlySerGlyAlaThrAsnPheSerLeuLeuLysGlnAlaGlyAsp
ValGluGluAsnProGlyProMetSerGlnIleAlaAspAlaLeuArgAlaValIlePro
ProThrThrThrProValProAspGlyTyrLeuIleSerHisAsnGluAlaGlnGluLeu
ProLeuAsnAspLeuPheValLeuGlnGluGluArgIleValAsnPheGlnProAspTyr
ProIleThrAlaArgIleHisThrHisLeuArgValTyrGlySerGlyAlaThrAsnPhe
SerLeuLeuLysGlnAlaGlyAspValGluGluAsnProGlyProThrLysLeuAsnGlu
GlnAlaLeuAspLysAlaArgArgLeuLeuTrpTrpHisTyrAsnCysLeuLeuTrpGly
GluSerAsnValThrAsnTyrIleSerArgLeuArgThrTrpLeuSerThrProGluLys
TyrArgGlyLysAspAlaProThrIleGluAlaIleThrArgProIleGlnValAlaGln
GlyGlySerGlyAlaThrAsnPheSerLeuLeuLysGlnAlaGlyAspValGluGluAsn
ProGlyProSerArgAsnGlnThrLysGlyValArgLysProArgGlyLeuGluProArg
ArgArgLysValLysThrThrValValTyrGlyArgArgArgSerLysSerArgGlyArg
ArgSerSerProSerGlnArgAlaGlySerProIleProArgAsnArgGluAsnGlnSer
ArgSerSerSerProArgGlu
```

1. An isolated nucleic acid comprising a nucleic acid sequence that encodes a hepatitis D virus (HDV) polypeptide, HDAg-L or HDAg-S, wherein said nucleic acid is codon optimized for expression in humans.

2-37. (canceled)

38. The isolated nucleic acid of claim 1, wherein said HDV polypeptide comprises a full length HDV polypeptide, a 150 amino acid fragment of an HDV polypeptide, a 100 amino acid fragment of an HDV polypeptide, or a 50 amino acid fragment of an HDV polypeptide.

39. The isolated nucleic acid of claim 1, wherein said nucleic acid further comprises a nucleic acid sequence encoding a self-cleavage sequence, P2A, E2A, F2A, or T2A.

40. The isolated nucleic acid of claim 39, wherein said self-cleavage sequence exists at the N or C terminus of said HDV polypeptide or within the sequence of said HDV polypeptide.

41. The isolated nucleic acid of claim 1, wherein said nucleic acid further comprises a nucleic acid sequence encoding an HBcAg polypeptide.

42. The isolated nucleic acid of claim 41, wherein said nucleic acid sequence encoding said HBcAg polypeptide is codon-optimized for expression in humans.

43. The isolated nucleic acid of claim 42, wherein said HBcAg polypeptide is a stork or heron HBcAg.

44. The isolated nucleic acid of claim 43, wherein said nucleic acid sequence encoding said HBcAg polypeptide further comprises a nucleic acid sequence encoding a self-cleavage polypeptide sequence, P2A, E2A, F2A, or T2A.

45. The isolated nucleic acid of claim 44, wherein a self-cleavage polypeptide sequence exists within said HBcAg polypeptide or antigenic portion thereof, at the N terminus of HBcAg polypeptide or antigenic portion thereof and/or at the C terminus of HBcAg polypeptide or antigenic portion thereof.

46. The isolated nucleic acid of claim 1, wherein the nucleic acid comprises the HDV polypeptide encoding sequence of SEQ ID NOs: 33, 35, 37, or 39.

47. The isolated nucleic acid of claim 41, wherein the nucleic acid comprises the HBcAg polypeptide encoding sequence of SEQ ID NOs: 1, 3, 5, 7, or 9.

48. The isolated nucleic acid of claim 44, wherein the nucleic acid comprises the self-cleavage polypeptide sequence encoded by SEQ ID NOs: 11, 13, 15, 17, or 21.

49. The isolated nucleic acid of claim 1, wherein the nucleic acid comprises the sequence of SEQ ID NO: 58, 60, 62, 64, 66, 68, 70, or 72.

50. A method of eliciting an immune response comprising administering to a subject having HDV infection and/or HBV infection the nucleic acid of claim 1.

51. The method of claim 50, wherein said administering comprises injecting said nucleic acid into a patient using an IVIN needle with or without electroporation.

52. The method of claim 51, further comprising administering a second administration of a nucleic acid of claim 1.

53. The method of claim 52, wherein said second administration is given one week, two weeks, three weeks, four weeks, five weeks, or six weeks after the first administration of said nucleic acid of claim 1.
